(19) United States
(12) Patent Application Publication
(10) Pub. No.: US 2004/0126788 A1
(43) Pub. Date: Jul. 1, 2004

(54) Fibulin-5 and uses thereof

(76) Inventors: William P. Schiemann, Denver, CO (US); Harvey F. Lodish, Brookline, MA (US)

Correspondence Address: SHERIDAN ROSS PC, 1560 BROADWAY, SUITE 1200, DENVER, CO, 80202

(21) Appl. No.: 10/640,863

(22) Filed: Aug. 13, 2003

Related U.S. Application Data
(60) Provisional application No. 60/403,292, filed on Aug. 13, 2002.

(51) Int. Cl. C12Q 1/68; G01N 33/574

(52) U.S. Cl. 435/6; 435/7.23

(57) ABSTRACT

Disclosed are methods for the use of Fibulin-5 as a marker for cancer diagnostics and other cancer screening assays, including to monitor the treatment of a patient with cancer, and for the use of Fibulin-5 as a cancer therapeutic and/or anti-angiogenesis therapeutic. Also disclosed are methods for identifying regulators of TGFβ activity and methods for identifying regulators of tumorigenicity and angiogenesis.

### CROSS-REFERENCE TO RELATED APPLICATIONS

This application claims priority under 35 U.S.C. § 119(e) from U.S. Provisional Application Serial No. 60/403,292, filed Aug. 13, 2002, entitled, “Fibulin-5 and Uses Thereof in Cancer”. The entire disclosure of U.S. Provisional Application Serial No. 60/403,292 is incorporated herein by reference.

### GOVERNMENT SUPPORT

This invention was supported in part with funding provided by NIH Grant No. CA63260 and CA095519-01A1, each awarded by the National Institutes of Health. The government may have certain rights to this invention.

### FIELD OF THE INVENTION

The present invention generally relates to the use of Fibulin-5 as a marker for cancer diagnostics and other cancer screening assays, including to monitor the treatment of a patient with cancer, and to the use of Fibulin-5 as a cancer therapeutic and/or anti-angiogenesis therapeutic. The present invention also relates to methods for identifying regulators of TGFβ activity and to methods for identifying regulators of tumorigenicity and angiogenesis.

### BACKGROUND OF THE INVENTION

The Fibulin gene family comprises 5 distinct genes that encode more than 8 protein products via alternative splicing. Fibulins are widely expressed secretory proteins found in the blood, and in the basement membranes and stroma of most tissues, where they self-associate (e.g., Fibulins 1 and 2; (1,2)) and/or interact with a variety of extracellular matrix components, including fibronectin, laminin, nidogen, aggrecan, versican, endostatin, and elastin (3-5). Thus, Fibulins likely participate in the assembly and stabilization of extracellular matrix structures; they also have been implicated in regulating organogenesis, vasculogenesis, fibrogenesis, and tumorigenesis (6-8). Although the molecular mechanisms underlying the various biological activities of Fibulins remain to be elucidated, recent work suggests that Fibulins may interact directly with cell-surface receptors, raising the possibility that these secretory proteins also function in mediating cell-cell and cell-matrix communication.

The newest member of the Fibulin family is Fibulin-5 (also referred to herein as FBLN-5; and also known as EVEC (9) or DANCE (10)), a 448 aa glycoprotein with interesting structural features: it contains an integrin-binding RGD motif, six calcium-binding EGF-like repeats, a Pro-rich insert in the first calcium-binding EGF-like repeat, and a globular C-terminal domain (9,10). Functionally, FBLN-5 binds αvβ3, αvβ5, and αvβ9 integrins (4) and mediates endothelial cell adhesion via its RGD motif (10). In response to mechanical injury, FBLN-5 expression is induced dramatically in vascular endothelial and smooth muscle cells (9,10), suggesting that FBLN-5 regulates vasculogenesis and endothelial cell function. Inactivation of the FBLN-5 gene in mice produces profound elastinopathy in the skin, lung, and vasculature (4,5), demonstrating its importance in scaffolding cells to elastic fibers.

Despite these recent advances, many questions regarding the role of FBLN-5 in mammalian biology remain to be answered, particularly (i) what are the signaling systems/molecules that regulate FBLN-5 expression; (ii) what are the effects of FBLN-5 on cell proliferation, migration, and invasion; and (iii) what are the signaling systems/molecules stimulated by FBLN-5.

### SUMMARY OF THE INVENTION

One embodiment of the invention relates to a method for assessing the tumorigenicity of cells in a patient. The method includes the steps of: (a) detecting a level of expression or activity of Fibulin-5 in a test sample from a patient to be diagnosed; and (b) comparing the level of expression or activity of Fibulin-5 in the test sample to a baseline level of Fibulin-5 expression or activity established from a control sample. Detection of a statistically significant difference in Fibulin-5 expression or activity in the test sample, as compared to the baseline level of Fibulin-5 expression or biological activity, is an indicator of a difference in the tumorigenicity or potential therefore of cells in the test sample as compared to cells in the control sample.

In one aspect of this embodiment, the step of detecting comprises detecting Fibulin-5 mRNA transcription by cells in the test sample. Such a step of detecting can be performed by a method including, but not limited to, polymerase chain reaction (PCR), reverse transcriptase-PCR (RT-PCR), in situ hybridization, Northern blot, sequence analysis, gene microarray analysis, and detection of a reporter gene. In another aspect, the step of detecting comprises detecting Fibulin-5 protein in the test sample. Such a step of detecting can be performed by a method including, but not limited to, immunoblot, enzyme-linked immunosorbant assay (ELISA), radioimmunoassay (RIA), immunoprecipitation, immunohistochemistry and immunofluorescence. In yet another aspect of this embodiment, the step of detecting comprises detecting Fibulin-5 biological activity in the test sample. Such a step of detecting can be performed by a method including, but not limited to, measuring proliferation of cells expressing Fibulin-5, detecting DNA synthesis in cells expressing Fibulin-5, detecting MAP kinase activity in cells expressing Fibulin-5, detecting MAP kinase activity in the presence of the test sample, and measuring migration and invasion ability of fibroblasts expressing Fibulin-5.

The test sample can be collected from any suitable source in the patient, including, but not limited to, a source chosen from: breast, kidney, ovary, colon, and uterus, in the patient. In one aspect, the test sample is a fibroblast cell sample.

In one aspect of this embodiment, detection of a statistically significant difference in the level of Fibulin-5 expression or activity in the test sample as compared to the baseline level, with a confidence of p<0.05, indicates that the cells in the test sample have a difference in tumorigenicity or potential therefore as compared to the control sample. In another aspect of this embodiment, detection of an at least about 10% difference in the level of Fibulin-5 expression or activity in the test sample as compared to the baseline level, with a confidence of p<0.05, indicates that the cells in the test sample have a difference in tumorigenicity or potential therefore as compared to the control sample. In another aspect, detection of an at least about 30% difference, and in another aspect, detection of at least about a 50% difference, in the level of Fibulin-5 expression or activity in the test sample as compared to the baseline level, with a confidence of p<0.05, indicates that the cells in the test sample have a difference in tumorigenicity or potential therefore as compared to the control sample. In one aspect, detection of an at least about 1.5 fold difference in the level of Fibulin-5 expression or activity in the test sample as compared to the baseline level, with a confidence of p<0.05, indicates that the cells in the test sample have a difference in tumorigenicity or potential therefore as compared to the control sample.

In one aspect of this embodiment of the invention, the test sample is from a patient being diagnosed for cancer and the baseline level is established from a control sample that is established as non-tumorigenic. In this aspect, when the Fibulin-5 expression or biological activity detected in step (b) is statistically significantly different as compared to the baseline level, the method further comprises: (c) comparing the Fibulin-5 expression or activity of the test sample as detected in step (b) to levels of Fibulin-5 expression or activity from a panel of tumor-positive control samples, wherein each of the tumor-positive control samples is correlated with a different stage of tumor development; and, (d) identifying a level of Fibulin-5 expression or activity from one of the tumor-positive control samples which is statistically significantly most similar to the level of Fibulin-5 expression or biological activity detected in step, to diagnose a stage of tumor development in the patient. In one aspect, the test sample is not a fibroblast cell sample, and a decrease in the level of Fibulin-5 expression or activity of the test sample as compared to the baseline level of expression or activity indicates that cells in the test sample are predicted to be tumorigenic or predisposed to becoming tumorigenic.

In another aspect of this embodiment of the invention, the test sample is from a patient who is known to have cancer, and the baseline level comprises a first level of Fibulin-5 expression or activity from a previous tumor cell sample from the patient and a second level of Fibulin-5 expression or activity established from a cell sample that is non-tumorigenic. In this aspect, a statistically significant change in the level of Fibulin-5 expression or activity in the test sample toward the baseline level established from the non-tumorigenic cell sample, as compared to the baseline level of expression or activity from the previous tumor cell sample, indicates that the test sample is less tumorigenic than the previous tumor cell sample; and a statistically significant change in the level of Fibulin-5 expression or activity in the test sample away from the level established from the non-tumorigenic cell sample, as compared to the baseline level of expression or activity, indicates that the test sample is more tumorigenic than the previous tumor cell sample. In this aspect, the method can further include a step (c) of modifying cancer treatment for the patient based on whether an increase or decrease in tumorigenicity is indicated in step (b).

In another aspect of this embodiment of the invention, the baseline level is established by a method selected from the group consisting of: (1) establishing a baseline level of Fibulin-5 expression or activity in an autologous control sample from the patient, wherein the autologous sample is from a same cell type, tissue type or bodily fluid type as the test sample of step (a); (2) establishing a baseline level of Fibulin-5 expression or activity from at least one previous detection of Fibulin-5 expression or activity in a previous test sample from the patient, wherein the previous test sample was of a same cell type, tissue type or bodily fluid type as the test sample of step (a); and, (3) establishing a baseline level of Fibulin-5 expression or activity from an average of control samples of a same cell type, tissue type or bodily fluid type as the test sample of step (a), the control samples having been obtained from a population of matched individuals.

Yet another embodiment of the present invention relates to an assay kit for assessing the tumorigenicity of cells in a patient, comprising: (a) a means for detecting Fibulin-5 expression or activity in a test sample; and (b) a means for detecting a control marker characteristic of a cell or tissue type that is in the test sample or that is secreted into the test sample by the cell or tissue. In one aspect, the means of (a) can include, but is not limited to, a hybridization probe of at least about 8 nucleotides that hybridizes under stringent hybridization conditions to a nucleic acid molecule encoding Fibulin-5 or a fragment thereof; an oligonucleotide primer for amplification of mRNA encoding Fibulin-5 or a fragment thereof; and an antibody that selectively binds to Fibulin-5. In one aspect, the means of (b) can include, but is not limited to, a hybridization probe of at least about 8 nucleotides that hybridizes under stringent hybridization conditions to a nucleic acid molecule encoding the control marker or a fragment thereof; an oligonucleotide primer for amplification of mRNA encoding the control marker or a fragment thereof; and an antibody that selectively binds to the control marker. In one aspect, the means of (a) and (b) are suitable for use in a method of detection selected from the group consisting of immunohistochemistry and immunofluorescence.

Yet another embodiment of the present invention relates to a method to identify a compound useful for inhibition of tumor growth or malignancy. The method includes the steps of: (a) detecting an initial level of Fibulin-5 expression or activity in a tumor cell or soluble product derived therefrom; (b) contacting the tumor cell with a test compound; (c) detecting a level of Fibulin-5 expression or activity in the tumor cell or soluble product derived therefrom after contact of the tumor cell with the compound; and, (d) selecting a compound that changes the level of Fibulin-5 expression or activity in the tumor cell or soluble product therefrom, as compared to the initial level of Fibulin-5 expression or activity, toward a baseline level of Fibulin-5 expression or activity established from a non-tumor cell, wherein the selected compound is predicted to be useful for inhibition of tumor growth or malignancy.

Another embodiment of the present invention relates to a method to reduce angiogenesis in a tissue of a patient, comprising increasing the expression or biological activity of Fibulin-5 in the cells of the tissue. In one aspect, this method can include administering Fibulin-5 or a biologically active homologue or analog thereof to the patient. In another aspect, this method can include expressing a recombinant nucleic acid molecule encoding Fibulin-5 or a homologue thereof in the tissue of the patient.

Yet another embodiment of the invention relates to a method to reduce tumorigenicity in a patient, comprising increasing the expression or biological activity of Fibulin-5 in targeted tumor cells of the patient. In one aspect, the tumor cells are from a tissue selected from the group consisting of: breast, ovary, kidney, colon, and uterus. In one aspect, the method can include administering Fibulin-5 or a biologically active homologue or analog thereof to the patient. In another aspect, the method can include expressing a recombinant nucleic acid molecule encoding Fibulin-5 or a homologue thereof in the tissue of the patient.

Another embodiment of the invention relates to a method to reduce tumorigenicity of a fibrosarcoma in a patient, comprising decreasing the expression or biological activity of Fibulin-5 in fibrosarcoma cells of the patient.

Yet another embodiment of the invention relates to a method to identify a regulator of transforming growth factor β (TGFβ). The method includes the steps of: (a) contacting a cell that expresses TGFβ and Fibulin-5 with a putative regulatory compound; (b) detecting the expression of Fibulin-5 in the cell; (c) comparing the expression of Fibulin-5 after contact with the compound to the expression of Fibulin-5 before contact with the compound, wherein detection of a change in the expression of Fibulin-5 in the cells after contact with the compound as compared to before contact with the compound indicates that the compound is a putative regulator of TGFβ.

### BRIEF DESCRIPTION OF THE DRAWINGS OF THE INVENTION

FIG. 1 shows that TGF-β-mediated induction of FBLN-5 expression was Smad2/3-dependent.

FIG. 2A shows that FBLN-5 enhances DNA synthesis in 3T3-L1 cells.

FIG. 2B shows that FBLN-5 enhances p38 MAPK protein kinase activation in 3T3 -L1 cells.

FIG. 2C shows that FBLN-5 enhances ERK1/2 protein kinase activation in 3T3-L1 cells.

FIG. 3A shows that FBLN-5 expression in combination with TGF-β treatment significantly increased DNA synthesis in HT1080 cells.

FIG. 3B shows that FBLN-5 expression significantly increased the migration of HT1080 cells toward fibronectin.

FIG. 3C shows the invasion of GFP- or FBLN-5-expressing HT1080 cells through Matrigel-coated membranes.

FIG. 4A shows that FBLN-5 expression significantly decreased DNA synthesis in Mv1Lu cells.

FIG. 4B shows that FBLN-5 was able to significantly reduce cyclin A-luciferase expression in unstimulated Mv1Lu cells.

FIG. 5A shows that FBLN-5 in combination with TGF-β treatment synergistically stimulated AP-1 activity.

FIG. 5B shows that stimulation of AP-1 activity by FBLN-5 and TGF-β was readily inhibited by overexpression in Mv1Lu cells of dominant-negative versions of either MKK1 or p38 MAPKα.

FIG. 5C shows that starved FBLN-5-expressing Mv1Lu cells exhibited significantly higher activities of ERK1/2 than control cells.

FIG. 5D shows that starved FBLN-5-expressing Mv1Lu cells exhibited significantly higher activities of p38 MAPK than control cells.

FIG. 6 shows that Fibulin-5 expression is downregulated during MB114 endothelial cell tubule morphogenesis.

FIG. 7 shows that TGF-β stimulates Fibulin-5 expression in MB114 endothelial cells.

FIG. 8 shows that Fibulin-5 inhibits endothelial cell DNA synthesis.

FIG. 9A shows that Fibulin-5 inhibits endothelial cell migration.

FIG. 9B shows that Fibulin-5 inhibits endothelial cell invasion.

FIG. 10 shows that Fibulin-5 stimulates the expression of the angiostatic protein thrombospondin (TSP-1).

FIG. 11 shows that Fibulin-5 inhibits endothelial cell DNA synthesis stimulated by VEGF.

FIG. 12 shows that Fibulin-5 inhibits p38 MAPK activation stimulated by VEGF in MB114 endothelial cells.

FIG. 13 shows that Fibulin-5 inhibits ERK1/2 activation stimulated by VEGF in MB114 endothelial cells.

### DETAILED DESCRIPTION OF THE INVENTION

The present invention generally relates to the use of Fibulin-5 as a marker for cancer diagnostics and other cancer screening assays, including to monitor the treatment of a patient with cancer, and to the use of Fibulin-5 as a cancer therapeutic and/or anti-angiogenesis therapeutic. The present inventor has discovered that Fibulin-5 is a TGFβ-secretory protein and has aberrant expression in various tumor cells (primarily downregulated). Therefore, a change in Fibulin-5 expression or activity in a cell as compared to a normal control for the cell is a useful marker for cancer diagnostic assays and cancer screening assays. In addition, the present invention is directed to the up- or down-regulation, restoration, or replacement of Fibulin-5 in cells of a patient by protein administration, drug administration or gene therapy as a method of treating cancer and as a method of inhibiting angiogenesis.

The biology of TGF-β can largely be divided into two broad categories: regulation of cell cycling versus regulation of cell microenvironments. Although the ability of TGF-β to inhibit cell cycling and therefore to suppress tumor formation has been thoroughly established (20,21), less is known concerning its role in regulating cell microenvironments. Indeed, while TGF-β is clearly an important player governing the production of cytokines, growth factors, and extracellular matrix proteins by a variety of cell types and tissues, the identities these proteins and their role in mammalian biology remain to be fully elucidated. The present inventors therefore sought to identify these secretory proteins, believing that in doing so and in determining their function the understanding of the molecular mechanisms that underlie both the biology and pathology of these secretory proteins would be significantly strengthened, and perhaps that of TGF-β as well.

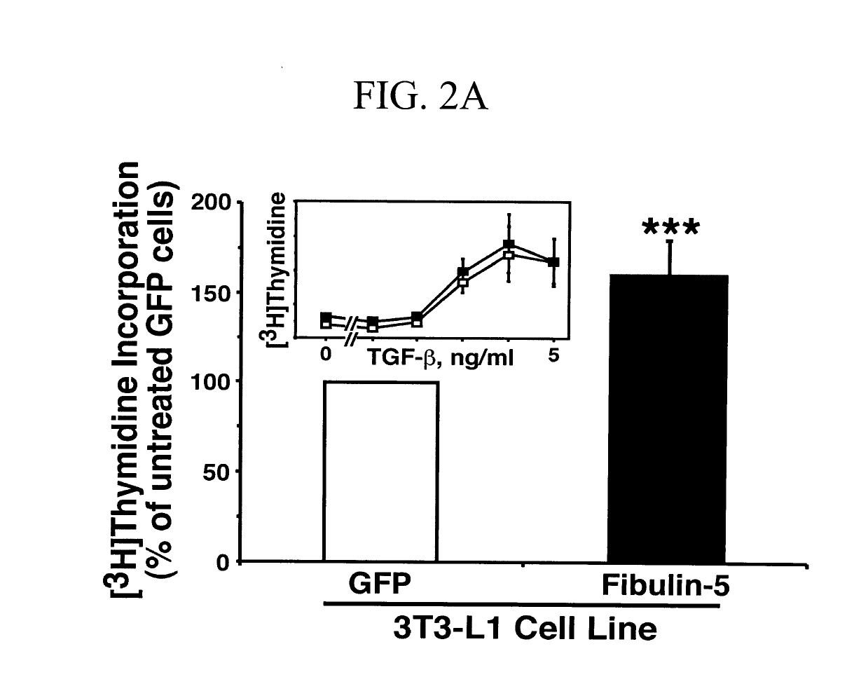

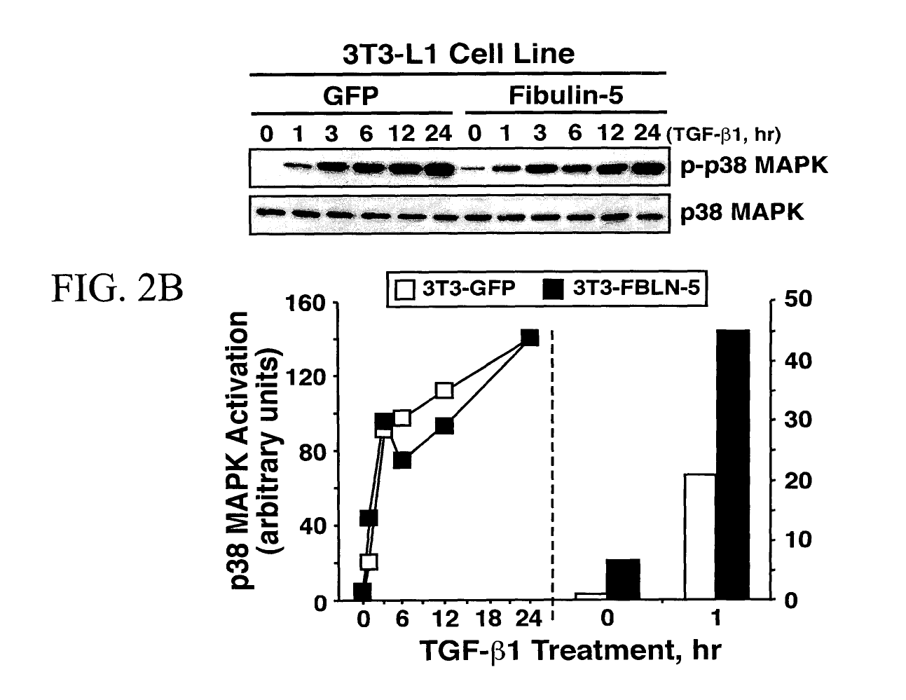

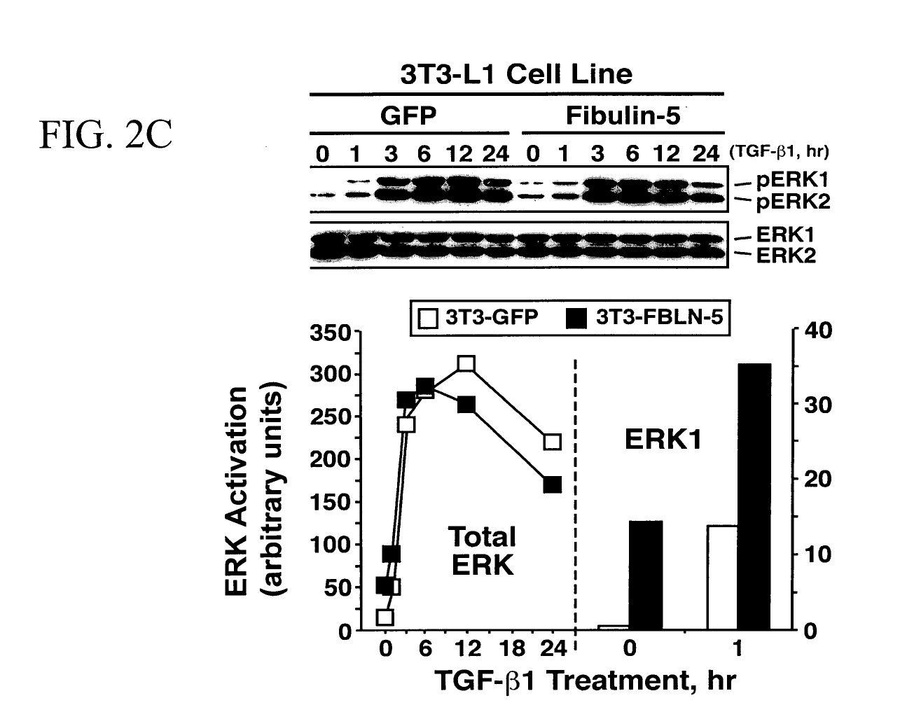

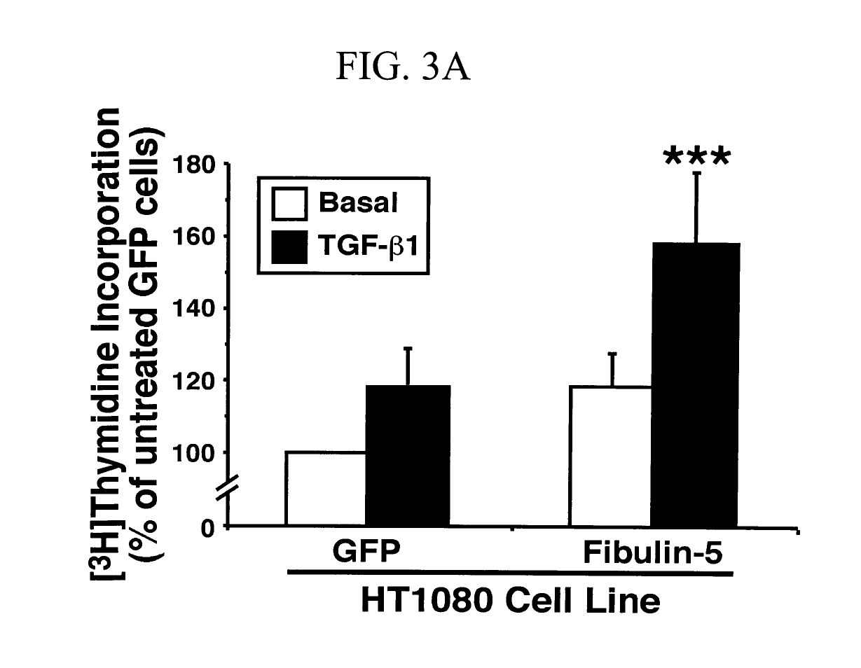

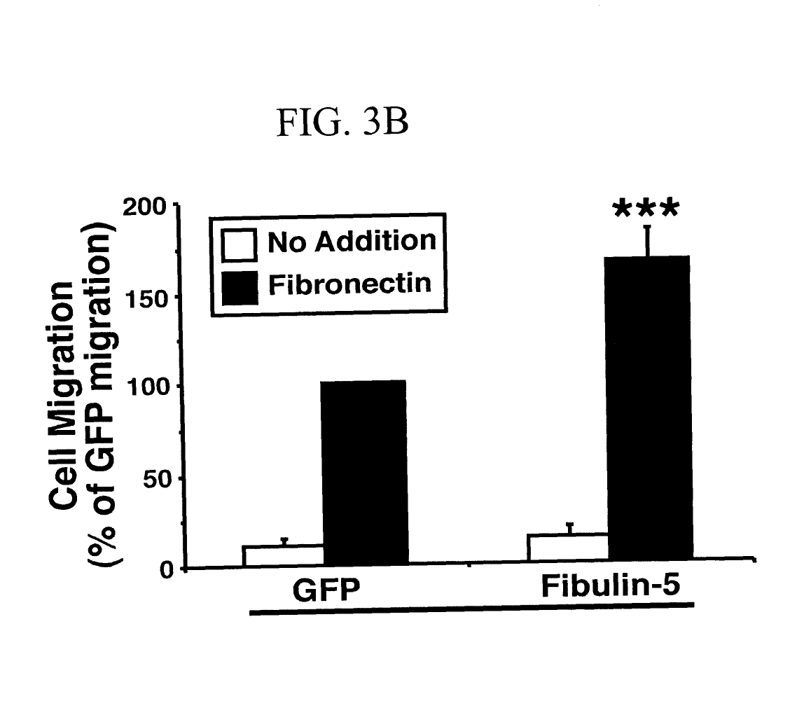

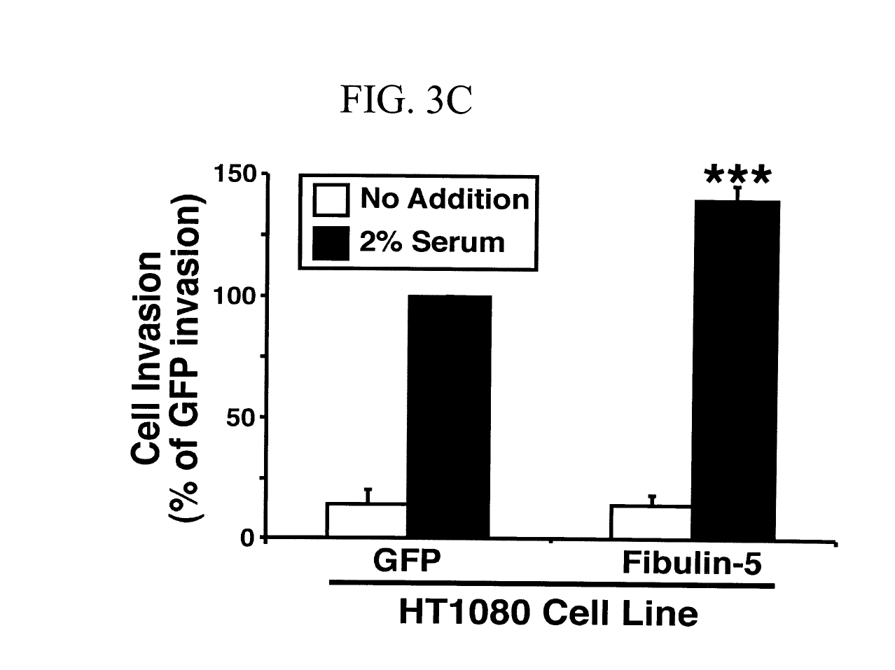

To this end, the present inventors now present FBLN-5 as a novel gene target for TGF-β in fibroblasts and endothelial cells. Moreover, our findings have also identified the TGF-β receptor system as being the first signaling system coupled to FBLN-5 expression. The inventors also show for the first time that FBLN-5 expression (i) regulates proliferation in a context-specific manner (FIGS. 2, 3 and 4); (ii) enhances the growth, motility, and invasion of human fibrosarcoma cells (FIG. 3); (iii) is aberrant in the majority of metastatic human malignancies, and (iv) stimulates MAP kinases that enhance AP-1 activity stimulated by TGF-β (FIGS. 2, 4 and 5). Whereas previous studies implicated FBLN-5 in mediating the assembly and stabilization of extracellular matrix structures (4,5), the present inventors' findings demonstrate that FBLN-5 also functions as a multifunctional signaling molecule capable of propagating messages between cells or between matrix and cells.

The biological significance of Fibulin family members and the molecular mechanisms whereby they mediate these activities have yet to be fully appreciated. Based on their expression patterns in fetal and adult tissues, Fibulins have been suggested to regulate a variety of normal and abnormal biological processes, including organogenesis, thrombosis, fibrogenesis, and tumorigenesis (6-8). Gene targeting experiments in mice support this assertion. For instance, Fibulin-1-deficient mice suffer perinatal lethality due to impaired endothelial function, resulting in fatal hemorrhaging in neural and epidermis tissues; they also exhibit malformations of the kidney and lung (3). Thus, Fibulin-1 is essential for proper development of the kidneys, lungs, and vascular system. Interestingly, FBLN-5-null mice also exhibit lung and vasculature malformations (4,5); however, these developmental deficiencies are distinct from those resulting from Fibulin-1 inactivation, suggesting that expression of Fibulins 1 and 5 fulfill similar, yet non-overlapping functions during development. FBLN-5-deficient mice are also distinguished from their Fibulin-1-null counterparts by their profound elastinopathy of the skin, which is reminiscent of cutis laxa syndrome in humans (4,5).

Additional insights into the function of FBLN-5 are suggested by its expression patterns. For instance, the inventors (see Example 1) and others (9,10) find that FBLN-5 is widely expressed throughout human and murine tissues. FBLN-5 expression is also induced significantly during embryogenesis (see Example 1), predominantly in developing arteries and mesenchymal tissues, and in migrating neural crest cells (9,10). These findings suggest that FBLN-5 may be an important regulator of epithelial-to-mesenchymal transdifferentiation. Consistent with this idea, FBLN-5, like other Fibulins (6,29-31), localizes to boundaries between epithelium and mesenchyme (9,10). Thus, FBLN-5 likely plays a role during tissue development, remodeling, and repair. Accordingly, FBLN-5 expression is induced dramatically in vascular endothelial and smooth muscle cells in response to mechanical injury (9,10), and in uterine myometrial arteries undergoing cyclic angiogenesis (9). Moreover, the present inventors have recently found that retroviral delivery of FBLN-5 to animal wounds significantly increased granulation tissue volume, thereby enhancing wound closure and healing. This observation is reminiscent of the effects of wounding on Fibulin-2 expression, which is significantly upregulated throughout the granulation layer (33). Although the mechanism by which FBLN-5 promotes wound healing has not yet been fully elucidated, the inventors' findings demonstrating that FBLN-5 regulates proliferation, migration, and invasion are entirely consistent with its designation as a positive mediator of tissue remodeling and wound healing. Future studies clearly need to address which cell types are targeted by FBLN-5 during wound healing and, more importantly, what signaling systems are activated in them by FBLN-5. Because TGF-β stimulates wound healing and FBLN-5 expression, it will also be interesting to determine the role of FBLN-5 in the context of TGF-β-mediated tissue remodeling and wound healing.

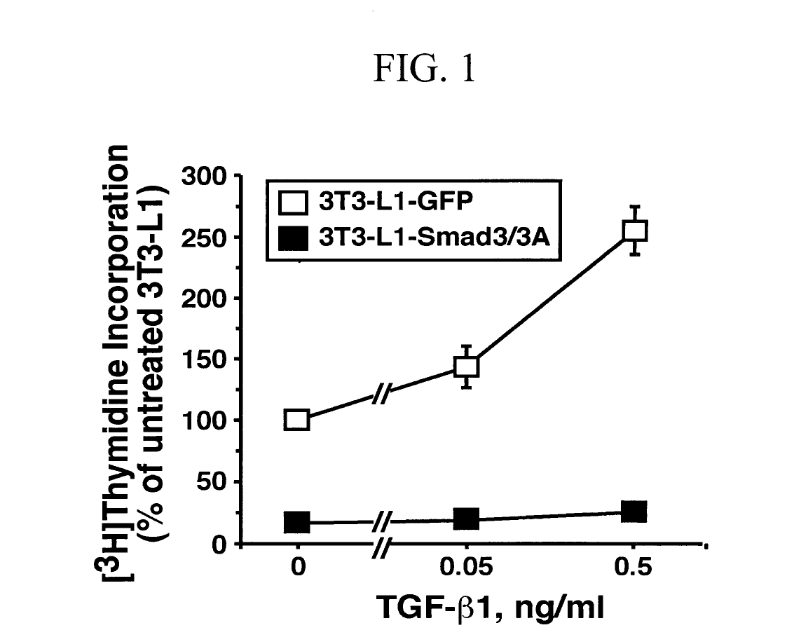

Endothelial cells are clearly targets of FBLN-5 (see Examples). For instance, endothelial cells express and secrete FBLN-5, especially in response to mechanical injury (9,10). Moreover, FBLN-5 mediates endothelial cell adhesion by binding αvβ3, αvβ5, and αvβ9 integrins via its RGD motif (4,10). These findings indicate that FBLN-5 functions in an autocrine manner to regulate endothelial cell activities and vasculogenesis. TGF-β is a potent regulator of endothelial activities [e.g., proliferation, migration, invasion, and tubule formation; (34)] and, as shown herein, a stimulator of FBLN-5 expression in endothelial cells (FIG. 1). As such, without being bound by theory, the present inventors suggest that FBLN-5 expression participates in mediating some of the effects of TGF-β on endothelial cells. It therefore will be interesting to determine precisely how FBLN-5 signaling affects endothelial cell activities, and ultimately to examine the relative contribution of these events during endothelial cell activation by TGFβ.

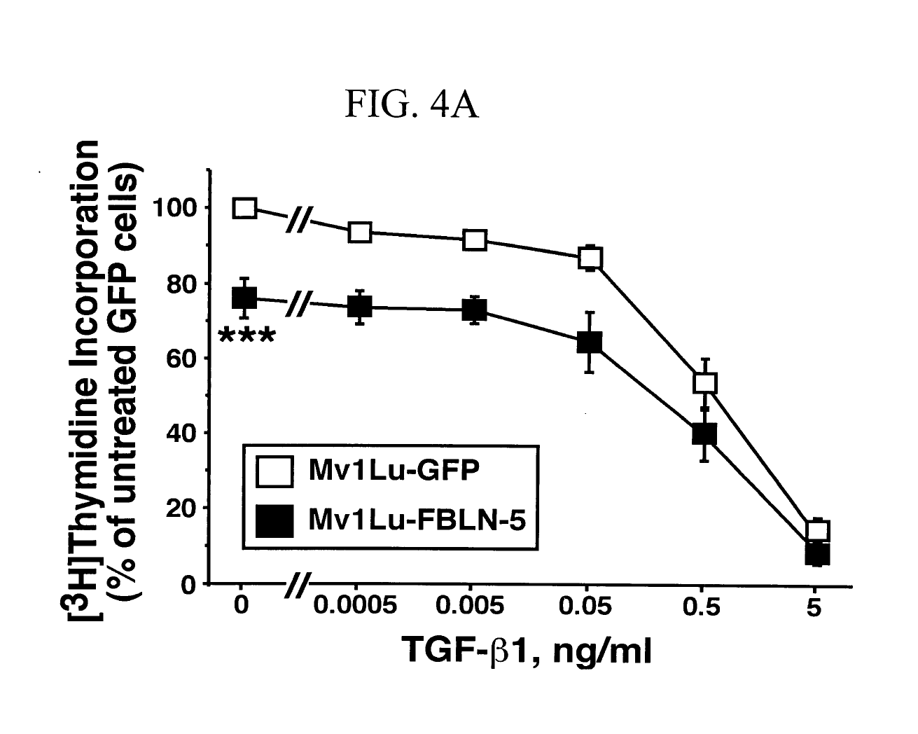

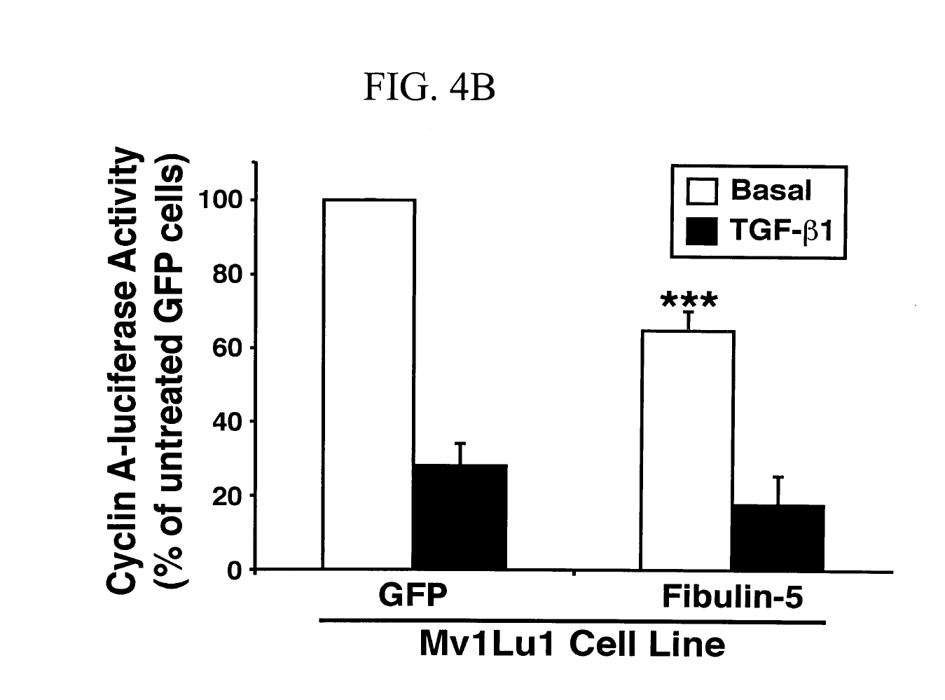

The finding that Fibulin family members, including FBLN-5, are highly expressed at the boundaries between epithelial and mesenchymal cells (9,10,29-31) prompted the inventors to investigate whether FBLN-5 expression serves to mediate one set of biological activities in fibroblasts, while mediating a distinctly different set of activities in neighboring epithelial cells. The inventors have now shown that FBLN-5 expression regulates proliferation in a cell type-specific manner, stimulating DNA synthesis in fibroblasts while inhibiting that in epithelial cells (FIGS. 2 and 4). Interestingly, in both cell types FBLN-5 expression led to activation of MAP kinases (i.e., ERK1/2 and p38 MAPK), whose activities figure prominently in a variety of physiological processes including the stimulation or inhibition of cell proliferation (25,26). With respect to epithelial cells, the inventors found that FBLN-5 expression synergized with TGF-β in stimulating AP-1 activity, a response that required the activities of ERK1/2 and p38 MAPK. Moreover, FBLN-5 also repressed cyclin A expression in Mv1Lu cells, thereby contributing to their reduced synthesis of DNA. Without being bound by theory, the present inventors anticipate that FBLN-5 expression and its consequential stimulation of ERK1/2 and p38 MAPK activities will also induce AP-1 activity in 3T3-L1 cells. Along these lines, preliminary experiments by the inventors have shown that FBLN-5 expression elevates cyclin A expression in 3T3-L1 cells (data not shown), a finding consistent with the ability of FBLN-5 to promote DNA synthesis in these cells. Recently, Bhowmick et al (35) determined that TGF-β-mediated epithelial-to-mesenchymal transdifferentiation and p38 MAPK activation required β1 integrin expression. Although binding of FBLN-5 to β1 integrins has yet to be established, it is nonetheless possible that engagement of αvβ3, αvβ5, or αvβ9 integrins by FBLN-5 similarly serves in mediating MAP kinase activation by TGF-β. Thus based on the present inventors' findings, it is proposed that engagement of integrins by FBLN-5 results in the activation of a conserved signaling system(s) whose ultimate biological outcome depends upon the genetic makeup of the individual cell in question. In other words, FBLN-5-mediated stimulation of MAP kinases leads to the activation of distinct forms of AP-1 dimers that arise from cell type-specific expression of Jun, Fos, and ATF members, which ultimately results in context-specific effects on gene expression and cellular activities.

As a Fibulin family member, FBLN-5 is not unique in its ability to regulate proliferation in a context-specific manner. For instance, Fibulin-3 expression is elevated in fibroblasts undergoing growth arrest or senescence (23), indicating its involvement in inhibiting cell cycle progression. However, microinjection of Fibulin-3 mRNA into fibroblasts stimulates DNA synthesis in the injected cells and in their non-injected neighbors. Thus, Fibulin-3 regulates cell growth in a context-specific manner via autocrine and paracrine signaling mechanisms. Likewise, Fibulin-4 expression promotes the growth of normal and abnormal cells through p53-independent and -dependent mechanisms, respectively. With respect to the latter, signal sequence polymorphisms in the Fibulin-4 gene prevent its secretion from human colon cancer cells (28). This intracellular variant of Fibulin-4 then interacts with and inhibits the activity of p53, leading to enhanced proliferation of tumor cells (22). Thus, expression of Fibulins 3 and 4, like that of FBLN-5, governs proliferation in a cell type-specific manner.

The present inventors' findings have identified FBLN-5 as a regulator of tumorigenesis. The inventors have shown that FBLN-5 enhances the malignancy of human HT1080 fibrosarcoma cells by (i) increasing their DNA synthesis in response to TGF-β; (ii) enhancing their migration towards fibronectin (FIG. 3B) and laminin (data not shown); and (iii) augmenting their invasion through synthetic basement membranes (FIG. 3B). Accordingly, tumors of the majority of patients surveyed expressed FBLN-5 aberrantly (44/68 cases); however, contrary to the expectations based on the results with fibrosarcomas, FBLN-5 expression was downregulated in 95% of these cases, particularly in cancers of the kidney, breast, ovary, colon and uterus (see Example 4). More importantly, FBLN-5 expression was inversely related to tumor metastasis, being expressed aberrantly in ˜65% (17/25 cases) of metastatic malignancies and being downregulated in 100% of these cases (Example 4). Taken together, the present inventors' findings highlight the context-specific nature of FBLN-5 in regulating the activities of normal (i.e., non-cancerous) and abnormal (i.e., cancerous) cells. Moreover, the striking downregulation of FBLN-5 expression in human tumors suggests that FBLN-5 functions predominantly to suppress, not promote, tumor formation. It is interesting to note that TGF-β both suppresses and promotes tumor formation in a context-specific manner (20,36).

Furthermore, given the discoveries of the present invention, one can now ascertain the identity of the cells whose expression of FBLN-5 becomes aberrant during tumorigenesis. Whether downregulation of FBLN-5 in these cells results from altered TGF-β signaling or additional compensatory mechanisms (i.e., elevated FBLN-5 protein expression diminishes FBLN-5 mRNA expression via a negative feedback loop) is currently unknown. Regardless, the studies presented herein demonstrate that alterations in tumor microenvironments negatively impacts FBLN-5 expression, an event capable of stimulating (e.g., fibrosarcoma cells) or inhibiting (e.g., cancers of the kidney, breast, ovary, and colon) tumorigenesis in a context-specific manner. A similar paradox has been described for Fibulin-3, whose expression positively and negatively regulates cell growth, and is upregulated in transformed cell lines (23). Likewise, Fibulin-1 expression has been shown to inhibit the tumorigenicity of fibrosarcoma cells (18,24). However in ovarian cancer, Fibulin-1 expression is elevated significantly, leading to the suggestion that Fibulin-1 expression may enhance ovarian cancer metastasis (37), and serve as a prognostic indicator for disease risk and aggressiveness (38). Thus, expression of Fibulins 1, 3, and 4 (see above), like that of FBLN-5, regulates tumorigenesis in a context- and cell type-specific manner.

Finally, as described in Example 7, the present inventors have shown the Fibulin-5 inhibits angiogenic sprouting in endothelial cells as well as DNA synthesis and additionally inhibits endothelial cell migration and invasion. The inventors have demonstrated the Fibulin-5 stimulates the expression of the angiostatic protein, thrombospondin (TSP-1), and inhibits endothelial cell DNA synthesis, p38 MAPK activation, and ERK1/2 activation that are stimulated by VEGF in such cells.

In summary, the present inventors have established FBLN-5 as a novel gene target for TGF-β in fibroblasts and endothelial cells. FBLN-5 expression regulated proliferation in a cell type-specific manner, in part through its ability to stimulate MAP kinases and AP-1 activity. While enhancing the tumorigenicity of human fibrosarcoma cells, FBLN-5 expression was downregulated dramatically during carcinogenesis, pointing towards a prominent role in mediating tumor suppression. In addition, a role for FBLN-5 in the inhibition of angiogenesis has also been demonstrated. Indeed, in terms of disease development, without being bound by theory, the present inventors expect that the inappropriate absence or presence of FBLN-5 in cell microenvironments will elicit profound effects on a variety of cellular activities and processes, particularly those involved in tissue development, remodeling, and repair.

One embodiment of the present invention relates to a method (i.e., an assay) for assessing tumorigenicity of cells in a patient. Such a method includes the steps of: (a) detecting a level of expression or activity of Fibulin-5 in a test sample from a patient to be diagnosed; and (b) comparing the level of expression or activity of Fibulin-5 in the test sample to a baseline level of Fibulin-5 expression or activity established from a control sample. Detection of a statistically significant difference in Fibulin-5 expression or activity in the test sample, as compared to the baseline level of Fibulin-5 expression or biological activity, is an indicator of a difference in the tumorigenicity or potential therefore of cells in the test sample as compared to cells in the control sample. As discussed above, expression of Fibulin-5 is cell- and context-specific. Therefore, Fibulin-5 expression or activity could be either upregulated or downregulated in a cell as compared to the control. In most tumor cell types, Fibulin-5 will be downregulated as compared to a normal (non-tumor) cell of the same cell type. Therefore, in one aspect of the invention, detection of reduced Fibulin-5 expression or reduced Fibulin-5 biological activity as compared to the baseline level of Fibulin-5 expression or biological activity, is an indicator of increased tumorigenicity or potential therefore of cells in the test sample. In this aspect of the invention, the cells in the test sample might be from, for example, breast, kidney, colon, ovary, uterus, or a metastatic cancer. In another aspect of the invention, detection of increased Fibulin-5 expression or increased Fibulin-5 biological activity as compared to the baseline level of Fibulin-5 expression or biological activity, is an indicator of decreased tumorigenicity or potential therefore by the cells in the test sample. In this latter embodiment, for example, the cells in the test sample might be fibroblasts. In either case, detection of substantially the same Fibulin-5 expression or biological activity (i.e., differences between sample and baseline control are not statistically significant with a degree of confidence of p<0.05) indicates no significant change or difference in tumorigenicity or the potential therefore by the cell in the test sample (i.e., relative to the baseline control). The method of the present invention can be used for any type of tumor wherein Fibulin-5 activity is found to be statistically significantly changed in tumor cells as compared to the corresponding normal cells.

According to the present invention, the phrase “tumorigenicity” refers primarily to the tumor status of a cell (i.e., the extent of neoplastic transformation of a cell, the malignancy of a cell, or the propensity for a cell to form a tumor and/or have characteristics of a tumor), which is a change of a cell or population of cells from a normal to malignant state. The change typically involves cellular proliferation at a rate which is more rapid than the growth observed for normal cells under the same conditions, and which is typically characterized by one or more of the following traits: continued growth even after the instigating factor (e.g., carcinogen, virus) is no longer present; a lack of structural organization and/or coordination with normal tissue, and typically, a formation of a mass of tissue, or tumor. A tumor, therefore, is most generally described as a proliferation of cells (e.g., a neoplasia, a growth, a polyp) resulting from neoplastic growth and is most typically a malignant tumor. In the case of a neoplastic transformation, a neoplasia is malignant or is predisposed to become malignant. Malignant tumors are typically characterized as being anaplastic (primitive cellular growth characterized by a lack of differentiation), invasive (moves into and destroys surrounding tissues) and/or metastatic (spreads to other parts of the body). As used herein, reference to a “potential for neoplastic transformation”, “potential for tumorigenicity” or a “potential for tumor cell growth” refers to an expectation or likelihood that, at some point in the future, a cell or population of cells will display characteristics of neoplastic transformation, including rapid cellular proliferation characterized by anaplastic, invasive and metastatic growth. In the present invention, the expectation or likelihood of tumorigenicity or neoplastic transformation and particularly malignant tumor cell growth (i.e., a positive diagnosis of tumorigenicity) is determined based on a detection of aberrant expression or activity of Fibulin-5 in a cell as compared to a baseline (i.e., control) level of Fibulin-5 expression or biological activity that is considered to be representative of Fibulin-5 expression or biological activity in a normal (not neoplastically transformed) cell, as discussed in detail below.

This method of the present invention has several different uses. First, the method can be used to diagnose tumorigenicity, or the potential for tumorigenicity, in the cells of a patient. The patient can be an individual who is suspected of having a tumor, or an individual who is presumed to be healthy, but who is undergoing a routine or diagnostic screening for tumor growth. The patient can also be an individual who has previously been diagnosed with cancer and treated, and who is now under surveillance for recurring tumor growth. The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, has been identified. In contrast, a “negative diagnosis” indicates that the disease or condition, or a potential for developing the disease or condition, has not been identified. Therefore, in the present invention, a positive diagnosis (i.e., a positive assessment) of tumor growth or tumorigenicity (i.e., malignant or inappropriate cell growth or neoplastic transformation), or the potential therefor, means that the indicators (e.g., signs, symptoms) of tumor growth according to the present invention (i.e., a change in Fibulin-5 expression or biological activity as compared to a baseline control) have been identified in the sample obtained from the patient. Such a patient can then be prescribed treatment to reduce or eliminate the tumor growth. Similarly, a negative diagnosis (i.e., a negative assessment) for tumor growth or a potential therefore means that the indicators of tumor growth or a likelihood of developing tumor growth as described herein (i.e., a change in Fibulin-5 expression or biological activity as compared to a baseline control) have not been identified in the sample obtained from the patient. In this instance, the patient is typically not prescribed any treatment, but may be reevaluated at one or more timepoints in the future to again assess tumor growth. Baseline levels for this particular embodiment of the method of assessment of tumorigenicity of the present invention are typically based on a “normal” or “healthy” sample from the same bodily source as the test sample (i.e., the same tissue, cells or bodily fluid), as discussed in detail below.

In a second embodiment, the method of the present invention can be used more specifically to “stage” a tumor in a patient. Therefore, the patient can be diagnosed as having a tumor or potential therefore by the method as discussed above, or by any other suitable method (e.g, physical exam, X-ray, CT scan, blood test for a tumor antigen, surgery), and then (or at the same time, when the present method is also used as a diagnostic), the method of the present invention can be used to determine the stage of progression of tumor growth in an individual. For most cancer types, standard staging criteria exist and are known in the art. For example, in breast tumors, there are five different general stages of tumor development which are known and acknowledged in the art as stages 0, I, II, III and IV (although these stages can be grouped into more complex subgroups based on more specific indicators). In this embodiment of the method of the present invention, the Fibulin-5 expression and/or biological activity in the patient sample is compared to a panel of several different “baseline” levels of Fibulin-5 expression or biological activity, wherein each baseline level represents a previously established level for a given stage of the cancer being diagnosed. For example, for a breast tumor staging assay, baseline levels of Fibulin-5 expression and/or biological activity can be established for Stages I, II, III and IV of breast tumor cells (e.g., using an average level determined from a random sampling of tumors from different patients at the various stages). Therefore, in this embodiment, the level of expression of Fibulin-5 expression or biological activity in the patient sample is compared to the various baseline levels corresponding to the different stages of tumor growth to identify the baseline level that is statistically closest to the level of Fibulin-5 expression or biological activity detected in the patient. The ability to “stage” a tumor in the method of the present invention allows the physician to more appropriately prescribe treatment for the patient.

In a third embodiment of this method of the present invention, the method is used to monitor the success, or lack thereof, of a treatment for cancer in a patient that has been diagnosed as having cancer. In this embodiment, the baseline level of Fibulin-5 expression or biological activity typically includes the previous level of Fibulin-5 expression or biological activity in a sample of the patient's tumor, so that a new level of Fibulin-5 expression or biological activity can be compared to determine whether tumor cell growth is decreasing, increasing, or substantially unchanged as compared to the previous, or first sample (i.e., the initial sample which presented a positive diagnosis). In addition, or alternatively, a baseline established as a “normal” or “healthy” level of Fibulin-5 expression or biological activity can be used in this embodiment, particularly to determine in what manner Fibulin-5 expression is regulated in tumors for the given cell type. This embodiment allows the physician to monitor the success, or lack of success, of a treatment that the patient is receiving for cancer, and can help the physician to determine whether the treatment should be modified. In one embodiment of the present invention, the method includes additional steps of modifying cancer treatment for the patient based on whether an increase or decrease in tumor cell growth is indicated by evaluation of Fibulin-5 expression and/or biological activity in the patient.

The first step of the method of the present invention includes detecting Fibulin-5 expression or biological activity in a test sample from a patient. According to the present invention, the term “test sample” can be used generally to refer to a sample of any type which contains cells or products that have been secreted from cells (i.e., Fibulin-5 is a secreted protein and so one can evaluate a cell supernate, bodily fluid or other media into which Fibulin-5 may have been secreted by a cell) to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells which are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for Fibulin-5 expression or biological activity and/or contains the soluble Fibulin-5 secreted by cells, and is a fluid obtained by any method suitable for the particular bodily fluid to be sampled. Bodily fluids suitable for sampling include, but are not limited to, blood, mucous, seminal fluid, saliva, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the breast, the sample can be a sample of epithelial cells from a biopsy (i.e., a cell sample) or a breast tissue sample from a biopsy (a tissue sample). The sample that is most useful in the present invention will be cells, tissues or bodily fluids isolated from a patient by a biopsy or surgery or routine laboratory fluid collection.

Once a sample is obtained from the patient, the sample is evaluated for detection of Fibulin-5 expression or biological activity in the cells of the sample. The phrase “Fibulin-5 expression” can generally refer to Fibulin-5 mRNA transcription or Fibulin-5 protein translation. Preferably, the method of detecting Fibulin-5 expression or biological activity in the patient is the same or qualitatively equivalent to the method used for detection of Fibulin-5 expression or biological activity in the sample used to establish the baseline level.

Methods suitable for detecting Fibulin-5 transcription include any suitable method for detecting and/or measuring mRNA levels from a cell or cell extract. Such methods include, but are not limited to: polymerase chain reaction (PCR), reverse transcriptase PCR (RT-PCR), in situ hybridization, Northern blot, sequence analysis, gene microarray analysis (gene chip analysis) and detection of a reporter gene. Such methods for detection of transcription levels are well known in the art, and many of such methods are described in detail below (See Northern blot analysis described in Examples), in Sambrook et al., Molecular Cloning: A Laboratory Manual, Cold Spring Harbor Labs Press, 1989 and/or in Glick et al., Molecular Biotechnology: Principles and Applications of Recombinant DNA, ASM Press, 1998; Sambrook et al., ibid., and Glick et al., ibid. are incorporated by reference herein in their entireties. The nucleotide and amino acid sequence for Fibulin-5 in different mammalian species can be found in public sequence databases, such as GenBank in the National Center for Biotechnology Information. The nucleic acid sequence for the coding region of human Fibulin-5 is represented herein by SEQ ID NO:2. SEQ ID NO:2 encodes a human Fibulin-5 protein having the amino acid sequence represented by SEQ ID NO:3. The nucleic acid sequence for the coding region of murine Fibulin-5 is represented herein by SEQ ID NO:4. SEQ ID NO:4 encodes a murine Fibulin-5 protein having the amino acid sequence represented by SEQ ID NO:5. Measurement of Fibulin-5 transcription is suitable when the sample is a cell or tissue sample; therefore, when the sample is a bodily fluid sample containing cells or cellular extracts, the cells are typically isolated from the bodily fluid to perform the expression assay, or the fluid is evaluated for the presence of secreted Fibulin-5 protein.

Fibulin-5 expression can also be identified by detection of Fibulin-5 translation (i.e., detection of Fibulin-5 protein in a sample). Methods suitable for the detection of Fibulin-5 protein include any suitable method for detecting and/or measuring proteins from a cell or cell extract. Such methods include, but are not limited to, immunoblot (e.g., Western blot), enzyme-linked immunosorbant assay (ELISA), radioimmunoassay (RIA), immunoprecipitation, immunohistochemistry and immunofluorescence. Particularly preferred methods for detection of proteins include any single-cell assay, including immunohistochemistry and immunofluorescence assays. Such methods are well known in the art. Furthermore, antibodies against Fibulin-5 are known in the art and are described in the public literature and methods for production of antibodies against Fibulin-5 are well known in the art.

The term, “Fibulin-5 biological activity” or “Fibulin-5 activity” refers to any biological action of the Fibulin-5 protein, including, but not limited to, regulation of proliferation of cells expressing Fibulin-5, regulation of DNA synthesis in cells expressing Fibulin-5, regulation of MAP kinase activity in cells expressing Fibulin-5, and regulation of migration and invasion ability of fibroblasts expressing Fibulin-5. Methods to detect Fibulin-5 biological activity are known in the art and described in detail in the Examples section and include, but are not limited to, assays for the detection of any of the above-identified activities. Procedures for the isolation and assay of several Fibulin's, including Fibulin-5 have been described in the art (e.g., Markova et al., Am J Hum Genet. April 2003;72(4):998-1004; Loeys et al., Hum Mol Genet. Sep. 1, 2002;11(18):2113-8; Kapetanopoulos et al., Mol Genet Genomics June 2002;267(4):440-6; Schiemann et al., J Biol Chem. Jul. 26, 2002;277(30):27367-77; Midwood et al., Curr Biol. Apr. 16, 2002;12(8):R279-81; Nakamura et al., Nature. Jan. 10, 2002;415(6868):171-5; Yanagisawa et al., Nature. Jan. 10, 2002;415(6868):168-71; Jean et al., J Physiol Lung Cell Mol Physiol. January 2002;282(1):L75-82; Kowal et al., Cytogenet Cell Genet. 1999;87(1-2):2-3; Nakamura et al., J Biol Chem. Aug. 6, 1999;274(32):22476-83; and Kowal et al., Circ Res. May 28, 1999;84(10):1166-76; each of which is incorporated by reference in its entirety). Other methods for detection of Fibulin-5 biological activity, will be known to those of skill in the art and are encompassed by the present invention, and methods for detection of Fibulin-5 activity are described in the Examples section.

The method of the present invention includes a step of comparing the level of Fibulin-5 expression or biological activity detected in step (a) to a baseline level of Fibulin-5 expression or biological activity established from a control sample. According to the present invention, a “baseline level” is a control level, and in some embodiments (but not all embodiments, depending on the method), a normal level, of Fibulin-5 expression or activity against which a test level of Fibulin-5 expression or biological activity (i.e., in the test sample) can be compared. Therefore, it can be determined, based on the control or baseline level of Fibulin-5 expression or biological activity, whether a sample to be evaluated for tumor cell growth has a measurable increase, decrease, or substantially no change in Fibulin-5 expression or biological activity, as compared to the baseline level. As discussed above, the baseline level can be indicative of a different states of cell tumorigenicity or lack thereof, depending on the primary use of the assay. For example, the baseline level can be indicative of the cell growth expected in a normal (i.e., healthy, negative control, non-tumor) cell sample. Therefore, the term “negative control” used in reference to a baseline level of Fibulin-5 expression or biological activity typically refers to a baseline level established in a sample from the patient or from a population of individuals which is believed to be normal (i.e., non-tumorous, not undergoing neoplastic transformation, not exhibiting inappropriate cell growth). It is noted that the “negative control” most typically has a higher level of Fibulin-5 expression or activity than would be detected in an experimental cell having inappropriate, increased cell growth, because the Fibulin-5 expression/biological activity and cell growth are inversely related in most tumor cell types. However, in some cell types (e.g., fibroblasts), the negative control may have a lower level of Fibulin-5 expression or activity than the tumor type. In another embodiment, a baseline can be indicative of a positive diagnosis of tumor cell growth. Such a baseline level, also referred to herein as a “positive control” baseline, refers to a level of Fibulin-5 expression or biological activity established in a cell sample from the patient, another patient, or a population of individuals, wherein the sample was believed, based on data for that cell sample, to be neoplastically transformed (i.e., tumorous, exhibiting inappropriate cell growth, cancerous). It is noted that this “positive control” will most typically actually have a lower level of Fibulin-5 expression or activity than in a normal cell, again due to the inverse relationship between Fibulin-5 and cell growth in the majority of tumor cells. As discussed above with regard to the negative control, the inverse can be true for some cell types, such as fibroblasts. In one aspect of this embodiment, the baseline can be indicative of a particular stage of tumor cell growth, which will allow a patient's sample to be “staged” (i.e., the stage of the cancer in the patient can be identified). In yet another embodiment, the baseline level can be established from a previous sample from the patient being tested, so that the tumor growth of a patient can be monitored over time and/or so that the efficacy of a given therapeutic protocol can be evaluated over time. Methods for detecting Fibulin-5 expression or biological activity are described in detail above.

The method for establishing a baseline level of Fibulin-5 expression or activity is selected based on the sample type, the tissue or organ from which the sample is obtained, the status of the patient to be evaluated, and, as discussed above, the focus or goal of the assay (e.g., diagnosis, staging, monitoring). Preferably, the method is the same method that will be used to evaluate the sample in the patient. In a most preferred embodiment, the baseline level is established using the same cell type as the cell to be evaluated.

In one embodiment, the baseline level of Fibulin-5 expression or biological activity is established in an autologous control sample obtained from the patient. The autologous control sample can be a sample of isolated cells, a tissue sample or a bodily fluid sample, and is preferably a cell sample or tissue sample. According to the present invention, and as used in the art, the term “autologous” means that the sample is obtained from the same patient from which the sample to be evaluated is obtained. The control sample should be of or from the same cell type and preferably, the control sample is obtained from the same organ, tissue or bodily fluid as the sample to be evaluated, such that the control sample serves as the best possible baseline for the sample to be evaluated. In one embodiment, when the goal of the assay is diagnosis of abnormal cell growth, it is desirable to take the control sample from a population of cells, a tissue or a bodily fluid which is believed to represent a “normal” cell, tissue, or bodily fluid, or at a minimum, a cell or tissue which is least likely to be undergoing or potentially be predisposed to develop tumor cell growth. For example, if the sample to be evaluated is an area of apparently abnormal cell growth, such as a tumorous mass, the control sample is preferably obtained from a section of apparently normal tissue (i.e., an area other than and preferably a reasonable distance from the tumorous mass) in the tissue or organ where the tumorous mass is growing. In one aspect, if a tumor to be evaluated is in the colon, the test sample would be obtained from the suspected tumor mass and the control sample would be obtained from a different section of the colon, which is separate from the area where the mass is located and which does not show signs of uncontrolled cellular proliferation.

In another embodiment, when the goal is to monitor tumor cell growth in the patient, the autologous baseline sample is typically a previous sample from the patient which was taken from an apparent or confirmed tumorous mass, and/or from apparently normal (i.e., non-tumor) tissue in the patient (or a different type of baseline for normal can be used, as discussed below). Therefore, a second method for establishing a baseline level of Fibulin-5 expression or biological activity is to establish a baseline level of Fibulin-5 expression or biological activity from at least one measurement of Fibulin-5 expression or biological activity in a previous sample from the same patient. Such a sample is also an autologous sample, but is taken from the patient at a different time point than the sample to be tested. Preferably, the previous sample(s) were of a same cell type, tissue type or bodily fluid type as the sample to be presently evaluated. In one embodiment, the previous sample resulted in a negative diagnosis (i.e., no tumor cell growth, or potential therefor, was identified). In this embodiment, a new sample is evaluated periodically (e.g., at annual physicals), and as long as the patient is determined to be negative for tumor development, an average or other suitable statistically appropriate baseline of the previous samples can be used as a “negative control” for subsequent evaluations. For the first evaluation, an alternate control can be used, as described below, or additional testing may be performed to confirm an initial negative diagnosis, if desired, and the value for Fibulin-5 expression or biological activity can be used thereafter. This type of baseline control is frequently used in other clinical diagnosis procedures where a “normal” level may differ from patient to patient and/or where obtaining an autologous control sample at the time of diagnosis is either not possible, not practical or not beneficial. For example, for a patient who has periodic mammograms, the previous mammograms serve as baseline controls for the mammary tissue of the individual patient. Similarly, for a patient who is regularly screened for prostate cancer by evaluation of levels of prostate cancer antigen (PCA), previous PCA levels are frequently used as a baseline for evaluating whether the individual patient experiences a change.

In another embodiment, the previous sample from the patient resulted in a positive diagnosis (i.e., tumor growth was positively identified). In this embodiment, the baseline provided by the previous sample is effectively a positive control for tumor growth, and the subsequent samplings of the patient are compared to this baseline to monitor the progress of the tumor growth and/or to evaluate the efficacy of a treatment which is being prescribed for the cancer. In this embodiment, it may also be beneficial to have a negative baseline level of Fibulin-5 expression or biological activity (i.e., a normal cell baseline control), so that a baseline for remission or regression of the tumor can be set. Monitoring of a patient's tumor growth can be used by the clinician to modify cancer treatment for the patient based on whether an increase or decrease in cell growth is indicated.

It will be clear to those of skill in the art that some samples to be evaluated will not readily provide an obvious autologous control sample, or it may be determined that collection of autologous control samples is too invasive and/or causes undue discomfort to the patient. In these instances, an alternate method of establishing a baseline level of Fibulin-5 expression or biological activity can be used, examples of which are described below.

Another method for establishing a baseline level of Fibulin-5 expression or biological activity is to establish a baseline level of Fibulin-5 expression or biological activity from control samples, and preferably control samples that were obtained from a population of matched individuals. It is preferred that the control samples are of the same sample type as the sample type to be evaluated for Fibulin-5 expression or biological activity (e.g., the same cell type, and preferably from the same tissue or organ). According to the present invention, the phrase “matched individuals” refers to a matching of the control individuals on the basis of one or more characteristics which are suitable for the type of cell or tumor growth to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). For example, levels of Fibulin-5 expression in the breast of a normal individual (i.e., having breast tissue that is not neoplastically transformed or predisposed to such transformation) may be higher in individuals of a given classification (e.g., elderly vs. teenagers, smokers vs. non-smokers) (although such variation in groups is not currently known). To establish a control or baseline level of Fibulin-5 expression or biological activity, samples from a number of matched individuals are obtained and evaluated for Fibulin-5 expression or biological activity. The sample type is preferably of the same sample type and obtained from the same organ, tissue or bodily fluid as the sample type to be evaluated in the test patient. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values.

A baseline such as that described above, can be a negative control baseline, such as a baseline established from a population of apparently normal control individuals. Alternatively, as discussed above, such a baseline can be established from a population of individuals that have been positively diagnosed as having cancer, and particularly, cancer of a specified stage, as set forth by the medical community, so that one or more baseline levels can be established for use in staging a cancer in the patient to be evaluated. Therefore, in one embodiment, the baseline level is one or more tumor control samples that is correlated with a particular stage of tumor development for that type of tumor. For example, tumor samples from an appropriate number of individuals which have been diagnosed as having a particular stage of a given cancer (e.g., Stage I colon cancer) are tested for Fibulin-5 expression or biological activity. The values obtained from these control samples are statistically processed to establish a suitable baseline level using methods standard in the art for establishing such values, and the baseline is noted as being indicative of that particular stage of cancer. Preferably, a similar value is determined for each of the established stages of the given cancer, so that a panel of baseline values, each representing a different stage of the cancer, is formed. The level of Fibulin-5 expression or biological activity in the patient sample is then compared to each of the baseline levels to determine to which baseline the Fibulin-5 level of the patient is statistically closest. It will be appreciated that a given patient sample may fall between baseline levels of two different stages such that the best diagnosis is that the patient tumor is at least at the lower stage, but is perhaps in the process of advancing to the higher stage. The data provided by this method can be used in conjunction with current cancer staging methods to assist the physician in the evaluation of the patient and in prescribing suitable treatment for the cancer.

It will be appreciated by those of skill in the art that a baseline need not be established for each assay as the assay is performed but rather, a baseline can be established by referring to a form of stored information regarding a previously determined baseline level of Fibulin-5 expression for a given control sample, such as a baseline level established by any of the above-described methods. Such a form of stored information can include, for example, but is not limited to, a reference chart, listing or electronic file of population or individual data regarding “normal” (negative control) or tumor positive (including staged tumors) Fibulin-5 expression; a medical chart for the patient recording data from previous evaluations; or any other source of data regarding baseline Fibulin-5 expression that is useful for the patient to be diagnosed.

After the level of Fibulin-5 expression or biological activity is detected in the sample to be evaluated for tumor cell growth, such level is compared to the established baseline level of Fibulin-5 expression or biological activity, determined as described above. Also, as mentioned above, preferably, the method of detecting used for the sample to be evaluated is the same or qualitatively and/or quantitatively equivalent to the method of detecting used to establish the baseline level, such that the levels of the test sample and the baseline can be directly compared. In comparing the test sample to the baseline control, it is determined whether the test sample has a measurable decrease or increase in Fibulin-5 expression or biological activity over the baseline level, or whether there is no statistically significant difference between the test and baseline levels. After comparing the levels of Fibulin-5 expression or biological activity in the samples, the final step of making a diagnosis, monitoring, or staging of the patient can be performed as discussed above.

For the majority of cell types, detection of a decreased level of Fibulin-5 expression or biological activity in the sample to be evaluated (i.e., the test sample) as compared to the baseline level indicates that, as compared to the baseline sample, increased tumorigenicity or a potential therefore is indicated in the cells corresponding to the test sample. This indication of increased tumorigenicity is evaluated based on what the baseline represents, and can mean: (1) a positive diagnosis of tumorigenicity (i.e., neoplastic transformation) or potential for tumor cell growth in the patient; (2) continued or increased tumorigenicity in a patient previously diagnosed with a cancer; and/or (3) a higher stage of tumorigenicity than that represented by the baseline. More specifically, if the baseline is a normal or negative control sample (i.e., autologous or otherwise established, such as from a population control), a detection of decreased Fibulin-5 expression or biological activity in the test sample as compared to the control sample indicates that the cells in the test sample are undergoing (or are at risk of undergoing) increased, and likely inappropriate (i.e., tumorous, neoplastic) cell growth. If the baseline sample is a previous sample from the patient (or a population control) and is representative of a positive diagnosis of tumor cell growth in the patient (i.e., a positive control), a detection of decreased Fibulin-5 expression or biological activity in the sample as compared to the baseline indicates that the cells in the test sample are experiencing increased tumor growth or a potential therefor, which would suggest to a clinician that a treatment currently being prescribed, for example, is not controlling the tumor growth or that tumor growth in the patient has recurred. If the baseline sample is representative of a particular stage of tumor, a detection of decreased Fibulin-5 expression or biological activity in the sample as compared to the baseline indicates that the cells in the test sample are at a higher stage of tumor growth than the stage represented by the baseline sample (e.g., if the baseline represented a stage I breast tumor, the test sample is likely to be higher than stage I, and should be compared to a stage II, III or IV baseline). As discussed above, for a minority of cell types, such as fibroblasts, the inverse scenario is indicated. For example, if the test cell type is a fibroblast, detection of a decreased level of Fibulin-5 expression or biological activity in the fibroblast test sample as compared to the baseline level indicates that, as compared to the baseline sample, decreased tumorigenicity or a potential therefore is indicated in the test cells. One of skill in the art, given the guidance provided herein, will readily be able to determine the Fibulin-5 profile for normal and tumor cells of a given cell type, and then use the method of the present invention accordingly.

Similarly, for the majority of cell types, detection of an increased level of Fibulin-5 expression or biological activity in the sample to be evaluated (i.e., the test sample) as compared to the baseline level indicates that, as compared to the baseline sample, decreased tumorigenicity or a potential therefore is indicated in the test cells. This indication of decreased tumorigenicity is evaluated based on what the baseline represents, and can mean: (1) a negative diagnosis of tumorigenicity (neoplastic transformation) or potential for tumor cell growth in the patient; (2) reduced tumorigenicity in a patient previously diagnosed with a cancer; and/or (3) a lower stage of tumorigenicity than that represented by the baseline. More specifically, if the baseline is a normal or negative control (autologous or otherwise established, such as from a population control), a detection of increased Fibulin-5 expression or biological activity in the test sample as compared to the control sample indicates that the cells in the test sample are also normal and are not predicted to be at risk of undergoing inappropriate (i.e., tumorous, neoplastic) cell growth. If the baseline sample is a previous sample from the patient (or from a population control) and is representative of a positive diagnosis of tumorigenicity in the patient (i.e., a positive control), a detection of increased Fibulin-5 expression or biological activity in the sample as compared to the baseline indicates that the cells in the test sample are experiencing decreased tumorigenicity or a potential therefor, which suggests to a clinician, for a patient that has cancer, that a treatment currently being prescribed, for example, is successfully controlling the tumor growth or that a tumor in the patient is in remission or eliminated. If the baseline sample is representative of a particular stage of tumor, a detection of increased Fibulin-5 expression or biological activity in the sample as compared to the baseline indicates that the cells in the test sample are at a lower stage of tumor growth than the stage represented by the baseline sample (e.g., if the baseline represented a stage II breast tumor, the test sample is likely to be lower than stage I, and should be compared to a stage I and negative (normal) baseline).

Finally, detection of Fibulin-5 expression that is not statistically significantly different than the Fibulin-5 expression or biological activity in the baseline sample indicates that, as compared to the baseline sample, no difference in tumorigenicity or a potential therefore is indicated in the test cells. This indication of effectively a “baseline level” of cell growth in the test cell is evaluated based on what the baseline represents, and can mean: (1) a negative or positive diagnosis of tumorigenicity (neoplastic transformation) or potential therefore in the patient; (2) unchanged tumorigenicity in a patient previously diagnosed with a cancer; and/or (3) a correlation with a stage of tumor growth that is represented by the baseline. More specifically, if the baseline is a normal or negative control (autologous or otherwise established, such as from a population control), a detection of Fibulin-5 expression or biological activity in the test sample that is not statistically significantly different than the baseline sample indicates that the cells in the test sample are also normal and are not predicted to be at risk of undergoing inappropriate (i.e., tumorous, neoplastic) cell growth. If the baseline sample is a previous sample from the patient (or from a population control) and is representative of a positive diagnosis of tumor cell growth in the patient (i.e., a positive control), a detection of Fibulin-5 expression or biological activity in the sample that is not statistically significantly different than the baseline indicates that the cells in the test sample are experiencing tumor cell growth or a potential therefor, and the patient should be further evaluated for cancer. In a patient who has cancer and is being monitored for tumor progression, a detection of Fibulin-5 expression or biological activity in the test sample that is not statistically significantly different than the baseline sample indicates that the tumor is neither increasing (progressing) or decreasing (regressing). Such a diagnosis might suggest to a clinician that a treatment currently being prescribed, for example, is ineffective in controlling the tumor growth. Finally, if the baseline sample is representative of a particular stage of tumor, a detection of Fibulin-5 expression or biological activity in the test sample that is not statistically significantly different than the baseline sample indicates that the cells in the test sample are at substantially the same stage of tumor growth as the stage represented by the baseline sample.

As discussed above, a positive diagnosis indicates that increased cell growth, and possibly tumor cell growth (neoplastic transformation), has occurred, is occurring, or is statistically likely to occur in the cells or tissue from which the sample was obtained. In order to establish a positive diagnosis, the level of Fibulin-5 activity is modulated (increased or decreased, depending on the cell or tissue type) over the established baseline by an amount that is statistically significant (i.e., with at least a 95% confidence level, or p<0.05). Preferably, detection of at least about a 10% change in Fibulin-5 expression or biological activity in the sample as compared to the baseline level results in a positive diagnosis of increased cell growth for said sample, as compared to the baseline. More preferably, detection of at least about a 30% change in Fibulin-5 expression or biological activity in the sample as compared to the baseline level results in a positive diagnosis of increased cell growth for said sample, as compared to the baseline. More preferably, detection of at least about a 50% change, and more preferably at least about a 70% change, and more preferably at least about a 90% change, or any percentage change between 5% and higher in 1% increments (i.e., 5%, 6%, 7%, 8% . . . ) in Fibulin-5 expression or biological activity in the sample as compared to the baseline level results in a positive diagnosis of increased tumorigenicity for said sample. In one embodiment, a 1.5 fold change in Fibulin-5 expression or biological activity in the sample as compared to the baseline level results in a positive diagnosis of increased tumorigenicity for said sample. More preferably, detection of at least about a 3 fold change, and more preferably at least about a 6 fold change, and even more preferably, at least about a 12 fold change, and even more preferably, at least about a 24 fold change, or any fold change from 1.5 up in increments of 0.5 fold (i.e., 1.5, 2.0, 2.5, 3.0 . . . ) in Fibulin-5 expression or biological activity as compared to the baseline level, results in a positive diagnosis of increased tumorigenicity for said sample.

As discussed in detail above, for the majority of cell types, if the level of Fibulin-5 expression or biological activity in the test sample is less than the baseline level of Fibulin-5 expression or biological activity (with statistical significance as described above), then a positive diagnosis of increased tumorigenicity, as compared to the baseline, is indicated. Similarly, for the majority of cell types, if the level of Fibulin-5 expression or biological activity in the test sample is greater than the baseline level of Fibulin-5 expression or biological activity (with statistical significance as described above), then a negative diagnosis of decreased tumorigenicity, as compared to the baseline, is indicated. The inverse is true for the minority of cell types, which can be determined as discussed previously herein. As discussed above, a negative diagnosis typically refers to a determination that neoplastic transformation has not occurred in the cells or tissue from which the sample was obtained and that there is no indication that neoplastic transformation is or will occur in such cells as of the time the evaluation is performed, or that reduced tumorigenicity is occurring in the cells or tissue from which the sample was obtained as compared to the baseline. A negative diagnosis may be used in future evaluations to establish a negative baseline for the patient and/or be used to assist with the establishment of a population control level when combined with results from other patients considered to be normal. Finally, if the level of Fibulin-5 expression or biological activity in the test sample is statistically significantly the same as the baseline level of Fibulin-5 expression or biological activity (using the confidence levels set forth above), then the test cells are believed to be experiencing substantially the same cell growth as the baseline sample, and the diagnosis is dependent on what the baseline sample represents (i.e., a positive or negative control, or a stage of tumor development). It will be appreciated that in any embodiment, the final evaluation of what is indicated by a change in Fibulin-5 expression or biological activity as compared to the baseline, beyond the established indication of increased, decreased, or unchanged cell growth, is dependent upon what the baseline represents.

In one embodiment, a positive diagnosis of neoplastic transformation in a sample obtained from a patient can be indicative of the development, or potential for development, of neoplastic transformation of the cell type, tissue and/or organ from which the sample was obtained. For example, a positive diagnosis in a sample obtained from the breast is indicative of breast cancer, or the potential therefor, in the patient. Once a positive diagnosis is made using the present method, the diagnosis can be substantiated, if desired, using any suitable alternate method of detection of tumor cell growth, including pathology screening, blood screening for tumor antigens, and surgery. In one embodiment of the present invention, the method can include an additional step of confirming the diagnosis of tumor cell growth using such an alternate form of detection of neoplastic transformation such as surgery, tumor antigen screening, biopsy and/or pathology/histology. A positive diagnosis of tumor cell growth in an individual allows for the commencement of appropriate treatment protocols. Since the method of the present invention is useful for the early detection of inappropriate cell growth in an individual, treatment protocols are expected to be more effective and result in prolonged survival rates.

Yet another embodiment of the present invention relates to an assay kit for diagnosing tumor cell growth or a potential for tumor cell growth in a patient. The assay kit includes: (a) a means for detecting Fibulin-5 expression or activity in a test sample; and (b) a means for detecting a control marker characteristic of a cell type in the test sample.

This assay kit, and the diagnostic/monitoring method of the present invention are believed to be highly useful for the detection and monitoring of a variety of tumor types. Other diagnostic assays described prior to the present invention may rely on markers which are not necessarily present in all patients that have or are at risk of developing tumors (i.e., genetic markers that are predictive of only a subset of cancer patients, such as BRACI for breast cell tumors). Moreover, such markers are typically detected as an “all or nothing” response, and therefore provide only a “yes or no” answer and are not useful for staging tumors, for example. In contrast, the method of the present invention can be used for the detection of tumorigenicity or a potential therefore in any cell type that expresses Fibulin-5, regardless of whether other genetic markers have predisposed an patient to the cancer. As discussed in the Examples, Fibulin-5 is expressed in a large variety of tissue types. Moreover, the method of the present invention is designed to test for varying levels of Fibulin-5 expression and/or biological activity as a marker of neoplastic transformation, and therefore provides more than a “yes/no” answer in that tumor development in a patient can be staged using the assay kit and method of the present invention. Therefore, the assay kit and diagnostic method of the present invention are believed to be significantly more powerful and useful than previously described tumor assays.

According to the present invention, a means for detecting Fibulin-5 expression or biological activity can be any suitable reagent which can be used in a method for detection of Fibulin-5 expression or biological activity as described previously herein. Such reagents include, but are not limited to: a probe that hybridizes under stringent hybridization conditions to a nucleic acid molecule encoding Fibulin-5 or a fragment thereof (including to a Fibulin-5-specific regulatory region in the Fibulin-5-encoding gene); RT-PCR primers for amplification of mRNA encoding Fibulin-5 or a fragment thereof; and/or an antibody, antigen-binding fragment thereof or other antigen-binding peptide that selectively binds to Fibulin-5.

According to the present invention, a probe is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. As used herein, stringent hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

PCR primers are also nucleic acid sequences, although PCR primers are typically oligonucleotides of fairly short length which are used in polymerase chain reactions. PCR primers and hybridization probes can readily be developed and produced by those of skill in 25 the art, using sequence information from the target sequence. (See, for example, Sambrook et al., supra or Glick et al., supra).

Antibodies that selectively bind to Fibulin-5 in the sample can be produced using Fibulin-5 protein information available in the art. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.). Antibodies useful in the assay kit and methods of the present invention can include polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as antigen binding partners or antigen binding peptides, that have been designed to bind selectively to the protein of interest (Fibulin-5). Examples of the design of such polypeptides, which possess a prescribed ligand specificity are given in Beste et al. (Proc. Natl. Acad. Sci. 96:1898-1903, 1999), incorporated herein by reference in its entirety.

The means for detecting a control marker characteristic of the cell type that is being sampled can generally be any type of reagent that can be used in a method of detecting the presence of a known marker in a sample, such as by a method for detecting the presence of Fibulin-5 described previously herein. Specifically, the means is characterized in that it identifies a specific marker of the cell type being analyzed that positively identifies the cell type. For example, in a breast tumor assay, it is desirable to screen breast epithelial cells for the level of Fibulin-5 expression and/or biological activity. Therefore, the means for detecting a control marker identifies a marker that is characteristic of an epithelial cell and preferably, a breast epithelial cell, so that the cell is distinguished from other cell types, such as a fibroblast. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The means for detecting of part (a) and or part (b) of the assay kit of the present invention can be conjugated to a detectable tag or detectable label. Such a tag can be any suitable tag which allows for detection of the means of part (a) or (b) and includes, but is not limited to, any composition or label detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

In addition, the means for detecting of part (a) and or part (b) of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly effecting the activity and/or ability of the detection means to detect the desired target molecule. Exemplary organic solid supports include polymers such as polystyrene, nylon, phenol-formaldehyde resins, acrylic copolymers (e.g., polyacrylamide), stabilized intact whole cells, and stabilized crude whole cell/membrane homogenates. Exemplary biopolymer supports include cellulose, polydextrans (e.g., Sephadex®), agarose, collagen and chitin. Exemplary inorganic supports include glass beads (porous and nonporous), stainless steel, metal oxides (e.g., porous ceramics such as ZrO₂, TiO₂, Al₂O₃, and NiO) and sand.

According to the present invention, the method and assay for assessing the tumorigenicity of cells in a patient, as well as other methods disclosed herein, are suitable for use in a patient that is a member of the Vertebrate class, Mammalia, including, without limitation, primates, livestock and domestic pets (e.g., a companion animal). Most typically, a patient will be a human patient.

Another embodiment of the present invention relates to a method to identify a compound useful for the inhibition of tumor cell growth or malignancy. Such a method includes the steps of: (a) detecting an initial level of Fibulin-5 expression or activity in a tumor cell or soluble sample or product derived from the tumor cell (e.g., cell supernate); (b) contacting the tumor cell with a test compound; (c) detecting a level of Fibulin-5 expression or activity in the tumor cell (or sample derived therefrom) after contact of the tumor cell with the compound; and, (d) selecting a compound that changes the level of Fibulin-5 expression or activity in the tumor cell, as compared to the initial level of Fibulin-5 expression or activity, toward a baseline level of Fibulin-5 expression or activity established from a non-tumor cell, wherein the selected compound is predicted to be useful for inhibition of tumor growth or malignancy. The method can include a further step of detecting whether a compound selected in (d) inhibits the growth or characteristics of malignancy (neoplastic transformation) of a tumor cell.

Steps (a) and (c) of the method of the present invention require detection of Fibulin-5 expression and/or biological activity in a tumor cell or in a sample derived from the tumor cell, such as a cellular extract or supernate. Detection of Fibulin-5 expression and/or biological activity can include, but is not limited to: detecting Fibulin-5 mRNA transcription (e.g., by polymerase chain reaction (PCR), reverse transcriptase-PCR (RT-PCR), in situ hybridization, Northern blot, sequence analysis or detection of a reporter gene); detecting Fibulin-5 translation (e.g., by immunoblot, enzyme-linked immunosorbant assay (ELISA), radioimmunoassay (RIA), immunoprecipitation, immunohistochemistry and immunofluorescence); and/or detecting Fibulin-5 biological activity (e.g., by detecting any of the activities of Fibulin-5 as described elsewhere herein or as known in the art). Such methods for detection of Fibulin-5 expression and biological activity have been discussed in detail above with regard to the method for assessing tumorigenicity of cells, and that discussion applies to the detection Fibulin-5 expression and biological activity here. The step of detection in step (a) is the control level of Fibulin-5 expression or biological activity for a tumor-positive cell to which the detection in step (c) is to be compared and evaluated. The step of detection in step (c) is the experimental level of Fibulin-5 expression or biological activity which indicates whether the test compound can change the level of Fibulin-5 expression or biological activity in the cell, as compared to the level determined in step (a) and as compared to a baseline level of expression or activity that is established for a non-tumor cell. In other words, the assay determines whether a given compound is capable of changing Fibulin-5 expression or activity from a tumor phenotype to or toward a non-tumor phenotype. The baseline level of Fibulin-5 activity or expression established for a non-tumor cell can be determined by any of the methods as discussed previously herein for establishing a baseline level.

This reverse of this assay could also be performed using a normal, non-tumor cell (or sample derived therefrom), to identify compounds that change Fibulin-5 expression or activity to or toward a tumor type. Such an assay could be a valuable assay to screen for putative carcinogens.

A cell suitable for use in the present method is any cell which expresses or can be induced to express, a detectable level of Fibulin-5. A detectable level of Fibulin-5 is a level which can be detected using any of the methods for Fibulin-5 detection described herein. Since Fibulin-5 is expressed by many mammalian cell types, a variety of cell types could be selected. However, it will be appreciated by those of skill in the art that some cell types are more suitable for use in an in vitro assay (e.g., easy to maintain in culture, easy to obtain), and that Fibulin-5 may be more readily detectable in some cell types, and therefore, such cell types are preferable for use in the present invention. A preferred cell type to use in the method of the present invention is any cell type that has a high expression or low expression of Fibulin-5 in the tumor cell as compared to a non-tumor cell of the same cell type, so that a change in Fibulin-5 expression or activity is readily detectable. As discussed above, one can also use a sample derived from such a cell, such as a cell extract or cell supernate. Some preferred cells to use in the method of the present invention include, but are not limited to: fibroblasts (and fibrosarcomas), epithelial cells, and breast, colon, kidney, ovarian or uterine tumor cells. In one embodiment, a cell suitable for use in the present method is a cell which has been transfected with a recombinant nucleic acid molecule encoding Fibulin-5 and operatively linked to a transcription control sequence so that Fibulin-5 is expressed by the cell. As discussed above, the nucleic acid sequence for human Fibulin-5, as well as other mammalian Fibulin-5 sequences, are known in the art. Methods and reagents for preparing recombinant cells are known in the art.

As used herein, the term “putative regulatory compound” refers to compounds having an unknown or previously unappreciated regulatory activity in a particular process. The above-described method for identifying a compound of the present invention includes a step of contacting a test cell with a compound being tested for its ability to increase the expression or biological activity of Fibulin-5. For example, test cells can be grown in liquid culture medium or grown on solid medium in which the liquid medium or the solid medium contains the compound to be tested. In addition, as described above, the liquid or solid medium contains components necessary for cell growth, such as assimilable carbon, nitrogen and micronutrients.

The above described methods, in one aspect, involve contacting cells with the compound being tested for a sufficient time to allow for interaction of the putative regulatory compound with an element that affects Fibulin-5 expression and/or biological activity in a cell. Such elements can include, but are not limited to: a nucleic acid molecule encoding Fibulin-5 (including regulatory regions of such a molecule), Fibulin-5 protein, Fibulin-5 inhibitors, Fibulin-5 stimulators, and Fibulin-5 substrates. The period of contact with the compound being tested can be varied depending on the result being measured, and can be determined by one of skill in the art. For example, for binding assays, a shorter time of contact with the compound being tested is typically suitable, than when activity or expression is assessed. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which growth is continuing (in the case of a cell based assay) prior to scoring. The incubation time for growth of cells can vary but is sufficient to allow for the upregulation or downregulation of Fibulin-5 expression or biological activity in a cell. It will be recognized that shorter incubation times are preferable because compounds can be more rapidly screened. A preferred incubation time is between about 1 hour to about 48 hours.

The conditions under which the cell or cell lysate of the present invention is contacted with a putative regulatory compound, such as by mixing, are any suitable culture or assay conditions and includes an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) comprises between about 1 nM to about 10 mM of putative regulatory compound(s) per well of a 96-well plate.

In one aspect, the present method also makes use of non-cell based assay systems to identify compounds that can regulate Fibulin-5 expression or biological activity and thereby are predicted to be useful for regulating cell growth. For example, Fibulin-5 proteins and nucleic acid molecules encoding Fibulin-5 may be recombinantly expressed and utilized in non-cell based assays to identify compounds that bind to the protein or nucleic acid molecule, respectively. In non-cell based assays the recombinantly expressed Fibulin-5 or nucleic acid encoding Fibulin-5 is attached to a solid substrate such as a test tube, microtiter well or a column, by means well known to those in the art.

In one embodiment, DNA encoding a reporter molecule can be linked to a regulatory element of the Fibulin-5 gene (or a gene encoding a protein that directly regulates Fibulin-5) and used in appropriate intact cells, cell extracts or lysates to identify compounds that modulate Fibulin-5 gene expression, respectively. Appropriate cells or cell extracts are prepared from any cell type that normally expresses Fibulin-5, thereby ensuring that the cell extracts contain the transcription factors required for in vitro or in vivo transcription. The screen can be used to identify compounds that modulate the expression of the reporter construct. In such screens, the level of reporter gene expression is determined in the presence of the test compound and compared to the level of expression in the absence of the test compound.

Following steps (a), (b) and (c) of the present method is a step (d) of selecting a compound that changes the level of Fibulin-5 expression or activity in the tumor cell (or normal cell), as compared to the initial level of Fibulin-5 expression or activity, toward a baseline level of Fibulin-5 expression or activity established from a non-tumor cell (or tumor cell, if the test cell is a tumor cell). In other words, compounds which cause a change in the level of Fibulin-5 expression or biological activity in a tumor cell as detected in step (c) as compared to the level detected in step (a), toward the established baseline level for a non-tumor cell, are selected by the present method as being compounds that are predicted to be useful for the inhibition of carcinogenicity. Compounds that cause a change in the level of Fibulin-5 expression or biological activity in a non-tumor cell as detected in step (c) as compared to the level detected in step (a), toward the established baseline level for a tumor cell, are selected by the present method as being compounds that are predicted to be potential carcinogens.

Preferably, compounds which are selected in step (d) are compounds for which, after the test cell was contacted with the compound in step (b), the level of Fibulin-5 expression or biological activity detected in step (c) was statistically significantly (i.e., with at least a 95% confidence level, or p<0.05) changed as compared to the initial level of Fibulin-5 expression or biological activity detected in step (a). Preferably, detection of at least about a 30% change in Fibulin-5 expression or biological activity in the cell as compared to initial level results in selection of the compound according to step (d). More preferably, detection of at least about a 50% change and more preferably at least about a 70% change, and more preferably at least about a 90% change, or any percentage change between 5% and higher in 1% increments (i.e., 5%, 6%, 7%, 8% . . . ) in Fibulin-5 expression or biological activity in the cell as compared to the initial level results in selection of the compound according to step (d). In one embodiment, a 1.5 fold change in Fibulin-5 expression or biological activity in the cell as compared to the initial level results in selection of the compound according to step (d). More preferably, detection of at least about a 3 fold change, and more preferably at least about a 6 fold change, and even more preferably, at least about a 12 fold change, and even more preferably, at least about a 24 fold change, or any fold change from 1.5 up in increments of 0.5 fold (i.e., 1.5, 2.0, 2.5, 3.0 . . . ) in Fibulin-5 expression or biological activity as compared to the initial level, results in selection of the compound according to step (d).

It is to be understood that either of steps (a) and (c) of detection can result in no detection of Fibulin-5 expression or biological activity or detection of Fibulin-5. More specifically, since the level of Fibulin-5 expression or biological activity in step (a) (i.e., the initial level) is one of the baseline or control levels of Fibulin-5 for the assay, if step (a) reveals no detectable Fibulin-5 expression or biological activity, then any detectable level of Fibulin-5 expression or biological activity in step (c) is considered to be a positive result and indicative of increased Fibulin-5 activity in the cell and the appropriate assessment associated with this result. If the initial level of Fibulin-5 expression or biological activity in step (a) is a detectable level, then the level of Fibulin-5 expression or biological activity detected in step (c) is evaluated to determine whether it is statistically significantly greater than that of step (a). It is possible that the level of Fibulin-5 expression or biological activity in step (c) could be no detectable level, which would indicate that the compound did not increase Fibulin-5 activity. In this scenario, however, it should be determined that the test cell can display an increase in Fibulin-5 expression or biological activity under some conditions (i.e., by contact with a compound known to increase Fibulin-5 activity in the test cell), so that false negatives are not identified.

In one embodiment of this method of the present invention, the method further includes the step of detecting whether the compound selected in step (d) can inhibit carcinogenicity or a characteristic thereof. In this embodiment, the test cell is contacted with the compound as in step (b), and the growth characteristics of the cell before and after contact with the cell are evaluated. Evaluation of cell growth can be by any suitable method in the art, including, but not limited to, proliferation assays (e.g., by measuring uptake of [³H]-thymidine, viewing cells morphologically) and/or evaluating markers of cell growth (e.g., measurement of changes in cell surface markers, measurement of intracellular indicators of cell growth). Such methods are known in the art and are exemplified in the Examples section.

Compounds suitable for testing and use in the methods of the present invention include any known or available proteins, nucleic acid molecules, as well as products of drug design, including peptides, oligonucleotides, carbohydrates and/or synthetic organic molecules. Such an agent can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See for example, Maulik et al., 1997, Molecular Biotechnology: Therapeutic Applications and Strategies, Wiley-Liss, Inc., which is incorporated herein by reference in its entirety. Candidate compounds initially identified by drug design methods can be screened for the ability to modulate the expression and/or biological activity of Fibulin-5 using the methods described herein.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands against a desired target, and then optimize the lead molecules by either random or directed design strategies. Methods of molecular diversity are described in detail in Maulik, et al., supra.

In a rational drug design procedure, the three-dimensional structure of a regulatory compound can be analyzed by, for example, nuclear magnetic resonance (NMR) or X-ray crystallography. This three-dimensional structure can then be used to predict structures of potential compounds, such as potential regulatory agents by, for example, computer modeling. The predicted compound structure can be used to optimize lead compounds derived, for example, by molecular diversity methods. In addition, the predicted compound structure can be produced by, for example, chemical synthesis, recombinant DNA technology, or by isolating a mimetope from a natural source (e.g., plants, animals, bacteria and fungi).

Various other methods of structure-based drug design are disclosed in Maulik et al., 1997, supra. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Compounds identified by the method described above can be used in a method to regulate cell growth, as described below and any such compounds are encompassed for use in the method described below.

Yet another embodiment of the invention relates to methods to increase or decrease the expression or biological activity of Fibulin-5 in cells (e.g., isolated cells, cells of a tissue, cells in a patient) in order to achieve a goal, including, reduction of angiogenesis in a tissue, decreased tumorigenicity of tumor cells, or reduction in the potential for development of tumor cells. Such a method includes the step of increasing or decreasing the expression and/or biological activity of Fibulin-5, as required for a given cell type, in order to achieve the desired result (e.g., inhibition of angiogenesis or inhibition of tumorigenicity). Preferably, the cell in which tumorigenicity is inhibited is a cell which, prior to the application of the present method, is exhibiting inappropriate (malignant) cell growth or a potential therefor. Preferred cells to regulate according to the present invention include tumor cells. Cells in which it is desirable to inhibit tumorigenicity or tissues in which inhibition of angiogenesis is desired can be identified, for example, using the method for assessing tumorigenicity or Fibulin-5 expression and activity of the present invention as described in detail above. Such methods are particularly useful in patients where increased tumorigenicity or angiogenesis is, or predicted to become, problematic. Therefore, such a method is particularly useful to treat patients that have, or are at a risk of developing, tumor cell growth (i.e., a cancer), or to treat any other patients having a condition characterized by undesirable cell growth (e.g., lymphoproliferative disorders). Other diseases and conditions in which inhibition of tumorigenicity or angiogenesis would be desirable will be apparent to those of skill in the art and are intended to be encompassed by the present invention.

The method of the present invention includes a step of modulating (i.e., upregulating or downregulating) Fibulin-5 expression and/or biological activity in a patient that has, or is at risk of developing, inappropriate or unregulated cell growth or angiogenesis. Modulating Fibulin-5 expression or biological activity according to the present invention can be accomplished by directly affecting Fibulin-5 expression (transcription or translation) or biological activity, or by directly affecting the ability of a regulator (inhibitor or stimulator) of Fibulin-5 to bind to Fibulin-5 or to activate Fibulin-5. Preferably, the method of the present invention is targeted to a particular type of cell or tissue or region of the body in which inhibition of cell growth is desired. A targeted cell, for example, could include a tumor cell, wherein the method does not substantially affect Fibulin-5 expression or biological activity in non-tumor cells, or in cells of a different type that the tumor cell type. Therefore, the method of the present invention is intended to be specifically targeted to Fibulin-5 expression and/or biological activity for the purpose of inhibiting cell growth or inhibiting angiogenesis by modulating Fibulin-5 expression and/or biological activity.

An increase in Fibulin-5 expression and/or biological activity is defined herein as any measurable (detectable) increase (i.e., upregulation, stimulation, enhancement) of the expression or activity of Fibulin-5. As used herein, to increase Fibulin-5 expression and/or biological activity refers to any measurable increase in Fibulin-5 expression and/or biological activity by any suitable method of measurement. A decrease in Fibulin-5 expression and/or biological activity is defined herein as any measurable (detectable) decrease (i.e., downregulation, inhibition, reduction) of the expression or activity of Fibulin-5. As used herein, to decrease Fibulin-5 expression and/or biological activity refers to any measurable decrease in Fibulin-5 expression and/or biological activity by any suitable method of measurement.

Accordingly, one embodiment of the present invention includes the use of a variety of agents (i.e., regulatory compounds) which, by acting directly on Fibulin-5 (or the gene encoding Fibulin-5) or on inhibitors or stimulators of Fibulin-5, modulate (regulate up or down) the expression and/or biological activity of Fibulin-5 in a cell to produce a desired effect (e.g., inhibition of tumorigenesis). Agents useful in the present invention include, for example, proteins, nucleic acid molecules, antibodies, and compounds that are products of rational drug design (i.e., drugs). Such compounds can be identified using the method of identifying compounds for regulating tumor cell growth and malignancy as described above. Moreover, the expression or biological activity of Fibulin-5 in a cell can be determined using the methods described above.

Therefore, in one embodiment, the method of the present invention increases the transcription and/or the translation of Fibulin-5 by a cell in the patient that naturally expresses Fibulin-5 and that is the target for growth regulation. Methods for increasing the expression of Fibulin-5 include, but are not limited to, administering an agent that increases the expression, administering Fibulin-5 protein or a homologue or analog thereof to a patient, and/or overexpressing Fibulin-5 in the target cells of the patient. In one aspect of this embodiment, Fibulin-5 can be effectively overexpressed in a cell by increasing the activity of a Fibulin-5 gene promoter in the cell such that expression of endogenous Fibulin-5 in the cell is increased. For example, the activity of the Fibulin-5 gene promoter can be increased by methods which include, contacting the promoter with a transcriptional activator, inhibiting a Fibulin-5 inhibitor, and increasing the activity of a Fibulin-5 stimulator. Methods by which such compounds (e.g., transcriptional activators) can be administered to a cell are described below. In another embodiment, Fibulin-5 activity is increased by administering Fibulin-5 or a homologue or analogue (synthetic homologue or mimetic) to the target cells or to the patient in an appropriate carrier or delivery vehicle.

As used herein, reference to an isolated protein or polypeptide in the present invention, including an isolated Fibulin-5 protein, includes full-length proteins, fusion proteins, or any fragment or homologue of such a protein. Such a Fibulin-5 protein can include, but is not limited to, purified Fibulin-5 protein, recombinantly produced Fibulin-5 protein, membrane bound Fibulin-5 protein, Fibulin-5 protein complexed with lipids, soluble Fibulin-5 protein and isolated Fibulin-5 protein associated with other proteins. More specifically, an isolated protein, such as a Fibulin-5 protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated Fibulin-5 protein of the present invention is produced recombinantly. In addition, and by way of example, a “human Fibulin-5 protein” refers to an Fibulin-5 protein (generally including a homologue of a naturally occurring Fibulin-5 protein) from a human (Homo sapiens) or to an Fibulin-5 protein that has been otherwise produced from the knowledge of the structure (e.g., sequence) and perhaps the function of a naturally occurring Fibulin-5 protein from Homo sapiens. In other words, a human Fibulin-5 protein includes any Fibulin-5 protein that has substantially similar structure and function of a naturally occurring Fibulin-5 protein from Homo sapiens or that is a biologically active (i.e., has biological activity) homologue of a naturally occurring Fibulin-5 protein from Homo sapiens as described in detail herein. As such, a human Fibulin-5 protein can include purified, partially purified, recombinant, mutated/modified and synthetic proteins. According to the present invention, the terms “modification” and “mutation” can be used interchangeably, particularly with regard to the modifications/mutations to the amino acid sequence of Fibulin-5 (or nucleic acid sequences) described herein. An isolated protein useful as an antagonist or agonist according to the present invention can be isolated from its natural source, produced recombinantly or produced synthetically.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

According to the present invention, an isolated Fibulin-5 protein, including a biologically active homologue or fragment thereof, has at least one characteristic of biological activity of activity a wild-type, or naturally occurring Fibulin-5 protein (which can vary depending on whether the homologue or fragment is an agonist, antagonist, or mimic of Fibulin-5). Biological activity of Fibulin-5 and methods of determining the same have been described previously herein.

As used herein, the phrase “Fibulin-5 agonist” refers to any compound that is characterized by the ability to agonize (e.g., stimulate, induce, increase, enhance, or mimic) the biological activity of a naturally occurring Fibulin-5 as described herein, and includes any Fibulin-5 homologue, binding protein (e.g., an antibody), agent that interacts with Fibulin-5, or any suitable product of drug/compound/peptide design or selection which is characterized by its ability to agonize (e.g., stimulate, induce, increase, enhance) the biological activity of a naturally occurring Fibulin-5 protein in a manner similar to the natural agonist, Fibulin-5. Similarly, the phrase, “Fibulin-5 antagonist” refers to any compound which inhibits (e.g., antagonizes, reduces, decreases, blocks, reverses, or alters) the effect of an Fibulin-5 agonist as described above. More particularly, an Fibulin-5 antagonist is capable of acting in a manner relative to Fibulin-5 activity, such that the biological activity of the natural agonist Fibulin-5, is decreased in a manner that is antagonistic (e.g., against, a reversal of, contrary to) to the natural action of Fibulin-5. Such antagonists can include, but are not limited to, a protein, peptide, or nucleic acid (including ribozymes and antisense) or product of drug/compound/peptide design or selection that provides the antagonistic effect.

Homologues of Fibulin-5, including peptide and non-peptide agonists and antagonists of Fibulin-5 (analogues), can be products of drug design or selection and can be produced using various methods known in the art. Such homologues can be referred to as mimetics. Mimetics have been described in detail above.

In one embodiment, a Fibulin-5 homologue comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to a naturally occurring Fibulin-5 amino acid sequence.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Sch\ddot{aa}ffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

In one aspect of this embodiment of the present invention, the expression and/or biological activity of Fibulin-5 is increased by overexpressing Fibulin-5 in the cell in which growth is to be regulated. Overexpression of Fibulin-5 refers to an increase in expression of Fibulin-5 over a normal, endogenous level of Fibulin-5 expression. For some cell types, which do not express detectable levels of Fibulin-5 under normal conditions, such expression can be any detectable level. For cell types which do express detectable levels of Fibulin-5 under normal conditions, an overexpression is any statistically significant increase in expression of Fibulin-5 (p<0.05) (or constitutive expression where expression is normally not constitutive) over endogenous levels of expression. One method by which Fibulin-5 overexpression can be achieved is by transfecting the cell with a recombinant nucleic acid molecule encoding Fibulin-5 operatively linked to a transcription control sequence, wherein the recombinant Fibulin-5 is expressed by the cell. As discussed previously herein, the nucleic acid sequence encoding Fibulin-5, vectors suitable for expressing such a molecule, and methods of transfection of a cell with such a molecule, including in vivo methods, are known and are described in detail below.

A recombinant nucleic acid molecule expressing Fibulin-5 is a molecule that can include at least one of any nucleic acid sequence encoding a protein having Fibulin-5 biological activity operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

Preferably, a recombinant nucleic acid molecule is produced using recombinant DNA technology (e.g., polymerase chain reaction (PCR) amplification, cloning). Suitable nucleic acid sequences encoding Fibulin-5 for use in a recombinant nucleic acid molecule of the present invention include any nucleic acid sequence that encodes a Fibulin-5 having Fibulin-5 biological activity and suitable for use in the target host cell. For example, when the target host cell is a human cell, human Fibulin-5-encoding nucleic acid sequences are preferably used, although the present invention is not limited to strict use of naturally occurring sequences or same-species sequences.

Knowing the nucleic acid sequences of certain nucleic acid molecules of the present invention allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways including traditional cloning techniques using oligonucleotide probes to screen appropriate libraries or DNA and PCR amplification of appropriate libraries or DNA using oligonucleotide primers. Preferred libraries to screen or from which to amplify nucleic acid molecule include mammalian genomic DNA libraries. Techniques to clone and amplify genes are disclosed, for example, in Sambrook et al., ibid.

A recombinant nucleic acid molecule includes a recombinant vector, which is any nucleic acid sequence, typically a heterologous sequence, which is operatively linked to the isolated nucleic acid molecule encoding a Fibulin-5 protein, which is capable of enabling recombinant production of the Fibulin-5 protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules. Recombinant vectors are preferably used in the expression of nucleic acid molecules, and can also be referred to as expression vectors. Preferred recombinant vectors are capable of being expressed in a transfected host cell, and particularly, in a transfected mammalian host cell in vivo.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more transcription control sequences. The phrase “operatively linked” refers to linking a nucleic acid molecule to a transcription control sequence in a manner such that the molecule is able to be expressed when transfected (i.e., transformed, transduced or transfected) into a host cell.

Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in a host cell according to the present invention. A variety of suitable transcription control sequences are known to those skilled in the art. Preferred transcription control sequences include those which function in mammalian cells, with cell- or tissue-specific transcription control sequences being particularly preferred. Examples of preferred transcription control sequences include, but are not limited to, transcription control sequences useful for expression of a protein in breast epithelial cells and tumor cells and the naturally occurring Fibulin-5 promoter. Particularly preferred transcription control sequences include inducible promoters, cell-specific promoters, tissue-specific promoters (e.g., insulin promoters) and enhancers. Suitable promoters for these and other cell types will be easily determined by those of skill in the art. Transcription control sequences of the present invention can also include naturally occurring transcription control sequences naturally associated with the protein to be expressed prior to isolation. In one embodiment, a transcription control sequence includes an inducible promoter.

One type of recombinant vector useful in a recombinant nucleic acid molecule of the present invention is a recombinant viral vector. Such a vector includes a recombinant nucleic acid sequence encoding a Fibulin-5 protein of the present invention that is packaged in a viral coat that can be expressed in a host cell in an animal or ex vivo after administration. A number of recombinant viral vectors can be used, including, but not limited to, those based on alphaviruses, poxviruses, adenoviruses, herpesviruses, lentiviruses, adeno-associated viruses and retroviruses. Particularly preferred viral vectors are those based on adenoviruses and adeno-associated viruses. Viral vectors suitable for gene delivery are well known in the art and can be selected by the skilled artisan for use in the present invention. A detailed discussion of current viral vectors is provided in “Molecular Biotechnology,” Second Edition, by Glick and Pasternak, ASM Press, Washington D.C., 1998, pp. 555-590, the entirety of which is incorporated herein by reference.

For example, a retroviral vector, which is useful when it is desired to have a nucleic acid sequence inserted into the host genome for long term expression, can be packaged in the envelope protein of another virus so that it has the binding specificity and infection spectrum that are determined by the envelope protein (e.g., a pseudotyped virus). In addition, the envelope gene can be genetically engineered to include a DNA element that encodes and amino acid sequence that binds to a cell receptor to create a recombinant retrovirus that infects a specific cell type. Expression of the gene (i.e., the Fibulin-5 gene) can be further controlled by the use of a cell or tissue-specific promoter. Retroviral vectors have been successfully used to transfect cells with a gene which is expressed and maintained in a variety of ex vivo systems

An adenoviral vector is a preferred vector for use in the present method. An adenoviral vector infects a wide range of human cells and has been used extensively in live vaccines. Adenoviral vectors used in gene therapy do not integrate into the host genome, and therefore, gene therapy using this system requires periodic administration, although methods have been described which extend the expression time of adenoviral transferred genes, such as administration of antibodies directed against T cell receptors at the site of expression (Sawchuk et al., 1996, Hum. Gene. Ther. 7:499-506). The efficiency of adenovirus-mediated gene delivery can be enhanced by developing a virus that preferentially infects a particular target cell. For example, a gene for the attachment fibers of adenovirus can be engineered to include a DNA element that encodes a protein domain that binds to a cell-specific receptor. Examples of successful in vivo delivery of genes has been demonstrated and is discussed in more detail below.

Yet another type of viral vector is based on adeno-associated viruses, which are small, nonpathogenic, single-stranded human viruses. This virus can integrate into a specific site on chromosome 19. This virus can carry a cloned insert of about 4.5 kb, and has typically been successfully used to express proteins in vivo from 70 days to at least 5 months. Demonstrating that the art is quickly advancing in the area of gene therapy, however, a recent publication by Bennett et al. reported efficient and stable transgene expression by adeno-associated viral vector transfer in vivo for greater than 1 year (Bennett et al., 1999, Proc. Natl. Acad Sci. USA 96:9920-9925).

Another type of viral vector that is suitable for use in the present invention is a herpes simplex virus vector. Herpes simplex virus type 1 infects and persists within nondividing neuronal cells, and is therefore a suitable vector for targeting and transfecting cells of the central and peripheral nervous system with a Fibulin-5 protein of the present invention. Preclinical trials in experimental animal models with such a vector has demonstrated that the vector can deliver genes to cells of both the brain and peripheral nervous system that are expressed and maintained for long periods of time.

Suitable host cells to transfect with a recombinant nucleic acid molecule according to the present invention include any mammalian cell that can be transfected. Host cells can be either untransfected cells or cells that are already transfected with at least one nucleic acid molecule. Host cells according to the present invention can be any cell capable of producing a Fibulin-5 protein as described herein or in which it is desired to produce Fibulin-5.

According to the present invention, a host cell can also be referred to as a target cell or a targeted cell in vivo, in which a recombinant nucleic acid molecule encoding a Fibulin-5 protein having Fibulin-5 biological activity is to be expressed. As used herein, the term “target cell” or “targeted cell” refers to a cell to which a recombinant nucleic acid molecule of the present invention is selectively designed to be delivered. The term target cell does not necessarily restrict the delivery of a recombinant nucleic acid molecule only to the target cell and no other cell, but indicates that the delivery of the recombinant molecule, the expression of the recombinant molecule, or both, are specifically directed to a preselected host cell. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety). Ways in which viral vectors can be modified to deliver a nucleic acid molecule to a target cell have been discussed above. Alternatively, the route of administration, as discussed below, can be used to target a specific cell or tissue. For example, intracoronary administration of an adenoviral vector has been shown to be effective for the delivery of a gene cardiac myocytes (Maurice et al., 1999, J. Clin. Invest. 104:21-29). Intravenous delivery of cholesterol-containing cationic liposomes has been shown to preferentially target pulmonary tissues (Liu et al., Nature Biotechnology 15:167, 1997), and effectively mediate transfer and expression of genes in vivo. Other examples of successful targeted in vivo delivery of nucleic acid molecules are known in the art. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

According to the method of the present invention, a host cell is preferably transfected in vivo (i.e., in a mammal) as a result of administration to a mammal of a recombinant nucleic acid molecule, or ex vivo, by removing cells from a mammal and transfecting the cells with a recombinant nucleic acid molecule ex vivo. Transfection of a nucleic acid molecule into a host cell according to the present invention can be accomplished by any method by which a nucleic acid molecule administered into the cell in vivo, and includes, but is not limited to, transfection, electroporation, microinjection, lipofection, adsorption, viral infection, naked DNA injection and protoplast fusion. Methods of administration are discussed in detail below.

In one embodiment of the present invention, a recombinant nucleic acid molecule of the present invention is administered to a patient in a liposome delivery vehicle, whereby the nucleic acid sequence encoding the Fibulin-5 protein enters the host cell (i.e., the target cell) by lipofection. A liposome delivery vehicle contains the recombinant nucleic acid molecule and delivers the molecules to a suitable site in a host recipient. According to the present invention, a liposome delivery vehicle comprises a lipid composition that is capable of delivering a recombinant nucleic acid molecule of the present invention, including both plasmids and viral vectors, to a suitable cell and/or tissue in a patient. A liposome delivery vehicle of the present invention comprises a lipid composition that is capable of fusing with the plasma membrane of the target cell to deliver the recombinant nucleic acid molecule into a cell.

A liposome delivery vehicle of the present invention can be modified to target a particular site in a mammal (i.e., a targeting liposome), thereby targeting and making use of a nucleic acid molecule of the present invention at that site. Suitable modifications include anipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands).

A liposome delivery vehicle is preferably capable of remaining stable in a patient for a sufficient amount of time to deliver a nucleic acid molecule of the present invention to a preferred site in the patient (i.e., a target cell). A liposome delivery vehicle of the present invention is preferably stable in the patient into which it has been administered for at least about 30 minutes, more preferably for at least about 1 hour and even more preferably for at least about 24 hours. A preferred liposome delivery vehicle of the present invention is from about 0.01 microns to about 1 microns in size.

Suitable liposomes for use with the present invention include any liposome. Preferred liposomes of the present invention include those liposomes commonly used in, for example, gene delivery methods known to those of skill in the art. Preferred liposome delivery vehicles comprise multilamellar vesicle (MLV) lipids and extruded lipids. Methods for preparation of MLV's are well known in the art and are described, for example, in the Examples section. According to the present invention, “extruded lipids” are lipids which are prepared similarly to MLV lipids, but which are subsequently extruded through filters of decreasing size, as described in Templeton et al., 1997, Nature Biotech., 15:647-652, which is incorporated herein by reference in its entirety. Small unilamellar vesicle (SUV) lipids can also be used in the composition and method of the present invention. In one embodiment, liposome delivery vehicles comprise liposomes having a polycationic lipid composition (i.e., cationic liposomes) and/or liposomes having a cholesterol backbone conjugated to polyethylene glycol. In a preferred embodiment, liposome delivery vehicles useful in the present invention comprise one or more lipids selected from the group of DOTMA, DOTAP, DOTIM, DDAB, and cholesterol.

Preferably, the transfection efficiency of a nucleic acid:liposome complex of the present invention is at least about 1 picogram (pg) of protein expressed per milligram (mg) of total tissue protein per microgram (μg) of nucleic acid delivered. More preferably, the transfection efficiency of a nucleic acid:liposome complex of the present invention is at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered.

Complexing a liposome with a nucleic acid molecule of the present invention can be achieved using methods standard in the art. A suitable concentration of a nucleic acid molecule of the present invention to add to a liposome includes a concentration effective for delivering a sufficient amount of recombinant nucleic acid molecule into a target cell of a patient such that the Fibulin-5 protein encoded by the nucleic acid molecule can be expressed in a an amount effective to inhibit the growth of the target cell. Preferably, from about 0.1 μg to about 10 μg of nucleic acid molecule of the present invention is combined with about 8 nmol liposomes. In one embodiment, the ratio of nucleic acids to lipids (μg nucleic acid:nmol lipids) in a composition of the present invention is preferably at least from about 1:10 to about 6:1 nucleic acid:lipid by weight (i.e., 1:10=1 μg nucleic acid:10 nmol lipid).

According to the present invention, a regulatory compound for increasing the expression or biological activity of Fibulin-5, including a recombinant nucleic acid molecule encoding Fibulin-5, is typically administered to a patient in a composition. In addition to the recombinant nucleic acid molecule or other Fibulin-5 regulatory compound (i.e., a protein, antibody, carbohydrate, small molecule product of drug design), the composition can include, for example, a pharmaceutically acceptable carrier, which includes pharmaceutically acceptable excipients and/or delivery vehicles, for delivering the recombinant nucleic acid molecule or other regulatory compound to a patient (e.g., a liposome delivery vehicle). As used herein, a pharmaceutically acceptable carrier refers to any substance suitable for delivering a therapeutic composition useful in the method of the present invention to a suitable in vivo or ex vivo site. Preferred pharmaceutically acceptable carriers are capable of maintaining a recombinant nucleic acid molecule of the present invention in a form that, upon arrival of the nucleic acid molecule to a target cell, the nucleic acid molecule is capable of entering the cell and being expressed by the cell. Suitable excipients of the present invention include excipients or formularies that transport or help transport, but do not specifically target a nucleic acid molecule to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into an animal. As used herein, a controlled release formulation comprises recombinant nucleic acid molecule or other Fibulin-5 regulatory compound of the present invention in a controlled release vehicle. Suitable controlled release vehicles include, but are not limited to, biocompatible polymers, other polymeric matrices, capsules, microcapsules, microparticles, bolus preparations, osmotic pumps, difflusion devices, liposomes, lipospheres, and transdermal delivery systems. Suitable delivery vehicles have been previously described herein, and include, but are not limited to liposomes, viral vectors or other delivery vehicles, including ribozymes. Natural lipid-containing delivery vehicles include cells and cellular membranes. Artificial lipid-containing delivery vehicles include liposomes and micelles. As discussed above, a delivery vehicle of the present invention can be modified to target to a particular site in a patient, thereby targeting and making use of a nucleic acid molecule at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

As discussed above, a composition of the present invention is administered to a patient in a manner effective to deliver the recombinant nucleic acid molecule comprising a nucleic acid sequence encoding a Fibulin-5 protein having Fibulin-5 biological activity to a target cell, whereby the target cell is transfected by the recombinant molecule and whereby the Fibulin-5 protein is expressed in the target cell. When another Fibulin-5 regulatory compound is to be delivered to a target cell in a patient, the composition is administered in a manner effective to deliver the Fibulin-5 regulatory compound to the target cell, whereby the compound can act on the cell (e.g., enter the cell and act on Fibulin-5 or an inhibitor or stimulator thereof) so that Fibulin-5 expression or biological activity is increased. Suitable administration protocols include any in vivo or ex vivo administration protocol.

According to the present invention, an effective administration protocol (i.e., administering a composition of the present invention in an effective manner) comprises suitable dose parameters and modes of administration that result in transfection and expression of a recombinant nucleic acid molecule encoding a Fibulin-5 protein or an other Fibulin-5 regulatory compound, in a target cell of a patient, and subsequent inhibition of the growth of the target cell, preferably so that the patient obtains some measurable, observable or perceived benefit from such administration. In some situations, where the target cell population is accessible for sampling, effective dose parameters can be determined using methods as described herein for assessment of tumor growth. Such methods include removing a sample of the target cell population from the patient prior to and after the recombinant nucleic acid molecule is administered, and measuring changes in Fibulin-5 expression or biological activity, as well as measuring inhibition of the cell. Alternatively, effective dose parameters can be determined by experimentation using in vitro cell cultures, in vivo animal models, and eventually, clinical trials if the patient is human. Effective dose parameters can be determined using methods standard in the art for a particular disease or condition that the patient has or is at risk of developing. Such methods include, for example, determination of survival rates, side effects (i.e., toxicity) and progression or regression of disease.

According to the present invention, suitable methods of administering a composition comprising a recombinant nucleic acid molecule of the present invention to a patient include any route of in vivo administration that is suitable for delivering a recombinant nucleic acid molecule into a patient. The preferred routes of administration will be apparent to those of skill in the art, depending on the type of delivery vehicle used, the target cell population, whether the compound is a protein, nucleic acid, or other compound (e.g., a drug) and the disease or condition experienced by the patient. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor. For example, when the tumor is a breast tumor, the preferred methods of administration include impregnation of a catheter, and direct injection into the tumor.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a recombinant nucleic acid molecule to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety. For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. Dow et al. also demonstrated successful in vivo delivery of a nucleic acid molecule by direct injection into a site of a tumor. As discussed above, Liu et al., 1997, ibid. demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N.Y Acad. Sci. 811:299-308; and von der Leyen et al., 1995, Proc Natl Acad Sci USA 92:1137-1141).

As discussed above, delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al., 1999, ibid. demonstrated that an adenoviral vector encoding a β2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572).

In the area of neuronal gene delivery, multiple successful in vivo gene transfers have been reported. Millecamps et al. reported the targeting of adenoviral vectors to neurons using neuron restrictive enhancer elements placed upstream of the promoter for the transgene (phosphoglycerate promoter). Such vectors were administered to mice and rats intramuscularly and intracerebrally, respectively, resulting in successful neuronal-specific transfection and expression of the transgene in vivo (Millecamps et al., 1999, Nat. Biotechnol. 17:865-869). As discussed above, Bennett et al. reported the use of adeno-associated viral vector to deliver and express a gene by subretinal injection in the neural retina in vivo for greater than 1 year (Bennett, 1999, ibid.).

Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti-T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a Fibulin-5 encoding recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include a nucleic acid molecule of the present invention, and preferably includes a recombinant molecule of the present invention that preferably is replication, or otherwise amplification, competent. A naked nucleic acid reagent of the present invention can comprise one or more nucleic acid molecule of the present invention in the form of, for example, a dicistronic recombinant molecule. Naked nucleic acid delivery can include intramuscular, subcutaneous, intradermal, transdermal, intranasal and oral routes of administration, with direct injection into the target tissue being most preferred. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized and/or topically. In one embodiment, pure DNA constructs cover the surface of gold particles (1 to 3 μm in diameter) and are propelled into skin cells or muscle with a “gene gun.”

In accordance with the present invention, a suitable single dose of a recombinant nucleic acid molecule encoding a Fibulin-5 protein as described herein is a dose that is capable of transfecting a host cell and being expressed in the host cell at a level sufficient, in the absence of the addition of any other factors or other manipulation of the host cell, to inhibit the growth of the host cell when administered one or more times over a suitable time period. Doses can vary depending upon the cell type being targeted, the route of administration, the delivery vehicle used, and the disease or condition being treated.

In one embodiment, an appropriate single dose of a nucleic acid:liposome complex of the present invention is from about 0.1 μg to about 100 μg per kg body weight of the patient to which the complex is being administered. In another embodiment, an appropriate single dose is from about 1 μg to about 10 μg per kg body weight. In another embodiment, an appropriate single dose of nucleic acid:lipid complex is at least about 0.1 μg of nucleic acid, more preferably at least about 1 μg of nucleic acid, even more preferably at least about 10 μg of nucleic acid, even more preferably at least about 50 μg of nucleic acid, and even more preferably at least about 100 μg of nucleic acid.

Preferably, an appropriate single dose of a recombinant nucleic acid molecule encoding a Fibulin-5 protein of the present invention results in at least about 1 pg of protein expressed per mg of total tissue protein per μg of nucleic acid delivered. More preferably, an appropriate single dose is a dose which results in at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered.

When the Fibulin-5 regulatory agent is a protein, small molecule (i.e., the products 20 of drug design) or antibody, a preferred single dose of such a compound typically comprises between about 0.01 microgram×kilogram⁻¹ and about 10 milligram×kilograms⁻¹ body weight of an animal. A more preferred single dose of an agent comprises between about 1 microgram×kilograms⁻¹ and about 10 milligram×kilogram⁻¹ body weight of an animal. An even more preferred single dose of an agent comprises between about 5 microgram×kilogram⁻¹ and about 7 milligram×kilogram⁻¹ body weight of an animal. An even more preferred single dose of an agent comprises between about 10 microgram×kilogram⁻¹ and about 5 milligram×kilogram⁻¹ body weight of an animal. Another particularly preferred single dose of an agent comprises between about 0.1 microgram×kilograms⁻¹ and about 10 microgram×kilogram⁻¹ body weight of an animal, if the agent is delivered parenterally.

In one embodiment of the present invention, it is desirable to decrease Fibulin-5 expression or activity in a cell. In this embodiment, it is desired to modify a target cell in order to decrease in Fibulin-5 gene expression, decrease the function of the gene, or decrease the function of the gene product (i.e., the protein encoded by the gene). Such methods can be referred to as inactivation (complete or partial), deletion, interruption, blockage or down-regulation of a gene encoding Fibulin-5. In one embodiment, reduction in Fibulin-5 activity or expression is achieved by use of a Fibulin-5 antagonist, which is any compound which inhibits (e.g., antagonizes, reduces, decreases, blocks, reverses, or alters) the effect of Fibulin-5. Such antagonists can include, but are not limited to, a protein, peptide, or nucleic acid (including ribozymes and antisense) or product of drug/compound/peptide design or selection that provides the antagonistic effect.

According to the present invention, a ribozyme typically contains stretches of complementary RNA bases that can base-pair with a target RNA ligand, including the RNA molecule itself, giving rise to an active site of defined structure that can cleave the bound RNA molecule (See Maulik et al., 1997, supra). Therefore, a ribozyme can serve as a targeting delivery vehicle for a nucleic acid molecule, or alternatively, the ribozyme can target and bind to RNA encoding Fibulin-5, for example, and thereby effectively inhibit the translation of Fibulin-5.

As used herein, an anti-sense nucleic acid molecule is defined as an isolated nucleic acid molecule that reduces expression of a Fibulin-5 protein by hybridizing under high stringency conditions to a gene encoding the Fibulin-5 protein. Such a nucleic acid molecule is sufficiently similar to the nucleic acid sequence encoding the Fibulin-5 protein that the molecule is capable of hybridizing under high stringency conditions to the coding strand of the gene or RNA encoding the natural Fibulin-5 protein. In a particularly preferred embodiment, an anti-sense nucleic acid molecule of the present invention is the exact complement of the coding region of a Fibulin-5 protein or of a regulatory region of a gene encoding the Fibulin-5 protein. It is noted that the anti-sense of the coding region does not necessarily include the anti-sense of the stop codon.

In another embodiment, a targeting vector can be used to deliver a particular nucleic acid molecule into a recombinant host cell, wherein the nucleic acid molecule is used to delete or inactivate an endogenous gene (e.g., Fibulin-5-encoding gene) within the host cell or microorganism (i.e., used for targeted gene disruption or knock-out technology). Such a vector may also be known in the art as a “knock-out” vector. In one aspect of this embodiment, a portion of the vector, but more typically, the nucleic acid molecule inserted into the vector (i.e., the insert), has a nucleic acid sequence that is homologous to a nucleic acid sequence of a target gene in the host cell (i.e., a gene which is targeted to be deleted or inactivated). The nucleic acid sequence of the vector insert is designed to bind to the target gene such that the target gene and the insert undergo homologous recombination, whereby the endogenous target gene is deleted, inactivated or attenuated (i.e., by at least a portion of the endogenous target gene being mutated or deleted).

Compositions of the present invention can be administered to any mammalian patient, and preferably to humans. According to the present invention, administration of a composition is useful to inhibit the tumorigenicity of a target cell or to inhibit angiogenesis in a tissue of a patient. Typically, it is desirable to inhibit the growth of a target cell to obtain a therapeutic benefit in the patient. Patients whom are suitable candidates for the method of the present invention include, but are not limited to, patients that have, or are at risk of developing (e.g., are predisposed to), cancer or a lymphoproliferative disease, or any condition in which regulation of angiogenesis might be beneficial. Increasing or decreasing Fibulin-5 expression or biological activity to inhibit tumorigenicity in the absence of obtaining some therapeutic benefit is useful for the purposes of determining factors involved (or not involved) in a disease and preparing a patient to more beneficially receive another therapeutic composition. In a preferred embodiment, however, the methods of the present invention are directed to the inhibition of tumorigenicity of a target cell or inhibition of angiogenesis in a tissue, which is useful in providing some therapeutic benefit to a patient.

As such, a therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition (e.g., metastatic tumor growth resulting from a primary cancer), and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment). In particular, protecting a patient from a disease is accomplished by inhibiting the tumorigenicity of a target cell in the patient by regulating Fibulin-5 expression or biological activity such that a beneficial effect is obtained. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

As discussed above, the present inventors have discovered that Fibulin-5 is a target for TGFβ activity. Therefore, yet another embodiment of the present invention relates to a method to identify a regulator of transforming growth factor β (TGFβ), comprising: (a) contacting a cell (or secreted product thereof) that expresses TGFβ and Fibulin-5 with a putative regulatory compound; (b) detecting the expression of Fibulin-5 in the cell (or secreted product); and (c) comparing the expression of Fibulin-5 after contact with the compound to the expression of Fibulin-5 before contact with the compound, wherein detection of a change in the expression of Fibulin-5 in the cells after contact with the compound as compared to before contact with the compound indicates that the compound is a putative regulator of TAFβ. In other words, Fibulin-5 expression or activity is used as a surrogate marker or endpoint for evaluating TGFβ activity in a cell. Many aspects of this embodiment of the invention are similar to those described above for a method to identify a compound that regulates tumorigenicity or malignancy of a cell, such as various steps of contacting the cell with a compound, culturing a cell, and/or detecting and evaluating Fibulin-5 activity. Such techniques and methods are encompassed by this method of the invention as well.

As used herein, the term “putative regulatory compound” or “putative regulatory ligand” refers to compounds having an unknown regulatory activity, at least with respect to the ability of such compounds to regulate progesterone receptors as described herein.

In the method of identifying a regulatory compound (i.e., an agonist or an antagonist) of TGFβ activity according to the present invention, the method can be a cell-based assay, or non-cell-based assay. In one embodiment, the conditions under which a cell that expresses Fibulin-5 is contacted with a putative regulatory ligand, such as by mixing, are conditions in which TGFβ activity is not stimulated (activated) if essentially no regulatory compound is present. For example, such conditions include normal culture conditions in the absence of a known TGFβ stimulatory compound. The putative regulatory compound is then contacted with the cell or cell lysate (if a non-cell based assay). In this embodiment, the step of detecting is designed to indicate whether the putative regulatory compound alters the biological activity of the TGFβ as compared to in the absence of the putative regulatory compound (i.e., the background level), as determined by the effects of the contact between the compound and the cell on the expression or activity of Fibulin-5.

In an alternate embodiment, the conditions under which a cell or cell lysate is contacted with a putative regulatory compound, such as by mixing, are conditions in which TGFβ is normally stimulated (activated) if essentially no regulatory compound is present. Such conditions can include, for example, contact of the cell with a TGFβ stimulator molecule. In this embodiment, the putative regulatory compound can be contacted with the cell or cell lysate prior to, or simultaneously with, the contact of the cell or cell lysate with the stimulatory compound, or after contact of the cell with the stimulatory compound (e.g., to determine whether the putative regulatory compound downregulates, or reduces the activation of TGFβ).

Cells that are useful in the cell-based assays of the present invention include any cell that expresses TGFβ and Fibulin-5, which include a variety of naturally occurring cell types from a variety of tissue types, as discussed previously herein. Cells suitable for use in a cell-based assay include normal or malignant cells, as well as cells that are not malignant, but which are abnormal, such as cells from a non-malignant tissue that is otherwise diseased.

According to the present invention, the method includes the step of detecting the expression or activity of Fibulin-5. To detect expression of Fibulin-5 refers to the act of actively determining whether the gene encoding Fibulin-5 or whether the Fibulin-5 protein is expressed or not. This can include determining whether the gene or protein expression is upregulated as compared to a control, downregulated as compared to a control, or unchanged as compared to a control. Therefore, the step of detecting expression does not require that expression of Fibulin-5 is actually upregulated or downregulated, but rather, can also include detecting that the expression of the Fibulin-5 gene or protein has not changed (i.e., detecting no expression of the gene or protein or no change in expression of the gene or protein). Methods to detect Fibulin-5 expression or activity have been described in detail above, as have methods of comparing one level of expression or activity to another.

The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

The following examples are provided for the purpose of illustration and are not intended to limit the scope of the present invention.

### EXAMPLES

The following materials and methods were used in Examples 1-7 below.

Recombinant human TGF-β1 was kindly provided by R&D Systems. The cDNA constructs encoding dominant-negative versions of FLAG-tagged Smad3/3A (pMX-Smad3/3A-IRES-GFP) and HA-tagged MKK1 (pMCL-MKK1-8E) were generously provided by Drs. Xeudong Liu (University of Colorado) and Natalie Ahn (University of Colorado), respectively. The FLAG-tagged dominant-negative p38 MAPKα [pcDNA3-p3 8MAPKα(KM)] and AP-1-luciferase (pAP-1-luciferase; Stratagene) plasmids were kindly provided by Dr. Yi Qun Xiao (National Jewish Medical and Research Center), while the cyclin A-luciferase construct (pCAL2-luciferase) was generously supplied by Dr. Rik Derynck (UCSF). Human dermal microvascular HMEC-1 endothelial cells were kindly provided by Drs. Edwin Ames, Thomas Lawley, and Francisco Candal (CDC). All additional supplies or reagents were routinely available.

Fibulin-5 Plasmids. Full-length human and murine FBLN-5 cDNAs were PCR amplified from ESTs H17726 and AW106432, respectively. The resulting PCR fragments were engineered to contain unique Hind III (N-terminus) and Sac II (C-terminus) restriction sites for subcloning into the corresponding sites in pcDNA3.1/Myc-His B vector (In Vitrogen) to C-terminally tag FBLN-5 with a Myc- and (His)₆-tag.

A retroviral FBLN-5 vector was synthesized by PCR amplifying the full-length Myc-His-tagged murine FBLN-5 cDNA using oligonucleotides containing Xho I (N-terminus) and Eco RI (C-terminus) restriction sites. Afterwards, the resulting PCR product was ligated into identical sites located immediately upstream of the IRES in the bicistronic retroviral vector, pMSCV-IRES-GFP (11).

All FBLN-5 cDNA inserts were sequenced on an Applied Biosystems 377A DNA sequencing machine.

Mass Spectrometry. Murine 3T3-L1 fibroblasts were plated onto 10-cm dishes and cultured in Dulbecco's modified Eagle's medium (DMEM) supplemented with 10% fetal bovine serum. Upon reaching ˜90% confluency, the cells were washed extensively in PBS and serum-starved in DMEM for 12 hr at 37° C. Quiescent 3T3-L1 cells were then metabolically labeled with [³⁵S]methionine in the absence or presence of TGF-β1 for 12 hr at 37° C. Afterwards, naïve- and TGF-β-CDM were collected, clarified by centrifugation, and concentrated by acetone precipitation prior to fractionation through 15% SDS-PAGE. Secreted proteins were visualized by silver staining and autoradiography of the dried gel. A differentially expressed ˜60 kDa protein that was evident in TGF-β-CDM was excised from the gel and subjected to trypsin digestion as described previously (12). The resulting peptides were sprayed from nanoelectrospray needles supplied by Protana A/S (Odense, Denmark) and analyzed on a Micromass q-Tof mass spectrometer (Manchester, UK) essentially as described earlier (13). Data acquisition and analysis were performed on a Mass Lynx Windows NT PC data system.

Northern Blotting. Quiescent 3T3-L1 cells and human dermal microvascular HMEC-1 endothelial cells were stimulated with TGF-β1 as above. Afterwards, total RNA was isolated using the RNAzol B reagent (Tel-Test) and 10 μg was subsequently fractionated through 1.2% agarose/formaldehyde gels. After immobilizing to nylon membrane, the RNA was probed with a ³²P-labeled human FBLN-5 cDNA probe (nucleotides 887-1437) for 60 min at 68° C. After hybridization, the membrane was washed for 45 min at room temperature in 2×SSC/0.05% SDS, followed by 45 min of washing at 50° C. in 0.1×SSC/0.1% SDS prior to visualization of the FBLN-5 mRNA by autoradiography.

Tissue-specific expression of FBLN-5 mRNA in humans and mice was monitored by hybridizing human and murine multiple tissue Northern blots (Clontech) and a murine embryonic Northern blot (Clontech) to ³²P-labeled species-specific FBLN-5 cDNA probes (human, nucleotides 887-1437; murine, 1056-1579). The procedures for hybridizing, washing, and visualizing FBLN-5 mRNA were identical to those above.

Retroviral infections. Phoenix retroviral packaging cells (Dr. Gary Nolan, Stanford University) were cultured on 10-cm plates and transiently transfected at 70% confluency by overnight exposure to calcium phosphate precipitate containing 10 μg of pCL-Eco (14) and 10 μg of either pMSCV-IRES-GFP or pMSCV-FBLN-5-IRES-GFP. The transfectants were fed with fresh media the following morning and retroviral supernatants were collected 48 hr later. Murine 3T3-L1 cells, human HT1080 fibrosarcoma (15), and mink lung Mv1Lu cells epithelial cells (16) were infected overnight with 5 ml of either control (i.e., pMSCV-IRES-GFP) or FBLN-5 retroviral supernates in the presence of 4 μg/ml of Polybrene. The cells were analyzed at 48 hr for GFP expression on a FACSVantage cell sorter (Becton Dickinson), and the highest 10% of GFP-expressing cells were expanded to generate stable populations that were ˜70% GFP-positive. These initial populations were subjected to a second round of GFP sorting on a MoFlo cell sorter (Cytomation), which yielded stable populations of control or FBLN-5-expressing cells with equivalent GFP levels at a positivity rate of ≧90%. These stable populations of 3T3-L1, HT1080, and Mv1Lu cells were used to analyze the effects of FBLN-5 on cell growth, motility, and invasion, as well as reporter gene expression and protein kinase activation.

[³H]Thymidine Incorporation Assays. To detect inhibitory effects on DNA synthesis, GFP- or FBLN-5-expressing cells were cultured onto 96-well plates at a density of 5,000 cells/well in complete DMEM supplemented with various concentrations of TGF-β1 (0→5 ng/ml) for 48 hr at 37° C. During the final 4 hr of stimulation, newly synthesized DNA was radiolabeled by adding [³H]thymidine to the culture media. Afterwards, the cells were washed twice in ice-cold PBS, precipitated with ice-cold 5% TCA, and subsequently solubilized in 0.5N NaOH prior to scintillation counting to determine radionucleotide incorporation into DNA.

To detect stimulatory effects on DNA synthesis, GFP- or FBLN-5-expressing cells were also cultured onto 96-well plates at a density of 5,000-10,000 cells/well, and were then incubated overnight in complete DMEM. The next day, the cells were washed twice in PBS and placed in serum-free DMEM with or without TGF-β1 as above. Cellular DNA was radiolabeled by addition of [³H]thymidine during the final 12 hr of stimulation and subsequently prepared for scintillation counting as above. Data are the mean (±SEM) of five independent experiments presented as the percent [³H]thymidine incorporation normalized to untreated GFP-expressing cells.

Tumor Array. The effects of tumorigenesis on FBLN-5 expression were examined by hybridizing a ³²P-radiolabeled full-length human FBLN-5 cDNA probe to a matched human normal/tumor cDNA array according to the manufacturer's instructions (Clontech). FBLN-5 expression in normal and malignant human tissues was subsequently visualized by autoradiography. cDNA arrays were subsequently hybridized with a ³²P-labeled ubiquitin cDNA probe provided by the manufacturer. The expression of FBLN-5 was normalized to that of ubiquitin, and the ratio of FBLN-5 expression between individual pairs of normal and tumor tissue was determined. A ratio of ≧2 or ≦0.5 was considered significant.

In Vitro Migration and Invasion Assays. The effects of FBLN-5 on the motility and invasiveness of HT1080 cells were determined using a modified Boyden-chamber assay essentially as described (17,18). Briefly, the underside of a porous membrane (8μ pore, 24-well format; Becton Dickinson) was coated overnight at 4° C. in PBS containing 50 μg/ml fibronectin (In Vitrogen). Afterwards, the fibronectin mixtures were removed and replaced with serum-free DMEM containing 0.1% BSA (SFM/0.1% BSA). Dissociated GFP- (i.e., control) or FBLN-5-expressing HT1080 cells were washed twice in SFM/0.1% BSA and subsequently cultured for 18-24 hr at 37° C. in the upper chambers at a density of 100,000 cells/well. Afterwards, the cells were washed twice in ice-cold PBS and immediately fixed for 10 min in 95% ethanol. After fixation, cells remaining in the upper chamber were removed with a cotton swab, while those remaining in the lower chamber were stained with crystal violet.

The effects of FBLN-5 on the invasiveness of HT1080 cells were determined similarly except that GFP- or FBLN-5-expressing HT1080 cells were cultured onto Matrigel-coated membranes (Becton Dickinson). Approximately 48 hr after plating, cells migrating through the synthetic basement membranes were fixed and stained with crystal violet as above.

The number of migrating or invading HT1080 cells was determined through three independent measures, all giving similar results: (i) manual counting under a light microscope; (ii) automated counting using the IPLab Spectrum Software package (Scanalytics Inc.); and (iii) extraction of crystal violet dye by incubating the membranes in 10% acetic acid, followed by spectrophotometry at 590 nm. Data are the mean (±SEM) of nine independent experiments presented as the percent migration or invasion relative to GFP-expressing HT1080 cells.

Luciferase Reporter Gene Assays. GFP- or FBLN-5-expressing Mv1Lu cells were plated onto 24-well plates at a density of 22,500 cells/well for pCAL2-luciferase, or at 45,000 cells/well for pAP-1-luciferase. The cells were transiently transfected the following day by overnight exposure to Fugene 6 (Roche) liposomes containing 350 ng/well of total DNA comprised of 200 ng of either pCAL2-luciferase or pAP-1-luciferase, 0→100 ng of either pcDNA3-p38MAPKα(KM) or pMCL-MKK1-8E, and 50 ng of pCMV-β-gal (Clontech), which was used to control for differences in transfection efficiencies. The following morning the cells were washed twice in PBS and were incubated for 18 hr in serum-free DMEM with or without TGF-β1 (5 ng/ml) for AP-1-luciferase assays, or for 48 hr in DMEM/1% FBS with or without TGF-β1 (5 ng/ml) for cyclin A-luciferase assays. Afterwards, luciferase and β-gal activities contained in detergent-solubilized cell extracts were determined. Unless otherwise indicated, data are the mean (±SEM) luciferase activities of three or more independent experiments normalized to untreated GFP-expressing cells.

Protein Kinase Activation. GFP- or FBLN-5-expressing cells were cultured onto 6-cm dishes. Upon reaching ˜90% confluency, the cells were washed twice in PBS and were serum-starved in DMEM for 60 min prior to stimulation with TGF-β1 for 0-24 hr at 37° C. Afterwards, the cells were washed twice in ice-cold PBS and lysed in 350 μl of Buffer H/1% Triton X-100 (19). After incubation on ice for 30 min, the resulting whole cell extracts were clarified by microcentrifugation. The stimulation of protein kinase activities was determined by fractionating 15-25 μg/lane of clarified whole cell extract through 10% SDS-PAGE, and then transferring them electrophoretically to nitrocellulose. Afterwards, the membrane was probed with a 1:500-1:1000 dilution of either anti-phospho-ERK1/2 or -phospho-p38 MAPK polyclonal antibodies (New England Biolabs), and the resulting immunocomplexes were visualized by enhanced chemiluminescence. Differences in protein loading were monitored by reprobing stripped membranes with a 1:1000 dilution of either anti-ERK1 or -p38 MAPK polyclonal antibodies. Immunocomplexes were visualized as above.

Alternatively, human 293T cells were cultured on 10 cm plates and transiently transfected at ˜80% confluency by overnight exposure to Fugene 6 liposomes containing 30 μg/plate of either pcDNA3.1/Myc-His or pcDNA3.1/FBLN-5/Myc-His. The ensuing morning the cells were washed twice in PBS and cultured in serum-free DMEM (6 ml) for 24-48 hr at 37° C. Afterwards, control- (i.e., empty vector transfectants) or FBLN-5-conditioned media (CDM) was collected and clarified by centrifugation at 2000 rpm for 5 min at 4° C. Aliquots (1 ml) of control- or FBLN-5-CDM were then used to stimulate quiescent 3T3-L1 cells cultured on 6-well plates for 0-30 min at 37° C. Thereafter, the activation ERK1/2 by CDM was assessed by immunoblot analysis as described above.

### Example 1

The following example demonstrates that FBLN-5 is a novel TGF-β-inducible fibroblast secretory protein.

In order to identify secretory proteins whose expression are regulated by TGF-β, proteins present in naïve- and TGF-β-conditioned media (CDM) of murine 3T3-L1 fibroblasts were concentrated and fractionated through SDS-PAGE. Differentially expressed proteins regulated by TGF˜β were excised and subjected to in situ trypsinization prior to identification by mass spectrometry. A ˜60 kDa protein was prominent in TGF-β-CDM, but not in naïve-CDM (data not shown). Sequencing of a single peptide by nanoelectrospray MS/MS analysis returned an amino acid sequence of (NH₂)—YPGAYYIFQIK—(COOH) (SEQ ID NO:1), corresponding to residues 374-384 of murine FBLN-5 (SEQ ID NO:3).

FBLN-5 is a recently identified 448 aa member of the Fibulin family (9,10). Like other Fibulins, FBLN-5 contains multiple calcium-binding EGF-like repeats (i.e., six) and a globular C-terminal domain; it also contains an integrin-binding RGD motif that binds αvβ3, αvβ5, and αvβ9 integrins (4) and mediates endothelial cell adhesion (10). Mice deficient in FBLN-5 expression are viable, but exhibit pronounced abnormalities of the skin, lung, and vasculature resulting from profound elastic fiber disorganization (i.e., elastinopathy). Thus, FBLN-5 regulates organogenesis and, like other Fibulins, is widely expressed in throughout human and murine tissues (e.g., heart, skeletal muscle, spleen, small intestine, liver, lung, kidney, placenta) (data not shown; (9,10)). Not surprisingly, the inventors found that FBLN-5 expression is induced significantly during embryogenesis (data not shown).

In order to confirm that FBLN-5 expression was induced by TGF-β and to establish the mechanism for this effect, Northern blot analysis was performed on total RNA prepared from TGF-β-treated 3T3-L1 cells. The results showed that treatment of 3T3-L1 cells with TGF-β stimulated the synthesis of FBLN-5 transcript in a time- and dose-dependent manner (data not shown). Thus, FBLN-5 is indeed a novel gene target for TGF-β in 3T3-L1 cells.

The biological actions of TGF-β are mediated primarily through its stimulation of a relatively simple signaling system that at its core is comprised of three TGF-β receptors, types I, II, and III, and three latent transcription factors, Smads 2, 3, and 4 (20,21). In order to determine whether TGF-β-mediated induction of FBLN-5 expression was Smad2/3-dependent, 3T3-L1 cells were infected with control (i.e., GFP) or dominant-negative Smad3/3A retrovirus [pMX-Smad3/3A-IRES-GFP; (16)] and cells expressing GFP were isolated by flow cytometry to establish stable polyclonal populations of control and Smad3/3A-expressing 3T3-L1 cells (data not shown). As shown in FIG. 1, stable expression of dominant-negative Smad3/3A in 3T3-L1 cells significantly inhibited their synthesis of DNA as compared to control 3T3-L1 cells. The ability of Smad3/3A to reduce 3T3-L1 cell DNA synthesis in the absence of serum or growth factors implicates autocrine TGF-β signaling as a mediator of 3T3-L1 cell growth. Accordingly, treatment of 3T3-L1 cells with TGF-β significantly stimulated their synthesis of DNA in a dose-dependent manner: this response to TGF-β was completely abrogated by overexpression of dominant-negative Smad3/3A in 3T3-L1 cells (data not shown). In contrast, overexpression of dominant-negative Smad3/3A failed to inhibit the ability of TGF-β to stimulate FBLN-5 expression in 3T3 -L1 cells (data not shown). Taken together, these findings establish FBLN-5 as a novel TGF-β-inducible gene target whose expression when stimulated by TGF-β proceeds through a Smad2/3-independent pathway.

In addition to mediating endothelial cell adhesion (10), FBLN-5 expression is induced dramatically in mechanically injured vascular endothelial and smooth muscle cells (9,10). Because TGF-β induces FBLN-5 expression, and because TGF-β regulates endothelial cell activity, it was determined whether TGF-β induces FBLN-5 expression in human dermal microvascular HMEC-1 endothelial cells. TGF-β treatment of HMEC-1 cells stimulated their transcription of FBLN-5 mRNA (data not shown). This finding indicates that the ability of TGF-β to stimulate FBLN-5 expression is not restricted to fibroblasts, but may instead represent a generalized cellular response to TGF-β. Moreover, this finding establishes FBLN-5 as a potential mediator of the effects of TGF-β on endothelial cell activity.

### Example 2

The following example shows that FBLN-5 stimulates 3T3-L1 cell proliferation and MAP kinases.

Fibulins stimulate (Fibulin-4; (22); Fibulin-3; (23)) or inhibit (Fibulin-3; (23); Fibulin-1; (18,24)) proliferation in a context- and cell type-specific manner. However, the role of FBLN-5 in regulating cell growth remains to be established. The present inventors' finding that FBLN-5 is secreted by 3T3-L1 cells suggested that FBLN-5 expression may function in governing cell growth. To test this hypothesis, 3T3 -L1 cells were infected with control (i.e., pMSCV-IRES-GFP) or FBLN-5 retrovirus and cells expressing GFP were isolated by flow cytometry to establish stable polyclonal populations of control and FBLN-5 expressing 3T3-L1 cells (data not shown). As shown in FIG. 2A, expression of FBLN-5 in 3T3-L1 cells did not affect their sensitivity to TGF-β (inset). However, in the absence of added TGF-β or growth factors, overexpression of FBLN-5 in 3T3-L1 cells significantly enhanced their synthesis of DNA as compared to control cells. Similarly, treatment of 3T3-L1 cells with recombinant FBLN-5 also stimulated their synthesis of DNA (data not shown). Thus, these data indicate that FBLN-5 is a positive regulator of fibroblast growth.

Mammalian MAP kinases (e.g., ERKs, JNKs, and p38 MAPKs) are important mediators in virtually all physiological processes, including the control of gene expression, programmed cell death, and cell proliferation (25,26). It was therefore hypothesized that FBLN-5 expression would lead to stimulation of MAP kinases. This hypothesis seemed especially attractive given that FBLN-5 binds integrins (4,10) and integrins stimulate MAP kinases (27). To test this hypothesis, GFP- or FBLN-5-expressing 3T3 -L1 cells were serum-starved for 60 min prior to stimulation with TGF-β for various lengths of time. Afterwards, the activation status of MAP kinases was determined by immunoblot analysis using phospho-specific anti-MAP kinase antibodies. When constitutively overexpressed in 3T3-L1 cells, FBLN-5 failed to affect the rate and extent of activation of p38 MAPK (FIG. 2B) or ERK1/2 (FIG. 2C) by TGF-β. However, in the absence of added TGF-β or growth factors, serum-starved FBLN-5-expressing 3T3-L1 cells exhibited significantly higher activities of p38 MAPK (FIG. 2B) and ERK1/2 (FIG. 2C) as compared to control cells. Likewise, ERK1/2 activities were greater in 3T3-L1 cells stimulated with CDM from FBLN-5-expressing 293T cells as compared to CDM from control cells (data not shown). Lastly, it is worth noting that while TGF-β treatment of3T3-L1 cells did indeed stimulate their activation of JNK, FBLN-5 expression did not promote JNK activation in response to serum starvation (data not shown). Taken together, these findings clearly show that expression of FBLN-5 in 3T3-L1 cells selectively stimulates the activation of p38 MAPK and ERK1/2, but not JNK. Moreover, these findings implicate p38 MAPK and ERK1/2 as potential mediators of the effects of TGF-β on 3T3-L1 cell proliferation.

### Example 3

The following example demonstrates that FBLN-5 expression enhances human fibrosarcoma malignancy.

The ability of FBLN-5 to promote DNA synthesis and MAP kinase activation in 3T3-L1 fibroblasts led the inventors to speculate that FBLN-5 may also function in regulating tumorigenesis. In support of this hypothesis, recent studies have established that Fibulins can either augment [Fibulin-4; (22,28)] or attenuate [Fibulin-1; (18,24)] the malignancy of cancer cells. Therefore, the role of FBLN-5 in regulating the malignancy of human HT1080 fibrosarcoma cells was examined. These cells were chosen because they lack expression of FBLN-5 (data not shown) and its relative, Fibulin-1 (18). Importantly, re-expression of Fibulin-1 in Fibulin-1-deficient fibrosarcomas negates their malignancy by inhibiting their invasion, and by preventing their growth in soft agar or mice after implantation (18).

To determine the effects of FBLN-5 on the malignancy of cancer cells, HT1080 cells that stably express the murine ecotropic receptor (15) were infected with control (i. e., GFP) or FBLN-5 retrovirus. Afterwards, cells that expressed GFP were isolated by flow cytometry to establish stable polyclonal populations of GFP control and FBLN-5-expressing HT1080 cells. The resulting HT1080 cell lines had purities≧90% and expressed GFP indistinguishably (data not shown). As expected, HT1080 cells infected with FBLN-5 retrovirus expressed and secreted high levels of FBLN-5 protein into the media, while those infected with GFP control retrovirus were negative for expression of recombinant FBLN-5 protein (data not shown). These stable populations of HT1080 cells were used to examine the effects of FBLN-5 expression on fibrosarcoma malignancy.

As shown in FIG. 3A, FBLN-5-expressing HT1080 cells exhibited a trend towards enhanced DNA synthesis as compared to control cells (p=0.11); however, TGF-β treatment in combination with FBLN-5 expression significantly stimulated DNA synthesis in HT1080 cells (FIG. 3A). Thus, similar to its effects on 3T3-L1 cell proliferation, FBLN-5 also enhances the growth of human fibrosarcoma cells.

The inventors also investigated whether FBLN-5 expression regulated HT1080 cell migration towards fibronectin. As shown in FIG. 3B, FBLN-expressing cells migrated more readily to fibronectin as compared to control HT1080 cells. Moreover, when confronted with synthetic basement membranes, FBLN-5 expressing HT1080 cells invaded significantly better than control HT1080 cells (FIG. 3C). Taken together, these findings demonstrate that FBLN-5 expression enhances the malignancy of human HT1080 fibrosarcoma cells by increasing their migration and invasion, and their production of DNA in response to TGF-β.

### Example 4

The following example demonstrates that tumorigenesis alters FBLN-5 expression in human tissues.

The present inventors' finding that FBLN-5 expression enhanced the malignancy of human fibrosarcoma cells prompted the examination of the effects of tumorigenesis on FBLN-5 expression in human tissues. To address this question, a radiolabeled human FBLN-5 cDNA probe was hybridized to a membrane arrayed with matched normal/tumor cDNAs generated from cancer patients. Of the 68 patients surveyed, FBLN-5 expression was altered in 65% (44/68) of the tumors, of which 95% (42/44) showed downregulation and 5% (2/44) upregulation (data not shown). The reduction or loss of FBLN-5 expression was especially evident in cancers of the kidney (93%; 14/15 cases), breast (100%; 9/9 cases), ovary (100%; 3/3 cases), and colon (55%;6/11 cases) (datanot shown). Importantly, FBLN-5 was expressed aberrantly in 68% of metastatic human malignancies (17/25 cases), of which 100% showed downregulated expression of FBLN-5 (data not shown). Taken together, these findings provide the first evidence that FBLN-5 expression is altered dramatically during tumorigenesis, suggesting that aberrant FBLN-5 expression may affect cancer cell growth and metastasis in a context- and/or tumor-specific manner. Moreover, despite the ability of FBLN-5 to enhance the malignancy of human fibrosarcoma cells (see Example 3), the overwhelming trend for its downregulated expression in human tumors, particularly in cancers of the kidney, breast, ovary, and colon, suggests that FBLN-5 expression normally functions as a tumor suppressor.

### Example 5

The following example demonstrates that FBLN-5 inhibits Mv1Lu cell proliferation and cyclin A expression.

Fibulin family members, including FBLN-5, are produced predominantly by fibroblasts localized to boundaries between epithelial and mesenchymal tissues (9,10,29-31). Thus, the secretion of FBLN-5 by mesenchymal cells may serve in mediating one set of biological activities in fibroblasts, but a distinct set of activities in neighboring epithelial cells. To test this hypothesis, stable polyclonal populations of mink lung Mv1Lu epithelial cells (16) expressing either GFP or FBLN-5 were generated by retroviral infection and compared for their ability to synthesize DNA by a [³H]thymidine incorporation assay. Similar to 3T3-L1 fibroblasts, expression of FBLN-5 in Mv1Lu epithelial cells failed to affect their sensitivity to TGF-β (FIG. 4A). However, in stark contrast to its stimulation of 3T3-L1 cell proliferation, FBLN-5 expression significantly inhibited Mv1Lu cell DNA synthesis (FIG. 4A). Accordingly, Mv1Lu cell proliferation was also inhibited by addition of recombinant FBLN-5 (data not shown). Thus, these findings demonstrate that FBLN-5 is a negative regulator of epithelial cell growth.

As an additional test for FBLN-5-mediated growth arrest and to establish a mechanism for this effect, a reporter gene assay that measured changes in luciferase expression driven by the cyclin A promoter was performed. Expression of this reporter gene is repressed by TGF-β [Feng, 1995 #1957] and, as expected, TGF-β treatment of Mv1Lu cells repressed their expression of luciferase driven by the cyclin A promoter (FIG. 4B). This response of Mv1Lu cells to TGF-β was not affected by FBLN-5 expression; however, FBLN-5 was able to significantly reduce cyclin A-luciferase expression in unstimulated Mv1Lu cells (FIG. 4B). Taken together, these findings demonstrate that FBLN-5 suppresses cyclin A expression in Mv1Lu cells and thereby reduces DNA synthesis. Moreover, comparing the effects of FBLN-5 expression on DNA synthesis by 3T3-L1 and Mv1Lu cells (i) denotes FBLN-5 as a multifunctional signaling molecule that regulates proliferation in a cell type-specific manner, and (ii) supports the hypothesis that FBLN-5 expression mediates a variety of distinct biological activities in a context-specific manner.

### Example 6

The following example demonstrates that FBLN-5 stimulates Mv1Lu cell AP-1 activity and MAP kinases.

Although FBLN-5 regulates proliferation in a context-specific manner, the inventors suspected that this ability was likely mediated through the stimulation of a signaling pathway whose effect is modified by the particular genetic makeup of an individual cell. Indeed, the inventors'finding that FBLN-5 stimulated MAP kinases in 3T3-L1 cells (see Example 2) prompted the inventors to speculate that FBLN-5 would similarly stimulate MAP kinases in Mv1 Lu cells, leading to enhanced AP-1 activity in them. This hypothesis seemed especially attractive given the fact that (i) MAP kinases phosphorylate and regulate the activity of AP-1 transcription factors (25,26), and (ii) AP-1 activity both enhances and inhibits cell cycle progression (32).

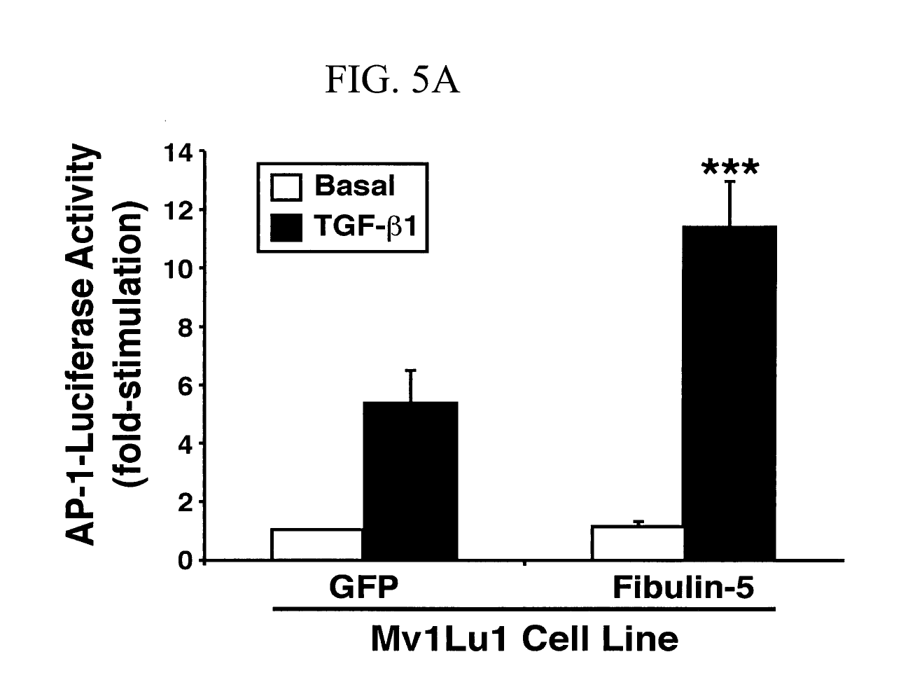

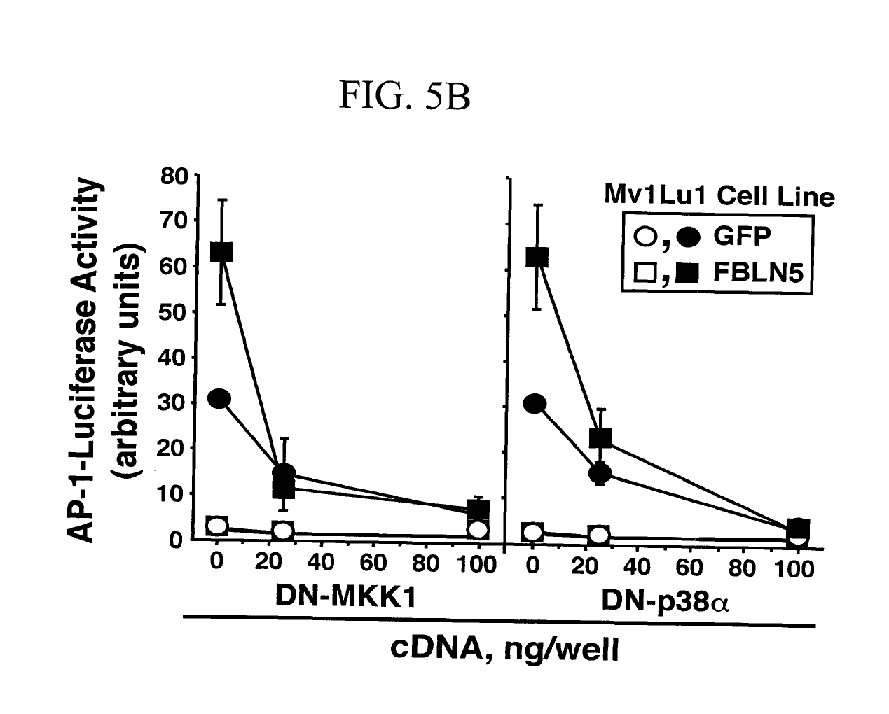

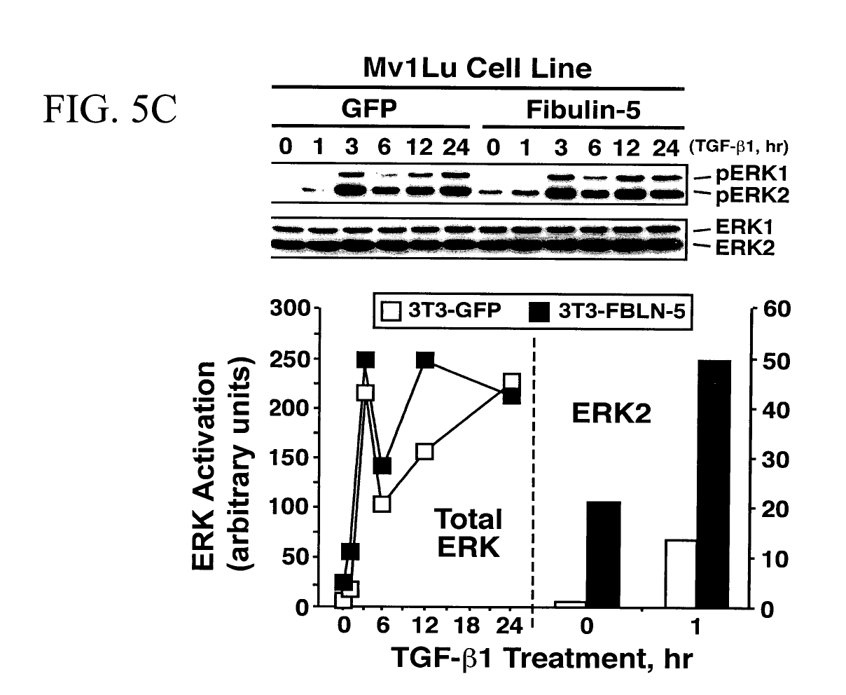

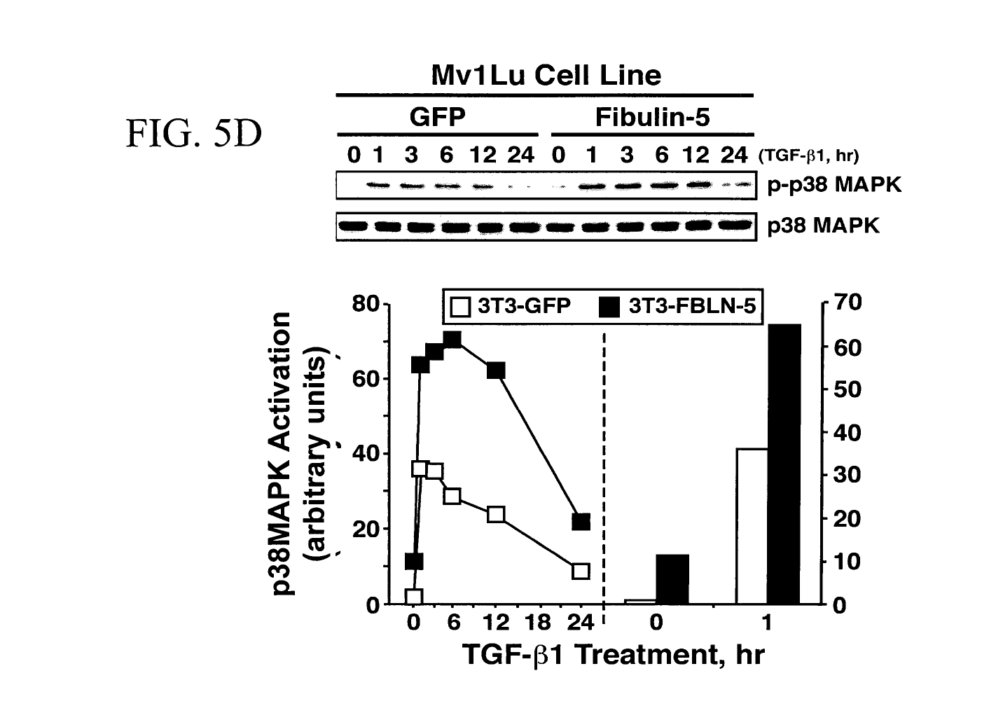

In testing this hypothesis, changes in luciferase expression driven by a synthetic AP-1 promoter [(TGACTAA)₇] were measured. Although FBLN-5 expression failed to affect basal AP-1 activity in Mv1Lu cells, its expression in combination with TGF-β treatment did synergistically stimulate AP-1 activity as compared to control cells stimulated solely with TGF-β (FIG. 5A). The synergistic stimulation of AP-1 activity by FBLN-5 and TGF-β was readily inhibited by overexpression in Mv1Lu cells of dominant-negative versions of either MKK1 or p38 MAPα (FIG. 5B). Thus, this result indicates that ERK1/2 and p38 MAPK activities converge in mediating synergistic stimulation of AP-1 activity in Mv1Lu cells by FBLN-5 and TGF-β. Accordingly, serum-starved FBLN-5-expressing Mv1Lu cells exhibited significantly higher activities of ERK1/2 (FIG. 5C) p38 MAPK (FIG. 5D) as compared to control cells. Lastly, the inventors were unable to detect any changes in JNK activity in serum-starved GFP- or FBLN-5-expressing Mv1Lu cells incubated in the absence or presence of TGF-β (data not shown). Taken together, these findings show that in Mv1Lu cells FBLN-5 and TGF-β collaborate in stimulating AP-1 activation through a ERK1/2- and p38 MAPK-dependent pathway.

### Example 7

The following example shows that Fibulin-5 inhibits angiogenesis and DNA synthesis in endothelial cells, as well as migration and invasion of endothelial cells.

In a two separate experiments, GFP- or Fibulin-5-expressing MB114 cells were cultured on the synthetic basement membrane Matrigel for 3 and 5 days. Photomicrographs showed that Fibulin-5 expression inhibits endothelial cell angiogenic sprouting (data not shown).

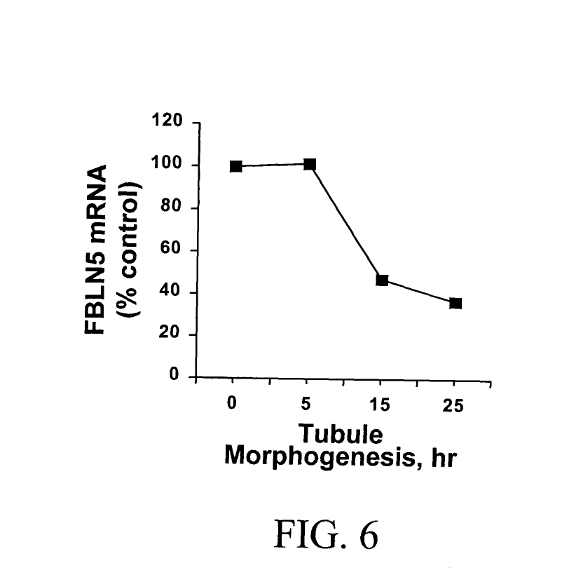

FIG. 6 shows that Fibulin-5 expression is downregulated during MB114 endothelial cell tubule morphogenesis. Briefly, MB 114 endothelial cells were cultured onto Matrigel for varying lengths of time as indicated. Total RNA was isolated, fractionated through agarose/formaldehyde gels, and immobilized to nylon prior to hybridization with radiolabeled human Fibulin-5 cDNA. Results showed that Fibulin-5 mRNA expression was downregulated in tubulating endothelial cells. The differences in RNA loading were monitored by ethidium bromide staining of 28S.

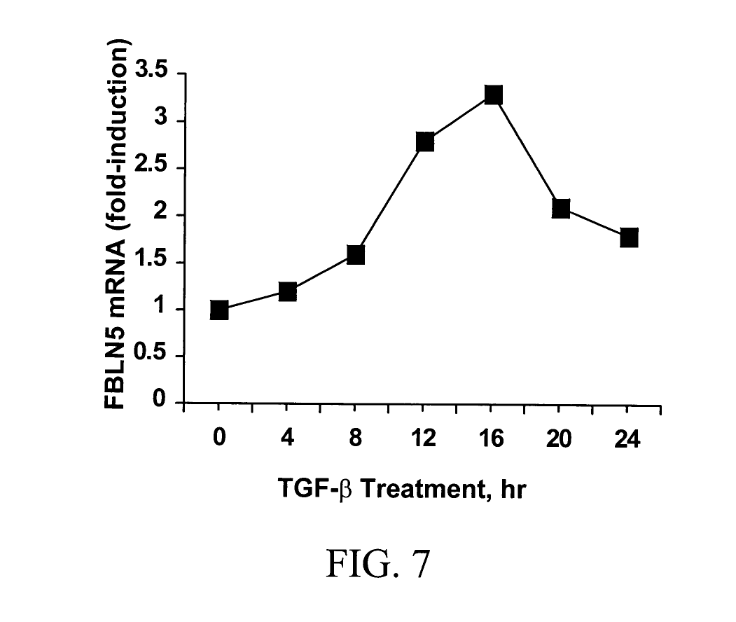

FIG. 7 shows that TGF-β stimulates Fibulin-5 expression in MB114 endothelial cells. Briefly, MB114 endothelial cells were treated with TGF-β1 for varying lengths of time as indicated. Total RNA was isolated, fractionated through agarose/formaldehyde gels, and immobilized to nylon prior to hybridization with radiolabeled human Fibulin-5 cDNA. Results showed that Fibulin-5 mRNA expression is stimulated by TGF-β, with a peak at 16 hours.

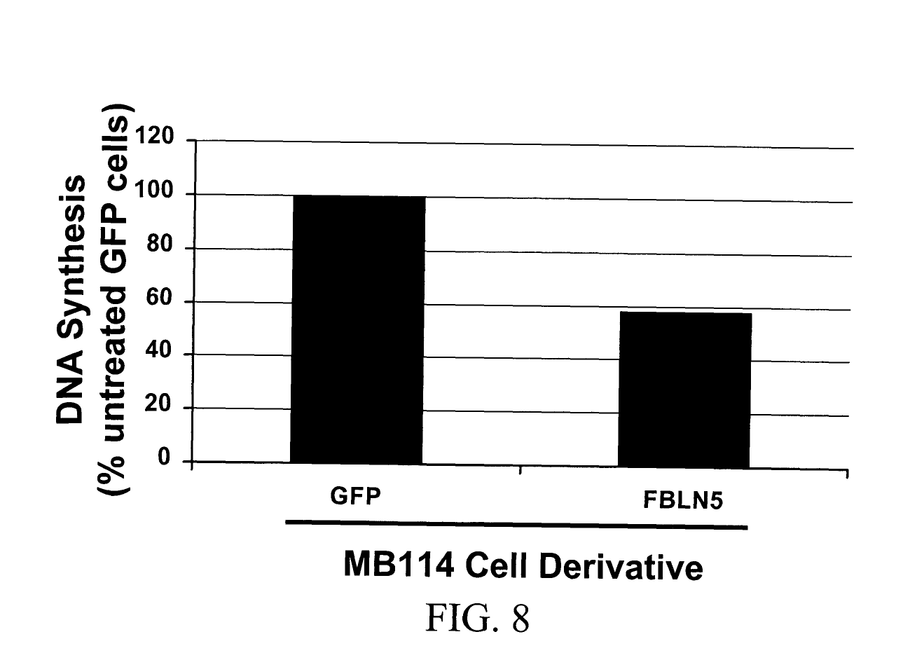

FIG. 8 depicts an experiment showing that Fibulin-5 inhibits endothelial cell DNA synthesis. Briefly, GFP- or Fibulin-5-expressing MB114 endothelial cells were cultured for 24 hr prior to additional of [³H] thymidine to label cellular DNA. FIG. 8 shows reduced DNA synthesis in endothelial cells expressing Fibulin-5.

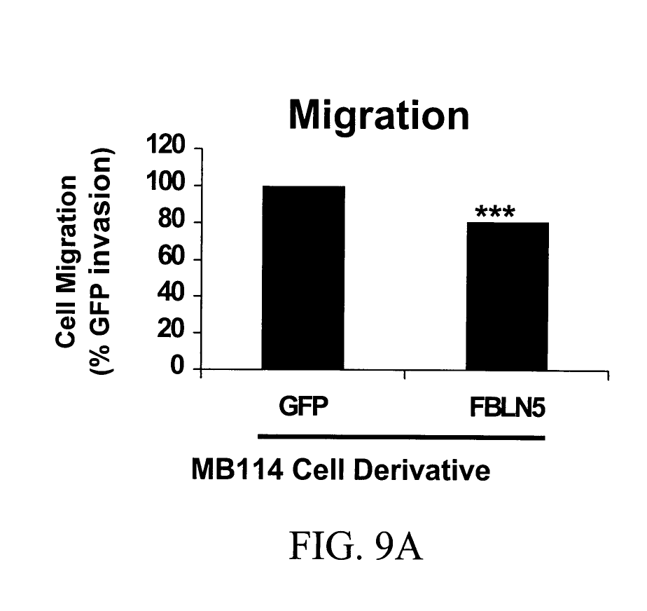

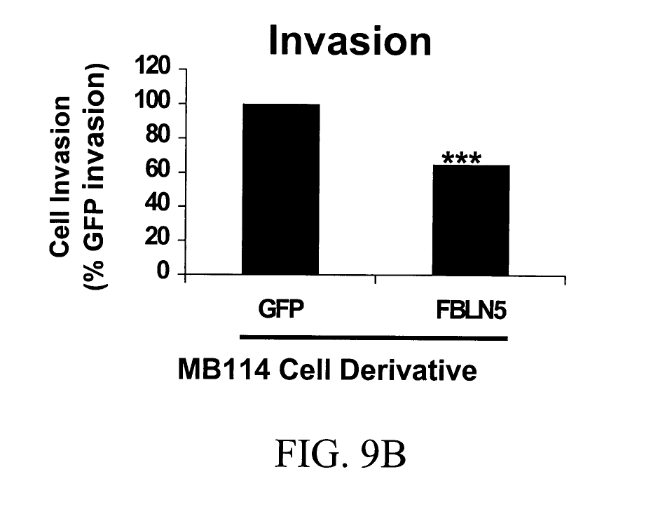

The experimental results depicted in FIGS. 9A and 9B demonstrate that Fibulin-5 inhibits MB114 endothelial cell migration and invasion. In this experiment, GFP- or Fibulin-5-expressing MB114 endothelial cells were allowed to migrate for 24 hr to fibronectin (FIG. 9A), or to invade for 48 hr through synthetic Matrigel basement membranes (FIG. 9B). Migrating or invading MB114 cells were visualized by crystal violet staining and counted by light microscopy. The results show that Fibulin-5 expression significantly inhibits endothelial cell migration and invasion.

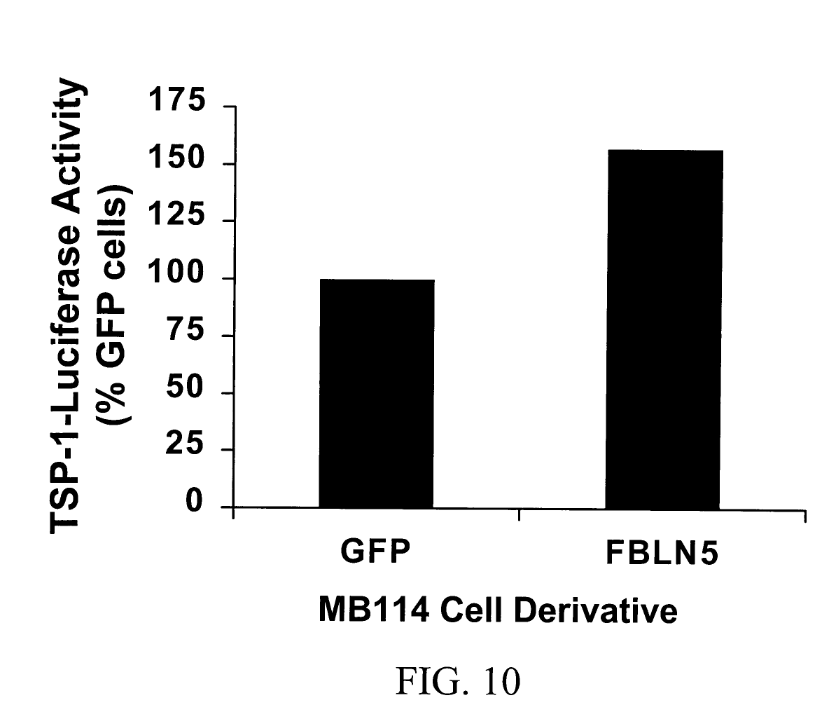

The experimental results depicted in FIG. 10 show that Fibulin-5 stimulates endothelial cells to express the angiostatic protein, thrombospondin-1 (TSP-1). Briefly, GFP- or Fibulin-5 -expressing MB114 cells were transiently transfected with the a luciferase reporter gene whose expression is driven by the thrombospondin-1 promoter, and with pCMV-β-gal to control for differences in transfection efficiency. 48 hours post-transfection, the cells were harvested and processed to measure luciferase and β-gal activities contained in detergent-solubilized cell extracts. FIG. 10 shows that Fibulin-5 expression significantly induces endothelial cell expression of the anti-angiogenic molecule, thrombospondin-1.

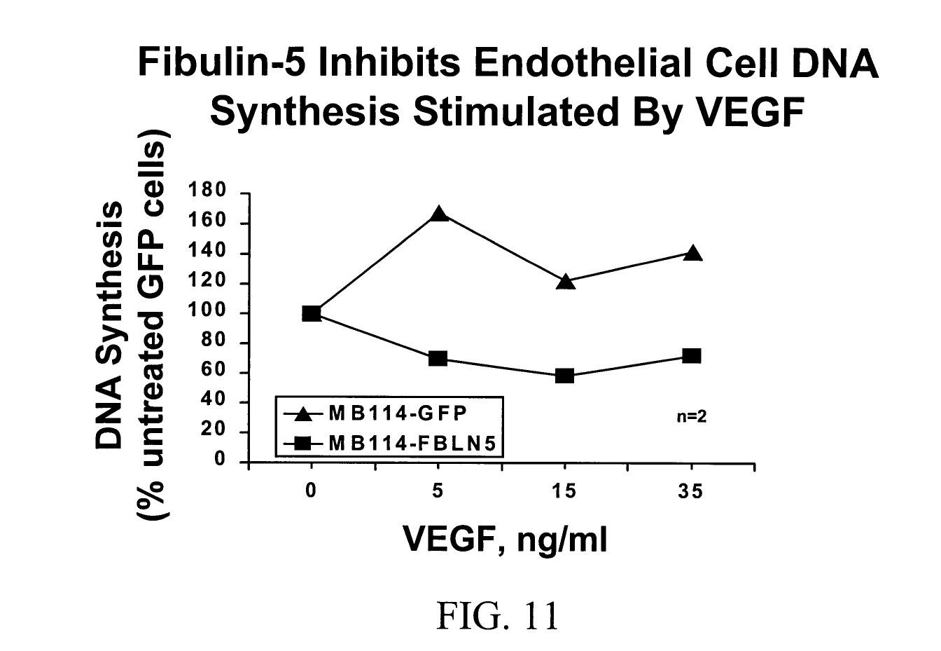

The experimental results shown in FIG. 11 show that Fibulin-5 inhibits endothelial cell DNA synthesis stimulated by VEGF. In this experiment, GFP- or Fibulin-5-expressing MB114 endothelial cells were stimulated for 24 hr with increasing concentrations of VEGF as indicated. Afterward, [³H]thymidine was added to cell cultures to label cellular DNA.

FIG. 11 shows that Fibulin-5 expression inhibits endothelial cell DNA synthesis stimulated by VEGF.

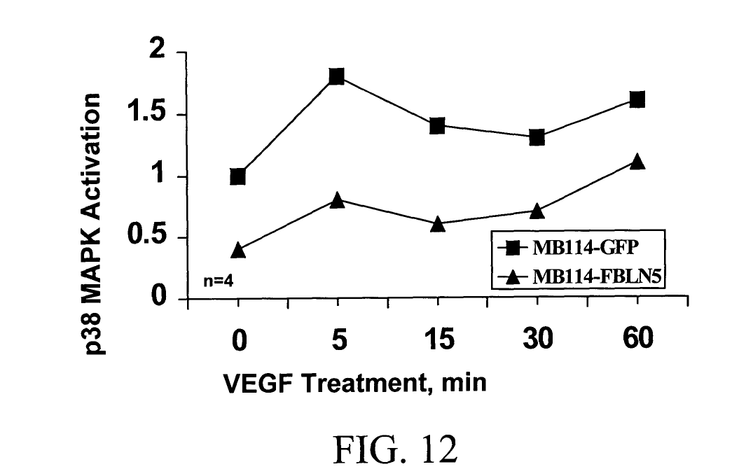

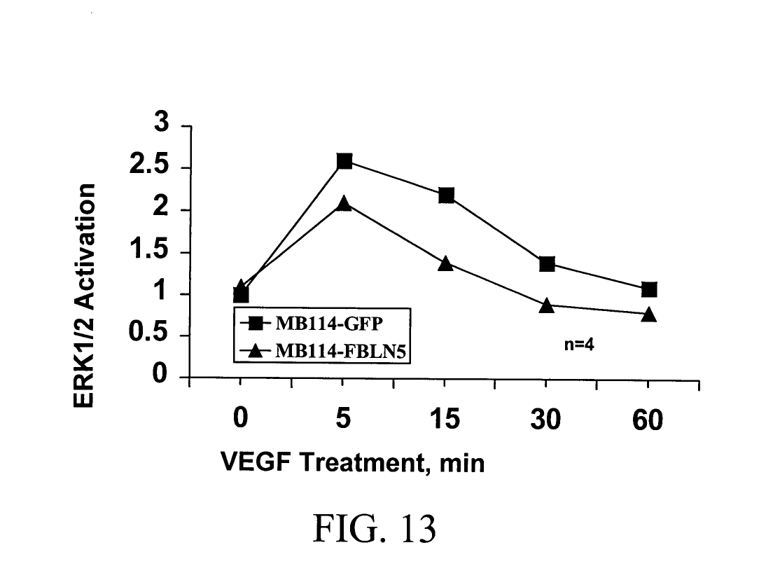

Further experiments demonstrated that Fibulin-5 inhibits p38 MAPK and ERK1/2 activation stimulated by VEGF in MB114 endothelial cells. In these experiments, quiescent GFP- or Fibulin-5-expressing MB114 endothelial cells were stimulated with VEGF for 0-60 min as indicated in FIGS. 12 and 13. Afterward, detergent-solubilized cell extracts were prepared, fractionated through SDS-PAGE, and transferred electrophoretically to nitrocellulose. Immobilized proteins were probed with phospho-specific antibodies against p38 MAPK and ERK1/2. The resulting immunocomplexes were visualized by enhanced chemiluminescence. Differences in protein loading were monitored by reprobing stripped membranes with polyclonal antibodies against p38 MAPK (FIG. 12) and ERK1/2 (FIG. 13). The results show that Fibulin-5 expression inhibits endothelial cell activation of MAP kinases stimulated by VEGF.

Each publication or other reference disclosed below and elsewhere herein is incorporated herein by reference in its entirety.

### References

1. Balbona, K., Tran, H., Godyna, S., Ingham, K. C., Strickland, D. K., and Argraves, W. S. (1992) J. Biol. Chem. 267, 20120-20125

2. Pan, T. C., Sasaki, T., Zhang, R. Z., Fassler, R., Timpl, R., and Chu, M. L. (1993) J. Cell Biol. 123, 1269-1277

3. Kostka, G., Giltay, R., Bloch, W., Addicks, K., Timpl, R., Fassler, R., and Chu, M. L. (2001) Mol. Cell. Biol. 21, 7025-7034

4. Nakamura, T., Lozano, P. R., Ikeda, Y., Iwanaga, Y., Hinek, A., Minamisawa, S., Cheng, C. F., Kobuke, K., Dalton, N., Takada, Y., Tashiro, K., Ross Jr, J., Honjo, T., and Chien, K. R. (2002) Nature 415, 171-175

5. Yanagisawa, H., Davis, E. C., Starcher, B. C., Ouchi, T., Yanagisawa, M., Richardson, J. A., and Olson, E. N. (2002) Nature 415, 168-171

6. Zhang, H. Y., Timpl, R., Sasaki, T., Chu, M. L., and Ekblom, P. (1996) Dev. Dyn. 205, 348-364

7. Tran, H., Tanaka, A., Litvinovich, S. V., Medved, L. V., Haudenschild, C. C., and Argraves, W. S. (1995) J. Biol. Chem. 270, 19458-19464

8. Roark, E. F., Keene, D. R., Haudenschild, C. C., Godyna, S., Little, C. D., and Argraves, W. S. (1995) J. Histochem. Cytochem. 43, 401-411

9. Kowal, R. C., Richardson, J. A., Miano, J. M., and Olson, E. N. (1999) Circ. Res. 84, 1166-1176

10. Nakamura, T., Ruiz-Lozano, P., Lindner, V., Yabe, D., Taniwaki, M., Furukawa, Y., Kobuke, K., Tashiro, K., Lu, Z., Andon, N. L., Schaub, R., Matsumori, A., Sasayama, S., Chien, K. R., and Honjo, T. (1999) J. Biol. Chem. 274, 22476-22483

11. Ghaffari, S., Wu, H., Gerlach, M., Han, Y., Lodish, H. F., and Daley, G. Q. (1999) Proc. Natl. Acad. Sci. USA 96, 13186-13190

12. Pandey, A., Podtelejnikov, A. V., Blagoev, B., Bustelo, X. R., Mann, M., and Lodish, H. F. (2000) Proc. Natl. Acad. Sci. USA 97, 179-184

13. Kalume, D. E., Stenflo, J., Czerwiec, E., Harnbe, B., Furie, B. C., Furie, B., and Roepstorff, P. (2000) J. Mass Spectrom. 35, 145-156

14. Naviaux, R. K., Costanzi, E., Haas, M., and Verma, I. M. (1996) J. Virol. 70, 5701-5705

15. Hua, X., Liu, X., Ansari, D. O., and Lodish, H. F. (1998) Genes Dev. 12, 3084-3095

16. Liu, X., Sun, Y., Constantinescu, S. N., Karam, E., Weinberg, R. A., and Lodish, H. F. (1997) Proc. Natl. Acad. Sci. USA 94, 10669-10674

17. Edlund, M., Miyamoto, T., Sikes, R. A., Ogle, R., Laurie, G. W., Farach-Carson, M. C., Otey, C. A., Zhau, H. E., and Chung, L. W. (2001) Cell Growth Differ. 12, 99-107

18. Qing, J., Maher, V. M., Tran, H., Argraves, W. S., Dunstan, R. W., and McCormick, J. J. (1997) Oncogene 15, 2159-2168

19. Schiemann, W. P., Pfeifer, W. M., Levi, E., Kadin, M. E., and Lodish, H. F. (1999) Blood 94, 2854-2861

20. Blobe, G. C., Schiemann, W. P., and Lodish, H. F. (2000) N. Engl. J. Med. 342, 1350-1358

21. Massague, J. (1998) Annu. Rev. Biochem. 67, 753-791

22. Gallagher, W. M., Argentini, M., Sierra, V., Bracco, L., Debussche, L., and Conseiller, E. (1999) Oncogene 18, 3608-3616

23. Lecka-Czemik, B., Lumpkin, C. K., Jr., and Goldstein, S. (1995) Mol. Cell. Biol. 15, 120-128

24. Hayashido, Y., Lucas, A., Rougeot, C., Godyna, S., Argraves, W. S., and Rochefort, H. (1998) Int. J. Cancer 75, 654-658

25. Garrington, T. P., and Johnson, G. L. (1999) Curr. Opin. Cell Biol. 11,211-218

26. Chang, L., and Karin, M. (2001) Nature 410, 37-40

27. Aplin, A. E., Howe, A. K., and Juliano, R. L. (1999) Curr. Opin. Cell Biol. 11, 737-744

28. Gallagher, W. M., Greene, L. M., Ryan, M. P., Sierra, V., Berger, A., Laurent-Puig, P., and Conseiller, E. (2001) FEBS Lett. 489, 59-66

29. Spence, S. G., Argraves, W. S., Walters, L., Hungerford, J. E., and Little, C. D. (1992) Dev. Biol. 151, 473-484

30. Miosge, N., Gotz, W., Sasaki, T., Chu, M. L., Timpl, R., and Herken, R. (1996) Histochem. J. 28, 109-116

31. Zhang, H. Y., Chu, M. L., Pan, T. C., Sasaki, T., Timpl, R., and Ekblom, P. (1995) Dev. Biol. 167, 18-26

32. Shaulian, E., and Karin, M. (2001) Oncogene 20, 2390-2400

33. Fassler, R., Sasaki, T., Timpl, R., Chu, M. L., and Werner, S. (1996) Exp. Cell Res. 222,111-116

34. Pepper, M. S. (1997) Cytokine Growth Factor Rev. 8, 21-43

35. Bhowmick, N. A., Zent, R., Ghiassi, M., McDonnell, M., and Moses, H. L. (2001) J. Biol. Chem. 276, 46707-46713

36. Massague, J., Blain, S. W., and Lo, R. S. (2000) Cell 103, 295-309

37. Clinton, G. M., Rougeot, C., Derancourt, J., Roger, P., Defrenne, A., Godyna, S., Argraves, W. S., and Rochefort, H. (1996) Proc. Natl. Acad. Sci. USA 93, 316-320

38. Roger, P., Pujol, P., Lucas, A., Baldet, P., and Rochefort, H. (1998) Am. J. Pathol. 153, 1579-1588

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed:

1. A method for assessing the tumorigenicity of cells in a patient, comprising:
a) detecting a level of expression or activity of Fibulin-5 in a test sample from a patient to be diagnosed; and
b) comparing the level of expression or activity of Fibulin-5 in the test sample to a baseline level of Fibulin-5 expression or activity established from a control sample;
wherein detection of a statistically significant difference in Fibulin-5 expression or activity in the test sample, as compared to the baseline level of Fibulin-5 expression or biological activity, is an indicator of a difference in the tumorigenicity or potential therefore of cells in the test sample as compared to cells in the control sample.

2. The method of claim 1, wherein the step of detecting comprises detecting Fibulin-5 mRNA transcription by cells in the test sample.

3. The method of claim 2, wherein the step of detecting is by a method selected from the group consisting of polymerase chain reaction (PCR), reverse transcriptase-PCR (RT-PCR), in situ hybridization, Northern blot, sequence analysis, gene microarray analysis, and detection of a reporter gene.

4. The method of claim 1, wherein the step of detecting comprises detecting Fibulin-5 protein in the test sample.

5. The method of claim 4, wherein the step of detecting is by a method selected from the group consisting of immunoblot, enzyme-linked immunosorbant assay (ELISA), radioimmunoassay (RIA), immunoprecipitation, immunohistochemistry and immunofluorescence.

6. The method of claim 1, wherein the step of detecting comprises detecting Fibulin-5 biological activity in the test sample.

7. The method of claim 6, wherein the step of detecting comprises measuring proliferation of cells expressing Fibulin-5, detecting DNA synthesis in cells expressing Fibulin-5, detecting MAP kinase activity in cells expressing Fibulin-5, detecting MAP kinase activity in the presence of the test sample, and measuring migration and invasion ability of fibroblasts expressing Fibulin-5.

8. The method of claim 1, wherein the test sample is from a source selected from the group consisting of: breast, kidney, ovary, colon, and uterus, in the patient.

9. The method of claim 1, wherein the test sample is a fibroblast cell sample.

10. The method of claim 1, wherein detection of a statistically significant difference in the level of Fibulin-5 expression or activity in the test sample as compared to the baseline level, with a confidence of p<0.05, indicates that the cells in the test sample have a difference in tumorigenicity or potential therefore as compared to the control sample.

11. The method of claim 1, wherein a detection of an at least about 10% difference in the level of Fibulin-5 expression or activity in the test sample as compared to the baseline level, with a confidence of p<0.05, indicates that the cells in the test sample have a difference in tumorigenicity or potential therefore as compared to the control sample.

12. The method of claim 1, wherein a detection of an at least about 30% difference in the level of Fibulin-5 expression or activity in the test sample as compared to the baseline level, with a confidence of p<0.05, indicates that the cells in the test sample have a difference in tumorigenicity or potential therefore as compared to the control sample.

13. The method of claim 1, wherein a detection of an at least about 50% difference in the level of Fibulin-5 expression or activity in the test sample as compared to the baseline level, with a confidence of p<0.05, indicates that the cells in the test sample have a difference in tumorigenicity or potential therefore as compared to the control sample.

14. The method of claim 1, wherein a detection of an at least about 1.5 fold difference in the level of Fibulin-5 expression or activity in the test sample as compared to the baseline level, with a confidence of p<0.05, indicates that the cells in the test sample have a difference in tumorigenicity or potential therefore as compared to the control sample.

15. The method of claim 1, wherein the test sample is from a patient being diagnosed for cancer and wherein the baseline level is established from a control sample that is established as non-tumorigenic.

16. The method of claim 15, wherein, when the Fibulin-5 expression or biological activity detected in step (b) is statistically significantly different as compared to the baseline level, the method further comprises:
c) comparing the Fibulin-5 expression or activity of the test sample as detected in step (b) to levels of Fibulin-5 expression or activity from a panel of tumor-positive control samples, wherein each of the tumor-positive control samples is correlated with a different stage of tumor development; and,
d) identifying a level of Fibulin-5 expression or activity from one of the tumor-positive control samples which is statistically significantly most similar to the level of Fibulin-5 expression or biological activity detected in step, to diagnose a stage of tumor development in the patient.

17. The method of claim 15, wherein the test sample is not a fibroblast cell sample, and wherein a decrease in the level of Fibulin-5 expression or activity of the test sample as compared to the baseline level of expression or activity indicates that cells in the test sample are predicted to be tumorigenic or predisposed to becoming tumorigenic.

18. The method of claim 1, wherein the test sample is from a patient who is known to have cancer, and wherein the baseline level comprises a first level of Fibulin-5 expression or activity from a previous tumor cell sample from the patient and a second level of Fibulin-5 expression or activity established from a cell sample that is non-tumorigenic;
wherein a statistically significant change in the level of Fibulin-5 expression or activity in the test sample toward the baseline level established from the non-tumorigenic cell sample, as compared to the baseline level of expression or activity from the previous tumor cell sample, indicates that the test sample is less tumorigenic than the previous tumor cell sample; and
wherein a statistically significant change in the level of Fibulin-5 expression or activity in the test sample away from the level established from the non-tumorigenic cell sample, as compared to the baseline level of expression or activity, indicates that the test sample is more tumorigenic than the previous tumor cell sample.

19. The method of claim 18, wherein the method further comprises a step (c) of modifying cancer treatment for the patient based on whether an increase or decrease in tumorigenicity is indicated in step (b).

20. The method of claim 1, wherein the baseline level is established by a method selected from the group consisting of:
(1) establishing a baseline level of Fibulin-5 expression or activity in an autologous control sample from the patient, wherein the autologous sample is from a same cell type, tissue type or bodily fluid type as the test sample of step (a);
(2) establishing a baseline level of Fibulin-5 expression or activity from at least one previous detection of Fibulin-5 expression or activity in a previous test sample from the patient, wherein the previous test sample was of a same cell type, tissue type or bodily fluid type as the test sample of step (a); and,
(3) establishing a baseline level of Fibulin-5 expression or activity from an average of control samples of a same cell type, tissue type or bodily fluid type as the test sample of step (a), the control samples having been obtained from a population of matched individuals.

21. An assay kit for assessing the tumorigenicity of cells in a patient, comprising:
a) a means for detecting Fibulin-5 expression or activity in a test sample; and
b) a means for detecting a control marker characteristic of a cell or tissue type that is in the test sample or that is secreted into the test sample by the cell or tissue.

22. The assay kit of claim 21, wherein the means of (a) is selected from the group consisting of: a hybridization probe of at least about 8 nucleotides that hybridizes under stringent hybridization conditions to a nucleic acid molecule encoding Fibulin-5 or a fragment thereof; an oligonucleotide primer for amplification of mRNA encoding Fibulin-5 or a fragment thereof; and an antibody that selectively binds to Fibulin-5.

23. The assay kit of claim 21, wherein the means of (b) is selected from the group consisting of: a hybridization probe of at least about 8 nucleotides that hybridizes under stringent hybridization conditions to a nucleic acid molecule encoding the control marker or a fragment thereof; an oligonucleotide primer for amplification of mRNA encoding the control marker or a fragment thereof; and an antibody that selectively binds to the control marker.

24. The assay kit of claim 21, wherein the means of (a) and (b) are suitable for use in a method of detection selected from the group consisting of immunohistochemistry and immunofluorescence.

25. A method to identify a compound useful for inhibition of tumor growth or malignancy, comprising:
a) detecting an initial level of Fibulin-5 expression or activity in a tumor cell or soluble product derived therefrom;
b) contacting the tumor cell with a test compound;
c) detecting a level of Fibulin-5 expression or activity in the tumor cell or soluble product derived therefrom after contact of the tumor cell with the compound; and,
d) selecting a compound that changes the level of Fibulin-5 expression or activity in the tumor cell or soluble product therefrom, as compared to the initial level of Fibulin-5 expression or activity, toward a baseline level of Fibulin-5 expression or activity established from a non-tumor cell, wherein the selected compound is predicted to be useful for inhibition of tumor growth or malignancy.

26. A method to reduce angiogenesis in a tissue of a patient, comprising increasing the expression or biological activity of Fibulin-5 in the cells of the tissue.

27. The method of claim 26, comprising administering Fibulin-5 or a biologically active homologue or analog thereof to the patient.

28. The method of claim 26, comprising expressing a recombinant nucleic acid molecule encoding Fibulin-5 or a homologue thereof in the tissue of the patient.

29. A method to reduce tumorigenicity in a patient, comprising increasing the expression or biological activity of Fibulin-5 in targeted tumor cells of the patient.

30. The method of claim 29, wherein the tumor cells are from a tissue selected from the group consisting of: breast, ovary, kidney, colon, and uterus.

31. The method of claim 29, comprising administering Fibulin-5 or a biologically active homologue or analog thereof to the patient.

32. The method of claim 29, comprising expressing a recombinant nucleic acid molecule encoding Fibulin-5 or a homologue thereof in the tissue of the patient.

33. A method to reduce tumorigenicity of a fibrosarcoma in a patient, comprising decreasing the expression or biological activity of Fibulin-5 in fibrosarcoma cells of the patient.

34. A method to identify a regulator of transforming growth factor β (TGFβ), comprising:
a) contacting a cell that expresses TGFβ and Fibulin-5 with a putative regulatory compound;
b) detecting the expression of Fibulin-5 in the cell; and
c) comparing the expression of Fibulin-5 after contact with the compound to the expression of Fibulin-5 before contact with the compound, wherein detection of a change in the expression of Fibulin-5 in the cells after contact with the compound as compared to before contact with the compound indicates that the compound is a putative regulator of TGFβ.


SEQUENCE LISTING (5 sequences)

SEQ ID NO: 1 - protein, 11 aa, Mus musculus
YPGAYYIFQIK

SEQ ID NO: 2 - DNA, 1347 bp, Homo sapiens
  CDS (1)..(1347)
atgccaggaataaaaaggatactcactgttaccattctggctctctgtcttccaagccctgggaatgcacaggcacagtgcacgaatggctttgacctggatcgccagtcaggacagtgtttagatattgatgaatgccgaaccatccccgaggcctgccgaggagacatgatgtgtgttaaccaaaatggcgggtatttatgcattccccggacaaaccctgtgtatcgagggccctactcgaacccctactcgaccccctactcaggtccgtacccagcagctgccccaccactctcagctccaaactatcccacgatctccaggcctcttatatgccgctttggataccagatggatgaaagcaaccaatgtgtggatgtggacgagtgtgcaacagattcccaccagtgcaaccccacccagatctgcatcaatactgaaggcgggtacacctgctcctgcaccgacggatattggcttctggaaggccagtgcttagacattgatgaatgtcgctatggttactgccagcagctctgtgcgaatgttcctggatcctattcttgtacatgcaaccctggttttaccctcaatgaggatggaaggtcttgccaagatgtgaacgagtgtgccaccgagaacccctgcgtgcaaacctgcgtcaacacctacggctctttcatctgccgctgtgacccaggatatgaacttgaggaagatggcgttcattgcagtgatatggacgagtgcagcttctctgagttcctctgccaacatgagtgtgtgaaccagcccggcacatacttctgctcctgccctccaggctacatcctgctggatgacaaccgaagctgccaagacatcaacgaatgtgagcacaggaaccacacgtgcaacctgcagcagacgtgctacaatttacaagggggcttcaaatgcatcgaccccatccgctgtgaggagccttatctgaggatcagtgataaccgctgtatgtgtcctgctgagaaccctggctgcagagaccagccctttaccatcttgtaccgggacatggacgtggtgtcaggacgctccgttcccgctgacatcttccaaatgcaagccacgacccgctaccctggggcctattacattttccagatcaaatctgggaatgagggcagagaattttacatgcggcaaacgggccccatcagtgccaccctggtgatgacacgccccatcaaagggccccgggaaatccagctggacttggaaatgatcactgtcaacactgtcatcaacttcagaggcagctccgtgatccgactgcggatatatgtgtcgcagtacccattctga

SEQ ID NO: 3 - protein, 448 aa, Homo sapiens
MPGIKRILTVTILALCLPSPGNAQAQCTNGFDLDRQSGQCLDIDECRTIPEACRGDMMCVNQNGGYLCIPRTNPVYRGPYSNPYSTPYSGPYPAAAPPLSAPNYPTISRPLICRFGYQMDESNQCVDVDECATDSHQCNPTQICINTEGGYTCSCTDGYWLLEGQCLDIDECRYGYCQQLCANVPGSYSCTCNPGFTLNEDGRSCQDVNECATENPCVQTCVNTYGSFICRCDPGYELEEDGVHCSDMDECSFSEFLCQHECVNQPGTYFCSCPPGYILLDDNRSCQDINECEHRNHTCNLQQTCYNLQGGFKCIDPIRCEEPYLRISDNRCMCPAENPGCRDQPFTILYRDMDVVSGRSVPADIFQMQATTRYPGAYYIFQIKSGNEGREFYMRQTGPISATLVMTRPIKGPREIQLDLEMITVNTVINFRGSSVIRLRIYVSQYPF

SEQ ID NO: 4 - DNA, 1347 bp, Homo sapiens
  CDS (1)..(1347)
atgccaggattaaaaaggatactcactgttaccatcttggcactctggcttccacatcctgggaatgcacagcagcagtgcacaaacggctttgacctggaccgccagtcaggacagtgtctagatattgatgaatgccggaccatccctgaggcttgtcgtggggacatgatgtgtgtcaaccagaatggcgggtatttgtgcatccctcgaaccaacccagtgtatcgagggccttactcaaatccctactctacatcctactcaggcccatacccagcagcggccccaccagtaccagcttccaactaccccacgatttcaaggcctcttgtctgccgctttgggtatcagatggatgaaggcaaccagtgtgtggatgtggacgagtgtgcaacagactcacaccagtgcaaccctacccagatctgtatcaacactgaaggaggttacacctgctcctgcaccgatgggtactggcttctggaagggcagtgcctagatattgatgaatgtcgctatggttactgccagcagctctgtgcaaatgttccaggatcctattcctgtacatgcaaccctggtttcaccctcaacgacgatggaaggtcttgccaagatgtgaacgagtgcgaaactgagaatccctgtgttcagacctgtgtcaacacctatggctctttcatctgccgctgtgacccaggatatgaacttgaggaagatggcattcactgcagtgatatggacgagtgcagcttctccgagttcctctgtcaacacgagtgtgtgaaccagccgggctcatacttctgctcgtgccctccaggctacgtcctgttggatgataaccgaagctgccaggatatcaatgaatgtgagcaccgaaaccacacgtgtacctcactgcagacttgctacaatctacaagggggcttcaaatgtattgatcccatcagctgtgaggagccttatctgctgattggtgaaaaccgctgtatgtgtcctgctgagcacaccagctgcagagaccagccattcaccatcctgtatcgggacatggatgtggtgtcaggacgctccgttcctgctgacatcttccagatgcaagcaacaacccgataccctggtgcctattacattttccagatcaaatctggcaacgagggtcgagagttctatatgcggcaaacagggcctatcagtgccaccctggtgatgacacgccccatcaaagggcctcgggacatccagctggacttggagatgatcactgtcaacactgtcatcaacttcagaggcagctccgtgatccgactgcggatatatgtgtcgcagtatccgttctga

SEQ ID NO: 5 - protein, 448 aa, Homo sapiens
MPGLKRILTVTILALWLPHPGNAQQQCTNGFDLDRQSGQCLDIDECRTIPEACRGDMMCVNQNGGYLCIPRTNPVYRGPYSNPYSTSYSGPYPAAAPPVPASNYPTISRPLVCRFGYQMDEGNQCVDVDECATDSHQCNPTQICINTEGGYTCSCTDGYWLLEGQCLDIDECRYGYCQQLCANVPGSYSCTCNPGFTLNDDGRSCQDVNECETENPCVQTCVNTYGSFICRCDPGYELEEDGIHCSDMDECSFSEFLCQHECVNQPGSYFCSCPPGYVLLDDNRSCQDINECEHRNHTCTSLQTCYNLQGGFKCIDPISCEEPYLLIGENRCMCPAEHTSCRDQPFTILYRDMDVVSGRSVPADIFQMQATTRYPGAYYIFQIKSGNEGREFYMRQTGPISATLVMTRPIKGPRDIQLDLEMITVNTVINFRGSSVIRLRIYVSQYPF